(19) United States
(12) Patent Application Publication
(10) Pub. No.: US 2022/0186229 A1
(43) Pub. Date: Jun. 16, 2022

(54) METHODS AND COMPOSITIONS FOR INHIBITING EXPRESSION OF CYP27A1

(71) Applicant: Dicema Pharmaceuticals, Inc., Lexington, MA (US)

(72) Inventor: Utsav SAXENA, Watertown, MA (US)

(21) Appl. No.: 17/310,579

(22) PCT Filed: Feb. 7, 2020
(86) PCT No.: PCT/US2020/017129

§ 371 Date: Aug. 12, 2021

Related U.S. Application Data
(60) Provisional application No. 62/804,410, filed on Feb. 12, 2019.

(51) Int. Cl. C12N 15/113; A61K 31/7105; A61P 1/16; C12N 15/11; C12N 9/02

(52) U.S. Cl. CPC C12N 15/1137; A61K 31/7105; A61P 1/16; C12N 15/111; C12N 9/0077; C12N 2310/531; C12N 2310/14; C12N 2310/315; C12N 2310/321; C12N 2310/3515; C12N 2320/11; C12Y 114/15

(57) ABSTRACT

This disclosure relates to oligonucleotides, compositions and methods useful for reducing CYP27A1 expression, particularly in hepatocytes. Disclosed oligonucleotides for the reduction of CYP27A1 expression may be double-stranded or single-stranded and may be modified for improved characteristics such as stronger resistance to nucleases and lower immunogenicity. Disclosed oligonucleotides for the reduction of CYP27A1 expression may also include targeting ligands to target a particular cell or organ, such as the hepatocytes of the liver, and may be used to treat hepatobiliary disease and related conditions (e.g., liver fibrosis).

### RELATED APPLICATIONS

This application claims the benefit under 35 U.S.C. § 119(e) of U.S. Provisional Application Ser. No. 62/804,410, filed Feb. 12, 2019, the entire contents of which are incorporated herein by reference.

### FIELD OF THE INVENTION

The present application relates to oligonucleotides and uses thereof, particularly uses relating to the modulation of metabolic functions of the liver.

### REFERENCE TO THE SEQUENCE LISTING

The present application is being filed along with a Sequence Listing in electronic format. The Sequence Listing is provided as a file entitled 400930-012WO_SEQ.txt created on Feb. 6, 2020 which is 162 kilobytes in size. The information in electronic format of the sequence listing is incorporated herein by reference in its entirety.

### BACKGROUND OF THE INVENTION

Among the many metabolic functions performed by the liver, the synthesis and flow of bile are important for the optimal functioning of the enterohepatic systems. Bile is a fluid produced by the liver, stored in the gall bladder and secreted into the intestines, where it helps in the absorption of dietary fat and fat soluble vitamins as well as the excretion of waste products such as bilirubin and excess cholesterol. Bile acids also play roles as hormonal regulators.

The bile acids synthesized in the liver are known as primary bile acids, which are conjugated with glycine or taurine and secreted into the gut. In the colon, the intestinal bacteria, further modifies the bile acids to form secondary bile acids. These secondary bile acids are then absorbed and returned to the liver through enterohepatic circulation. The major primary bile acids are cholic acid and chenodeoxycholic acid, while the major secondary bile acids include deoxycholic acid and lithocholic acid. In addition to these bile acids, muricholic acids may also be present.

The amphipathic nature of bile acids allows them to function as surfactants or detergents; this in turn gives them the ability to form micelles with dietary fats, emulsifying the fats and enhancing their uptake through the intestines. Furthermore, the detergent nature of bile acids contributes to their toxicity.

Total Parenteral Nutrition (“TPN”) is intravenous administration of nutrition, which may include protein, carbohydrate, fat, minerals and electrolytes, vitamins and other trace elements for patients who cannot eat or absorb enough food through tube feeding formula or by mouth to maintain good nutrition status. This is achieved by bypassing the gut. Getting the right nutritional intake in a timely manner can help combat complications and be an important part of a patient's recovery. However, while TPN provides life-saving nutritional support in situations where caloric supply via the enteral route cannot cover the necessary needs of the organism, it does have serious adverse effects, including parenteral nutrition-associated liver disease (PNALD). The development of liver injury associated with PN is multifactorial, including non-specific intestine inflammation, compromised intestinal permeability, and barrier function associated with increased bacterial translocation, primary and secondary cholangitis, cholelithiasis, short bowel syndrome, disturbance of hepatobiliary circulation, lack of enteral nutrition, shortage of some nutrients (proteins, essential fatty acids, choline, glycine, taurine, carnitine, etc.), and toxicity of components within the nutrition mixture itself (glucose, phytosterols, manganese, aluminum, etc.). It has been noted in rodent models that during regular feeding, bile acids activate farnesoid X receptor (FXR) in the gut and enhance the expression of fibroblast growth factor 19 (FGF19) level. (Kumar J. et al., (2014), Newly Identified Mechanisms of Total Parenteral Nutrition Related Liver Injury, ADVANCES IN HEPATOLOGY 1-7).

It is also known that FGF19 regulates bile acid, lipid, and glucose metabolism. Thus, modulators of the FXR-FGF19 pathway could overcome the negative effects on the liver of TPN. Likewise, FXR-regulated enzymes, including cytochrome P450 (CYP) 7A1, CYP8B1 and CYP27A1, CYP3A4, CYP3A11, sulphotransferase 2A1 (SULT2A1) and UDP-glucuronosyltransferase 2B4 (UGT2B4/UGT2B11) participate in the synthesis and metabolism of bile acids. Shifts in the amount of bile acids that lead to their increase has the potential to induce and to potentiate hepatotoxicity through pro-inflammatory mechanisms, membrane damage and cytotoxic reactions and may have consequences for lipid homeostasis. Reduction of bile acid expression by targeting genes such as CYP27A1 through RNAi gene silencing may have the effect of modifying and alleviating such damage and resultant pathologies including PNALD or other affects associated with TPN.

### BRIEF SUMMARY OF THE INVENTION

Aspects of the disclosure relate to compositions and related methods for reducing expression of genes affecting liver metabolic functions, particularly genes affecting bile acid levels in a subject. In some embodiments, the disclosure relates to a recognition that CYP27A1 is a useful target for the treatment of hepatobiliary diseases, particularly such diseases that are associated with bile acid accumulation. In further aspects it has been discovered that oligonucleotides for reducing expression or activity of CYP27A1 are useful for treating conditions in which the accumulation of bile acids in the liver contributes to cellular toxicity (e.g., to toxicity hepatocytes and/or cholangiocytes) and/or promotes liver fibrosis. Accordingly, in some embodiments, the disclosure relates to the use of oligonucleotides, including RNAi oligonucleotides, antisense oligonucleotides, and other similar modalities, for reducing expression or activity of CYP27A1 for the treating of hepatobiliary diseases, including, for example, cholestasis, cholangitis, nonalcoholic steatohepatitis (NASH) and/or alagille syndrome.

In further embodiments, potent RNAi oligonucleotides have been developed for selectively inhibiting CYP27A1 expression in a subject. In some embodiments, the RNAi oligonucleotides are useful for reducing CYP27A1 activity, and thereby decreasing or preventing the accumulation of bile acid in a subject. In some embodiments, key regions of CYP27A1 activity mRNA (referred to as hotspots) have been identified herein that are particularly amenable to targeting using such oligonucleotide-based approaches (See Example 1). In some embodiments, oligonucleotides developed herein to inhibit CYP27A1 expression are useful for reducing or preventing liver fibrosis associated with bile acid accumulation (see, e.g., Example 1, FIG. 7 and FIG. 8).

One aspect of the present disclosure provides oligonucleotides for reducing expression of CYP27A1. In some embodiments, the oligonucleotides comprise an antisense strand comprising a sequence as set forth in any one of SEQ ID NOs: 579-580, 598-614, 763-766, 786, and 788. In some embodiments, the oligonucleotides further comprise a sense strand that comprises a sequence as set forth in any one of SEQ ID NOs: 577-578, 581-597, 759-762, 785, and 787. In some embodiments, the antisense strand consists of a sequence as set forth in any one of SEQ ID NOs: 579-580, 598-614, 763-766, 786, and 788. In some embodiments, the sense strand consists of a sequence as set forth in any one of SEQ ID NOs: 577-578, 581-597, 759-762, 785, and 787.

One aspect of the present disclosure provides oligonucleotides for reducing expression of CYP27A1, in which the oligonucleotides comprise an antisense strand of 15 to 30 nucleotides in length. In some embodiments, the antisense strand has a region of complementarity to a target sequence of CYP27A1 as set forth in any one of SEQ ID NOs: 767-781. In some embodiments, the region of complementarity is at least 15, at least 16, at least 17, at least 18, at least 19, at least 20, at least 21, or at least 22 contiguous nucleotides in length. In some embodiments, the region of complementarity is fully complementary to the target sequence of CYP27A1. In some embodiments, the region of complementarity to CYP27A1 is at least 19 contiguous nucleotides in length. In some embodiments, the sense strand comprises a sequence as set forth in any one of SEQ ID NOs: 577-578, 581-597, 759-762, 785, and 787. In some embodiments, the sense strand consists of a sequence as set forth in any one of SEQ ID NOs: 577-578, 581-597, 759-762, 785, and 787. In some embodiments, the antisense strand comprises a sequence as set forth in any one of SEQ ID NOs: 579-580, 598-614, 763-766, 786, and 788. In some embodiments, the antisense strand consists of a sequence as set forth in any one of SEQ ID NOs: 579-580, 598-614, 763-766, 786, and 788.

In some embodiments, the antisense strand is 19 to 27 nucleotides in length. In some embodiments, the antisense strand is 21 to 27 nucleotides in length. In some embodiments, the oligonucleotide further comprises a sense strand of 15 to 40 nucleotides in length, in which the sense strand forms a duplex region with the antisense strand. In some embodiments, the sense strand is 19 to 40 nucleotides in length. In some embodiments, the antisense strand is 27 nucleotides in length and the sense strand is 25 nucleotides in length. In some embodiments, the duplex region is at least 15, at least 16, at least 17, at least 18, at least 19, at least 20, or at least 21 nucleotides in length. In some embodiments, the antisense strand and sense strand form a duplex region of 25 nucleotides in length.

In some embodiments, an oligonucleotide comprises an antisense strand and a sense strand that are each in a range of 21 to 23 nucleotides in length. In some embodiments, an oligonucleotide comprises a duplex structure in a range of 19 to 21 nucleotides in length. In some embodiments, an oligonucleotide further comprises a 3′-overhang sequence on the antisense strand of two nucleotides in length. In some embodiments, an oligonucleotide comprises a 3′-overhang sequence of one or more nucleotides in length, in which the 3′-overhang sequence is present on the antisense strand, the sense strand, or the antisense strand and sense strand. In some embodiments, an oligonucleotide comprises a 3′-overhang sequence of two nucleotides in length, in which the 3′-overhang sequence is present on the antisense strand, and in which the sense strand is 21 nucleotides in length and the antisense strand is 23 nucleotides in length, such that the sense strand and antisense strand form a duplex of 21 nucleotides in length.

In some embodiments, the sense strand comprises at its 3′-end a stem-loop set forth as: S₁-L-S₂, in which S₁ is complementary to S₂, and in which L forms a loop between S₁ and S₂ of 3 to 5 nucleotides in length.

Another aspect of the present disclosure provides an oligonucleotide for reducing expression of CYP27A1, the oligonucleotide comprising an antisense strand and a sense strand, in which the antisense strand is 21 to 27 nucleotides in length and has a region of complementarity to CYP27A1, in which the sense strand comprises at its 3′-end a stem-loop set forth as: S1-L-S2, in which S1 is complementary to S2, and in which L forms a loop between S1 and S2 of 3 to 5 nucleotides in length, and in which the antisense strand and the sense strand form a duplex structure of at least 19 nucleotides in length but are not covalently linked. In some embodiments, the region of complementarity to CYP27A1 mRNA is fully complementary to at least 15, at least 16, at least 17, at least 18, at least 19, at least 20, or at least 21 contiguous nucleotides of CYP27A1 mRNA. In some embodiments, L is a tetraloop. In some embodiments, L is 4 nucleotides in length. In some embodiments, L comprises a sequence set forth as GAAA.

In some embodiments, an oligonucleotide comprises at least one modified nucleotide. In some embodiments, the modified nucleotide comprises a 2′-modification. In some embodiments, the 2′-modification is a modification selected from: 2′-aminoethyl, 2′-fluoro, 2′-O-methyl, 2′-O-methoxyethyl, and 2′-deoxy-2′-fluoro-β-d-arabinonucleic acid. In some embodiments, all of the nucleotides of an oligonucleotide are modified.

In some embodiments, an oligonucleotide comprises at least one modified internucleotide linkage. In some embodiments, the at least one modified internucleotide linkage is a phosphorothioate linkage. In some embodiments, the 4′-carbon of the sugar of the 5′-nucleotide of the antisense strand comprises a phosphate analog. In some embodiments, the phosphate analog is oxymethyl phosphonate, vinylphosphonate, or malonyl phosphonate.

In some embodiments, at least one nucleotide of an oligonucleotide is conjugated to one or more targeting ligands. In some embodiments, each targeting ligand comprises a carbohydrate, amino sugar, cholesterol, polypeptide, or lipid. In some embodiments, each targeting ligand comprises a N-acetylgalactosamine (GalNAc) moiety. In some embodiments, the GalNac moiety is a monovalent GalNAc moiety, a bivalent GalNAc moiety, a trivalent GalNAc moiety, or a tetravalent GalNAc moiety. In some embodiments, up to 4 nucleotides of L of a stem-loop are each conjugated to a monovalent GalNAc moiety. In other embodiments, a bi-valent, tri-valent or tetravalent GalNac moiety is conjugated to a single nucleotide, e.g., of the nucleotides of L of a stem loop. In some embodiments, the targeting ligand comprises an aptamer.

Another aspect of the present disclosure provides a composition comprising an oligonucleotide of the present disclosure and an excipient. Another aspect of the present disclosure provides a method comprising administering a composition of the present disclosure to a subject. In some embodiments, such methods are useful for attenuating bile acid accumulation in liver of a subject. In some embodiments, such methods are useful for decreasing the extent of liver fibrosis in a subject in need thereof. In some embodiments, such methods are useful for decreasing circulating bile acid concentrations in a subject in need thereof. In some embodiments, such methods are useful for treating hepatobiliary disease. In some embodiments, the subject suffers from PNALD.

Another aspect of the present disclosure provides an oligonucleotide for reducing expression of CYP27A1, the oligonucleotide comprising a sense strand of 15 to 40 nucleotides in length and an antisense strand of 15 to 30 nucleotides in length, in which the sense strand forms a duplex region with the antisense strand, in which the sense strand comprises a sequence as set forth in any one of SEQ ID NOs: 577-578, 581-597, 759-762, 785, and 787 and the antisense strand comprises a complementary sequence selected from SEQ ID NOs: 579-580, 598-614, 763-766, 786, and 788.

In some embodiments, the oligonucleotide comprises a pair of sense and antisense strands selected from a row of the table set forth in Appendix A.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 is a flowchart depicting the experimental design used to select compounds for testing in cell and animal models and to develop oligonucleotides for reducing expression of CYP27A1. SAR: Structure-Activity Relationship.

FIG. 2 is a schematic showing a non-limiting example of a double-stranded oligonucleotide with a nicked tetraloop structure that has been conjugated to four GalNAc moieties (yellow diamonds).

FIG. 3 is a graph showing the percent of CYP27A1 mRNA remaining after a primary oligonucleotide screen conducted using human HepG2 cells used to identify active 25/27mers. Data are normalized to using M15-modified controls using Hs HPRT 517-591(FAM) and Hs SFRS9 594-690 (Hex) assays.

FIGS. 4A and 4B is a set of graphs depicting results of an evaluation of nicked tetraloop oligonucleotides (36/22mers) in human HepG2 cells. Data are normalized to mock-transfected cells using a Hs SFRS9 594-690 (Hex) assay. For both FIGS. 4A and 4B, the “S,” “AS” and “M” designate a sense strand, antisense strand and a modification pattern, respectively; the numbers following the “S” and “AS” represent the SEQ ID NOs; the number following the “M” represents a modification pattern. FIG. 4A shows data for oligonucleotides formed of sense sequences SEQ ID NOs: 577 and 578, and antisense sequences SEQ ID NOs: 579 and 580, respectively. FIG. 4B shows data for oligonucleotides formed of sense sequences SEQ ID NOs: 577 and 581-597, and antisense sequences SEQ ID NOs: 579 and 598-614, respectively. “*” represents oligonucleotides in which the base of the first nucleotide in the 5′ end of the antisense strand is substituted with a uracil.

FIG. 5 is a graph depicting results of an assay evaluating reduction of mouse CYP27A1 expression using nicked tetraloop oligonucleotides and conjugated to GalNAc moieties. The “G” in the names of the oligonucleotides designate that they are conjugated to GalNAc moieties. Data is shown for oligonucleotides formed of sense sequences SEQ ID NOs: 759 to 762, and antisense sequences SEQ ID NOs: 763 to 766, respectively, and having different modification patterns.

FIG. 6 is a graph depicting results of an assay evaluating reduction of human CYP27A1 expression using nicked tetraloop oligonucleotides conjugated to GalNAc moieties. The “G” in the names of the oligonucleotides designate that they are conjugated to GalNAc moieties. Data is shown for oligonucleotides using sense sequences SEQ ID NOs: 577, 581, 582, 584, 586, 588, 590, 591, 593, 594, 595 and 597, and antisense sequences SEQ ID NOs: 791, 598, 599, 601, 603, 605, 607, 608, 610, 611, 612 and 614, respectively, and having different modification patterns. “*” represents oligonucleotides in which the base of the first nucleotide in the 5′ end of the antisense strand is substituted with a uracil.

FIG. 7 is a schematic showing reduction in serum bile acid concentrations upon CYP27A1 knockdown in a partial bile-duct ligation mouse model.

FIG. 8 is a series of images showing reduction in Sirius Red staining as an indicator of fibrosis in the ligated liver lobe of partial bile-duct ligated mice.

### DETAILED DESCRIPTION OF THE INVENTION

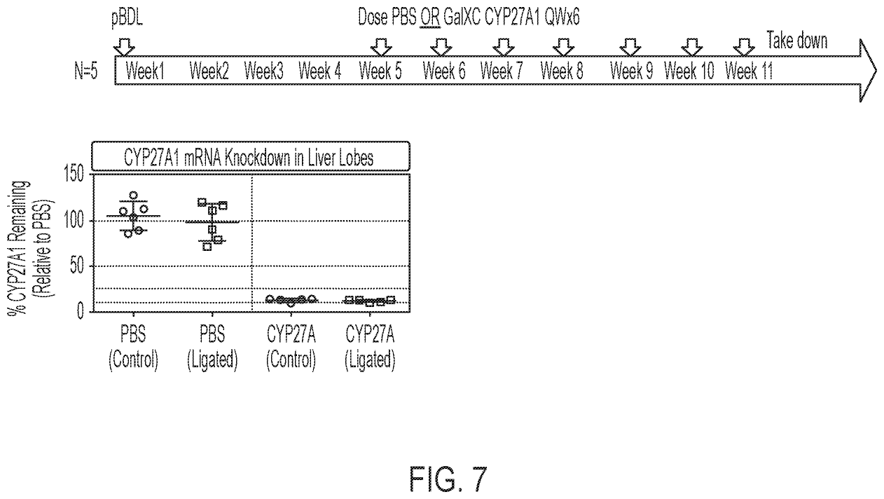

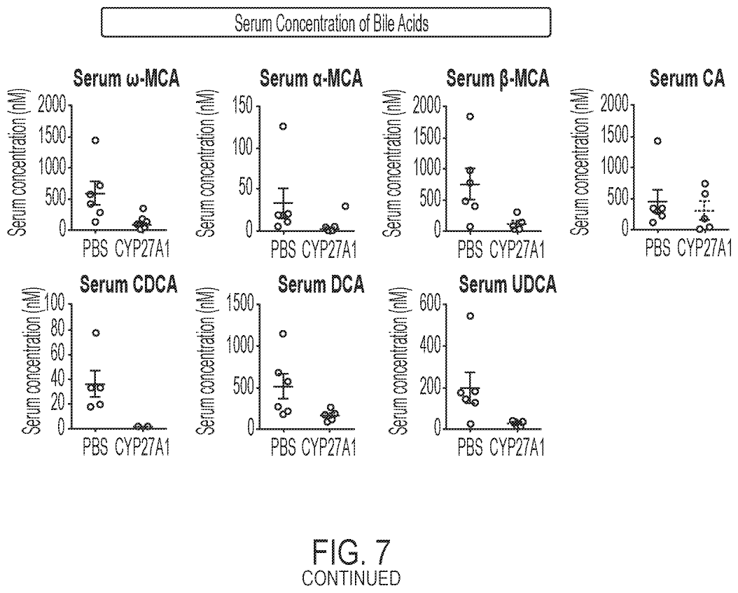

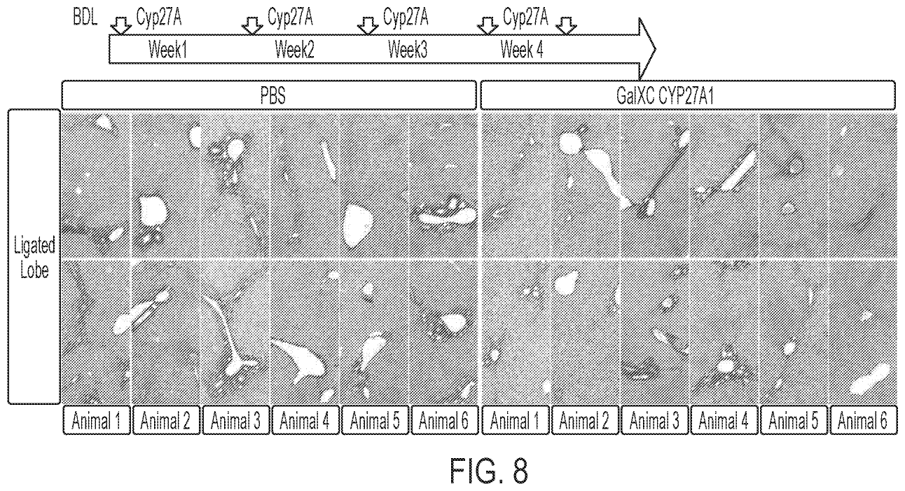

According to some aspects, the disclosure provides oligonucleotides targeting CYP27A1 mRNA that are effective for reducing CYP27A1 expression in cells. These oligonucleotides are useful for the reduction of CYP27A1 in, for example, liver cells (e.g., hepatocytes) for the treatment of bile acid accumulation (e.g., in the context of hepatobiliary disease). Accordingly, in related aspects, the disclosure provides methods of treating bile acid accumulation that involve selectively reducing CYP27A1 gene expression in liver (see, e.g., Example 1 and FIGS. 7 and 8). In certain embodiments, CYP27A1 targeting oligonucleotides provided herein are designed for delivery to selected cells of target tissues (e.g., liver hepatocytes) to treat bile acid accumulation in those tissues.

Further aspects of the disclosure, including a description of defined terms, are provided below.

### I. Definitions

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Administering: As used herein, the terms “administering” or “administration” means to provide a substance (e.g., an oligonucleotide) to a subject in a manner that is pharmacologically useful (e.g., to treat a condition in the subject).

Asialoglycoprotein receptor (ASGPR): As used herein, the term “Asialoglycoprotein receptor” or “ASGPR” refers to a bipartite C-type lectin formed by a major 48 kDa (ASGPR-1) and minor 40 kDa subunit (ASGPR-2). ASGPR is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins).

Attenuates: As used herein, the term “attenuates” means reduces or effectively halts. As a non-limiting example, one or more of the treatments provided herein may reduce or effectively halt the onset or progression of bile acid accumulation in a subject. This attenuation may be exemplified by, for example, a decrease in one or more aspects (e.g., symptoms, tissue characteristics, and cellular, inflammatory or immunological activity, etc.) of bile acid accumulation or symptoms resulting from such accumulation, no detectable progression (worsening) of one or more aspects of bile acid accumulation or symptoms resulting from such accumulation, or no detectable bile acid accumulation or symptoms resulting from such accumulation in a subject when they might otherwise be expected.

Complementary: As used herein, the term “complementary” refers to a structural relationship between nucleotides (e.g., on two nucleotides on opposing nucleic acids or on opposing regions of a single nucleic acid strand) that permits the nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have nucleotide sequences that are complementary to each other so as to form regions of complementarity, as described herein.

CYP27A1: As used herein, the term “CYP27A1” refers to the cytochrome P450 oxidase gene. This gene encodes a protein, cytochrome P450 oxidase, which is a member of the cytochrome P450 superfamily of enzymes, and which is a mitochondrial protein that oxidizes cholesterol intermediates as part of the bile synthesis pathway. Homologs of CYP27A1 are conserved across a range of species including human, mouse, non-human primates, and others (see, e.g., NCBI HomoloGene: 36040). For example, in humans, the CYP27A1 gene encodes multiple transcript variants, including transcript variant 1 (NM_000784.3), and transcript variant 2 (XM_017003488.1). In mice, CYP27A1 encodes multiple transcript variants, namely transcript variant 1 (NM_024264.5) and variant 2 (XM_006495607.2).

Deoxyribonucleotide: As used herein, the term “deoxyribonucleotide” refers to a nucleotide having a hydrogen at the 2′ position of its pentose sugar as compared with a ribonucleotide. A modified deoxyribonucleotide is a deoxyribonucleotide having one or more modifications or substitutions of atoms other than at the 2′ position, including modifications or substitutions in or of the sugar, phosphate group or base.

Double-stranded oligonucleotide: As used herein, the term “double-stranded oligonucleotide” refers to an oligonucleotide that is substantially in a duplex form. In some embodiments, complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from a single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed, e.g., having overhangs at one or both ends. In some embodiments, a double-stranded oligonucleotide comprises antiparallel sequences of nucleotides that are partially complementary, and thus, may have one or more mismatches, which may include internal mismatches or end mismatches.

Duplex: As used herein, the term “duplex,” in reference to nucleic acids (e.g., oligonucleotides), refers to a structure formed through complementary base-pairing of two antiparallel sequences of nucleotides.

Excipient: As used herein, the term “excipient” refers to a non-therapeutic agent that may be included in a composition, for example, to provide or contribute to a desired consistency or stabilizing effect.

Hepatocyte: As used herein, the term “hepatocyte” or “hepatocytes” refers to cells of the parenchymal tissues of the liver. These cells make up approximately 70-85% of the liver's mass and manufacture serum albumin, fibrinogen, and the prothrombin group of dotting factors (except for Factors 3 and 4). Markers for hepatocyte lineage cells may include, but are not limited to: transthyretin (Ttr), glutamine synthetase (Glul), hepatocyte nuclear factor 1a (Hnf1a), and hepatocyte nuclear factor 4a (Hnf4a). Markers for mature hepatocytes may include, but are not limited to: cytochrome P450 (Cyp3a11), fumarylacetoacetate hydrolase (Fah), glucose 6-phosphate (G6p), albumin (Alb), and OC2-2F8. See, e.g., Huch et al., (2013), NATURE, 494(7436): 247-250, the contents of which relating to hepatocyte markers is incorporated herein by reference.

Loop: As used herein, the term “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

Modified Internucleotide Linkage: As used herein, the term “modified internucleotide linkage” refers to an internucleotide linkage having one or more chemical modifications compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

Modified Nucleotide: As used herein, the term “modified nucleotide” refers to a nucleotide having one or more chemical modifications compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modifications in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc. In certain embodiments, a modified nucleotide comprises a 2′-O-methyl or a 2′-F substitution at the 2′ position of the ribose ring.

Nicked Tetraloop Structure: A “nicked tetraloop structure” is a structure of a RNAi oligonucleotide characterized by the presence of separate sense (passenger) and antisense (guide) strands, in which the sense strand has a region of complementarity to the antisense strand such that the two strands form a duplex, and in which at least one of the strands, generally the sense strand, extends from the duplex in which the extension contains a tetraloop and two self-complementary sequences forming a stem region adjacent to the tetraloop, in which the tetraloop is configured to stabilize the adjacent stem region formed by the self-complementary sequences of the at least one strand.

Oligonucleotide: As used herein, the term “oligonucleotide” refers to a short nucleic acid, e.g., of less than 100 nucleotides in length. An oligonucleotide can comprise ribonucleotides, deoxyribonucleotides, and/or modified nucleotides including, for example, modified ribonucleotides. An oligonucleotide may be single-stranded or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, a double-stranded oligonucleotide is an RNAi oligonucleotide.

Overhang: As used herein, the term “overhang” refers to terminal non-base-pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a double-stranded oligonucleotide. In certain embodiments, the overhang is a 3′ or 5′ overhang on the antisense strand or sense strand of a double-stranded oligonucleotide.

Phosphate analog: As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include 5′ phosphonates, such as 5′ methylene phosphonate (5′-MP) and 5′-(E)-vinyl phosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethyl phosphonate, in which the oxygen atom of the oxymethyl group is bound to the sugar moiety (e.g., at its 4′-carbon) or analog thereof. See, for example, International Patent Application PCT/US2017/049909, filed on Sep. 1, 2017, U.S. Provisional Application No. 62/383,207, filed on Sep. 2, 2016, and 62/393,401, filed on Sep. 12, 2016, the contents of each of which relating to phosphate analogs are incorporated herein by reference. Other modifications have been developed for the 5′ end of oligonucleotides (see, e.g., WO 2011/133871; U.S. Pat. No. 8,927,513; and Prakash et al. (2015), Nucleic Acids Res., 43(6):2993-3011, the contents of each of which relating to phosphate analogs are incorporated herein by reference).

Reduced expression: As used herein, the term “reduced expression” of a gene refers to a decrease in the amount of RNA transcript or protein encoded by the gene and/or a decrease in the amount of activity of the gene in a cell or subject, as compared to an appropriate reference cell or subject. For example, the act of treating a cell with a double-stranded oligonucleotide (e.g., one having an antisense strand that is complementary to CYP27A1 mRNA sequence) may result in a decrease in the amount of RNA transcript, protein and/or enzymatic activity (e.g., encoded by the CYP27A1 gene) compared to a cell that is not treated with the double-stranded oligonucleotide. Similarly, “reducing expression” as used herein refers to an act that results in reduced expression of a gene (e.g., CYP27A1).

Region of Complementarity: As used herein, the term “region of complementarity” refers to a sequence of nucleotides of a nucleic acid (e.g., a double-stranded oligonucleotide) that is sufficiently complementary to an antiparallel sequence of nucleotides (e.g., a target nucleotide sequence within an mRNA) to permit hybridization between the two sequences of nucleotides under appropriate hybridization conditions, e.g., in a phosphate buffer, in a cell, etc. A region of complementarity may be fully complementary to a nucleotide sequence (e.g., a target nucleotide sequence present within an mRNA or portion thereof). For example, a region of complementary that is fully complementary to a nucleotide sequence present in an mRNA has a contiguous sequence of nucleotides that is complementary, without any mismatches or gaps, to a corresponding sequence in the mRNA. Alternatively, a region of complementarity may be partially complementary to a nucleotide sequence (e.g., a nucleotide sequence present in an mRNA or portion thereof). For example, a region of complementary that is partially complementary to a nucleotide sequence present in an mRNA has a contiguous sequence of nucleotides that is complementary to a corresponding sequence in the mRNA but that contains one or more mismatches or gaps (e.g., 1, 2, 3, or more mismatches or gaps) compared with the corresponding sequence in the mRNA, provided that the region of complementarity remains capable of hybridizing with the mRNA under appropriate hybridization conditions.

Ribonucleotide: As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

RNAi Oligonucleotide: As used herein, the term “RNAi oligonucleotide” refers to either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA.

Strand: As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

Subject: As used herein, the term “subject” means any mammal, including mice, rabbits, and humans. In one embodiment, the subject is a human or non-human primate. The terms “individual” or “patient” may be used interchangeably with “subject.”

Synthetic: As used herein, the term “synthetic” refers to a nucleic acid or other molecule that is artificially synthesized (e.g., using a machine (e.g., a solid state nucleic acid synthesizer)) or that is otherwise not derived from a natural source (e.g., a cell or organism) that normally produces the molecule.

Targeting ligand: As used herein, the term “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

Tetraloop: As used herein, the term “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. In some embodiments, a tetraloop may stabilize a base pair in an adjacent stem duplex by stacking interactions. In addition, interactions among the nucleotides in a tetraloop include but are not limited to non-Watson-Crick base-pairing, stacking interactions, hydrogen bonding, and contact interactions (Cheong et al., Nature 1990 Aug. 16; 346(6285):680-2; Heus and Pardi, SCIENCE 1991 Jul. 12; 253(5016):191-4). In some embodiments, a tetraloop comprises or consists of 3 to 6 nucleotides, and is typically 4 to 5 nucleotides. In certain embodiments, a tetraloop comprises or consists of three, four, five, or six nucleotides, which may or may not be modified (e.g., which may or may not be conjugated to a targeting moiety). In one embodiment, a tetraloop consists of four nucleotides. Any nucleotide may be used in the tetraloop and standard IUPAC-IUB symbols for such nucleotides may be used as described in Cornish-Bowden (1985) NUCL. ACIDS RES. 13: 3021-3030. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., PROC NATL ACAS SCI USA. 1990 November; 87(21):8467-71; Antao et al., NUCLEIC ACIDS RES. 1991 Nov. 11; 19(21):5901-5). Examples of DNA tetraloops include the d(GNNA) family of tetraloops (e.g., d(GTTA)), the d(GNRA) family of tetraloops, the d(GNAB) family of tetraloops, the d(CNNG) family of tetraloops, and the d(TNCG) family of tetraloops (e.g., d(TTCG)). See, for example: Nakano et al. BIOCHEMISTRY, 41 (48), 14281-14292, 2002. SHINJI et al. NIPPON KAGAKKAI KOEN YOKOSHU VOL. 78th; NO. 2; PAGE. 731 (2000), which are incorporated by reference herein for their relevant disclosures. In some embodiments, the tetraloop is contained within a nicked tetraloop structure.

Treat: As used herein, the term “treat” refers to the act of providing care to a subject in need thereof, e.g., through the administration a therapeutic agent (e.g., an oligonucleotide) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### II. Oligonucleotide-Based Inhibitors

i. CYP27A1 Targeting Oligonucleotides

Potent oligonucleotides have been identified herein through examination of the CYP27A1 mRNA, including mRNAs of multiple different species (human, rhesus monkey, and mouse (see, e.g., Example 1)) and in vitro and in vivo testing. Such oligonucleotides can be used to achieve therapeutic benefit for subjects experiencing bile acid accumulation and/or having liver hepatobiliary disease by reducing CYP27A1 activity, and consequently, by decreasing bile acid levels and/or liver fibrosis. For example, potent RNAi oligonucleotides are provided herein that have a sense strand comprising, or consisting of, a sequence as set forth in any one of SEQ ID NO: 577-578, 581-597, 759-762, 785, and 787 and an antisense strand comprising, or consisting of, a complementary sequence selected from any one of SEQ ID NO: 579-580, 598-614, 763-766, 786, and 788, as is also arranged the table provided in Appendix A (e.g., a sense strand comprising a sequence as set forth in SEQ ID NO: 577 and an antisense strand comprising a sequence as set forth in SEQ ID NO: 579). The sequences can be put into multiple different structures (or formats), as described herein.

In some embodiments, it has been discovered that certain regions of CYP27A1 mRNA are hotspots for targeting because they are more amenable than other regions to oligonucleotide-based inhibition. In some embodiments, a hotspot region of CYP27A1 consists of a sequence as forth in any one of SEQ ID NOs: 767-781. These regions of CYP27A1 mRNA may be targeted using oligonucleotides as discussed herein for purposes of inhibiting CYP27A1 mRNA expression.

Accordingly, in some embodiments, oligonucleotides provided herein are designed so as to have regions of complementarity to CYP27A1 mRNA (e.g., within a hotspot of CYP27A1 mRNA) for purposes of targeting the mRNA in cells and inhibiting its expression. The region of complementarity is generally of a suitable length and base content to enable annealing of the oligonucleotide (or a strand thereof) to CYP27A1 mRNA for purposes of inhibiting its expression.

In some embodiments, an oligonucleotide disclosed herein comprises a region of complementarity (e.g., on an antisense strand of a double-stranded oligonucleotide) that is at least partially complementary to a sequence as set forth in any one of SEQ ID NOs: 1-288, 615-686 and 789, which include sequences mapping to within hotspot regions of CYP27A1 mRNA. In some embodiments, an oligonucleotide disclosed herein comprises a region of complementarity (e.g., on an antisense strand of a double-stranded oligonucleotide) that is fully complementary to a sequence as set forth in any one of SEQ ID NOs: 1-288, 615-686 and 789. In some embodiments, a region of complementarity of an oligonucleotide that is complementary to contiguous nucleotides of a sequence as set forth in any one of SEQ ID NOs: 1-288, 615-686 and 789 spans the entire length of an antisense strand. In some embodiments, a region of complementarity of an oligonucleotide that is complementary to contiguous nucleotides of a sequence as set forth in any one of SEQ ID NOs:1-288, 615-686 and 789 spans a portion of the entire length of an antisense strand (e.g., all but two nucleotides at the 3′ end of the antisense strand). In some embodiments, an oligonucleotide disclosed herein comprises a region of complementarity (e.g., on an antisense strand of a double-stranded oligonucleotide) that is at least partially (e.g., fully) complementary to a contiguous stretch of nucleotides spanning nucleotides 1-19 of a sequence as set forth in any one of SEQ ID NOs:577-578, 581-597, and 759-762, 785, and 787.

In some embodiments, the region of complementarity is at least 12, at least 13, at least 14, at least 15, at least 16, at least 17, at least 18, at least 19, at least 20, at least 21, at least 22, at least 23, at least 24, or at least 25 nucleotides in length. In some embodiments, an oligonucleotide provided herein has a region of complementarity to CYP27A1 mRNA that is in the range of 12 to 30 (e.g., 12 to 30, 12 to 22, 15 to 25, 17 to 21, 18 to 27, 19 to 27, or 15 to 30) nucleotides in length. In some embodiments, an oligonucleotide provided herein has a region of complementarity to CYP27A1 mRNA that is 12, 13, 14, 15, 16, 17, 18, 19, 20, 21, 22, 23, 24, 25, 26, 27, 28, 29, or 30 nucleotides in length.

In some embodiments, a region of complementarity to CYP27A1 mRNA may have one or more mismatches compared with a corresponding sequence of CYP27A1 mRNA. A region of complementarity on an oligonucleotide may have up to 1, up to 2, up to 3, up to 4, etc. mismatches provided that it maintains the ability to form complementary base pairs with CYP27A1 mRNA under appropriate hybridization conditions. Alternatively, a region of complementarity on an oligonucleotide may have no more than 1, no more than 2, no more than 3, or no more than 4 mismatches provided that it maintains the ability to form complementary base pairs with CYP27A1 mRNA under appropriate hybridization conditions. In some embodiments, if there are more than one mismatches in a region of complementarity, they may be positioned consecutively (e.g., 2, 3, 4, or more in a row), or interspersed throughout the region of complementarity provided that the oligonucleotide maintains the ability to form complementary base pairs with CYP27A1 mRNA under appropriate hybridization conditions.

Still, in some embodiments, double-stranded oligonucleotides provided herein comprise, of consist of, a sense strand having a sequence as set forth in any one of SEQ ID NO: 1-288, 615-686 and 789 and an antisense strand comprising a complementary sequence selected from SEQ ID NO: 289-576, as is arranged in the table provided in Appendix A (e.g., a sense strand comprising a sequence as set forth in SEQ ID NO: 1 and an antisense strand comprising a sequence as set forth in SEQ ID NO: 289).

ii. Oligonucleotide Structures

There are a variety of structures of oligonucleotides that are useful for targeting CYP27A1 mRNA in the methods of the present disclosure, including RNAi, miRNA, etc. Any of the structures described herein or elsewhere may be used as a framework to incorporate or target a sequence described herein (e.g., a hotpot sequence of CYP27A1 such as those illustrated in SEQ ID NOs: 767-781). Double-stranded oligonucleotides for targeting CYP27A1 expression (e.g., via the RNAi pathway) generally have a sense strand and an antisense strand that form a duplex with one another. In some embodiments, the sense and antisense strands are not covalently linked. However, in some embodiments, the sense and antisense strands are covalently linked.

In some embodiments, double-stranded oligonucleotides for reducing CYP27A1 expression engage RNA interference (RNAi). For example, RNAi oligonucleotides have been developed with each strand having sizes of 19-25 nucleotides with at least one 3′ overhang of 1 to 5 nucleotides (see, e.g., U.S. Pat. No. 8,372,968). Longer oligonucleotides have also been developed that are processed by the Dicer enzyme to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as WO2010033225, which are incorporated by reference herein for their disclosure of these oligonucleotides). Such structures may include single-stranded extensions (on one or both sides of the molecule) as well as double-stranded extensions.

In some embodiments, sequences described herein can be incorporated into, or targeted using, oligonucleotides that comprise separate sense and antisense strands that are both in the range of 17 to 36 nucleotides in length. In some embodiments, oligonucleotides incorporating such sequences are provided that have a tetraloop structure within a 3′ extension of their sense strand, and two terminal overhang nucleotides at the 3′ end of the separate antisense strand. In some embodiments, the two terminal overhang nucleotides are GG. Typically, one or both of the two terminal GG nucleotides of the antisense strand is or are not complementary to the target.

In some embodiments, oligonucleotides incorporating such sequences are provided that have sense and antisense strands that are both in the range of 21 to 23 nucleotides in length. In some embodiments, a 3′ overhang is provided on the sense, antisense, or both sense and antisense strands that is 1 or 2 nucleotides in length. In some embodiments, an oligonucleotide has a guide strand of 23 nucleotides and a passenger strand of 21 nucleotides, in which the 3′-end of passenger strand and 5′-end of guide strand form a blunt end and where the guide strand has a two nucleotide 3′ overhang.

In some embodiments, oligonucleotides may be in the range of 21 to 23 nucleotides in length. In some embodiments, oligonucleotides may have an overhang (e.g., of 1, 2, or 3 nucleotides in length) in the 3′ end of the sense and/or antisense strands. In some embodiments, oligonucleotides (e.g., siRNAs) may comprise a 21 nucleotide guide strand that is antisense to a target RNA and a complementary passenger strand, in which both strands anneal to form a 19-bp duplex and 2 nucleotide overhangs at either or both 3′ ends. See, for example, U.S. Pat. Nos. 9,012,138, 9,012,621, and 9,193,753, the contents of each of which are incorporated herein for their relevant disclosures. In some embodiments, an oligonucleotide of the invention has a 36 nucleotide sense strand that comprises a region extending beyond the antisense-sense duplex, where the extension region has a stem-tetraloop structure where the stem is a six base pair duplex and where the tetraloop has four nucleotides. In certain of those embodiments, three or four of the tetraloop nucleotides are each conjugated to a monovalent GalNac ligand.

In some embodiments, an oligonucleotide of the invention comprises a 25 nucleotide sense strand and a 27 nucleotide antisense strand that when acted upon by a dicer enzyme results in an antisense strand that is incorporated into the mature RISC.

Other oligonucleotides designs for use with the compositions and methods disclosed herein include: 16-mer siRNAs (see, e.g., Nucleic Acids in Chemistry and Biology. Blackburn (ed.), Royal Society of Chemistry, 2006), shRNAs (e.g., having 19 bp or shorter stems; see, e.g., Moore et al. Methods Mol. Biol. 2010; 629:141-158), blunt siRNAs (e.g., of 19 bps in length; see: e.g., Kraynack and Baker, RNA Vol. 12, p 163-176 (2006)), asymmetrical siRNAs (aiRNA; see, e.g., Sun et al., NAT. BIOTECHNOL. 26, 1379-1382 (2008)), asymmetric shorter-duplex siRNA (see, e.g., Chang et al., MOL THER. 2009 April; 17(4): 725-32), fork siRNAs (see, e.g., Hohjoh, FEBS LETTERS, Vol 557, issues 1-3; January 2004, p 193-198), single-stranded siRNAs (Elsner; NATURE BIOTECHNOLOGY 30, 1063 (2012)), dumbbell-shaped circular siRNAs (see, e.g., Abe et al. J AM CHEM SOC 129: 15108-15109 (2007)), and small internally segmented interfering RNA (sisiRNA; see, e.g., Bramsen et al., NUCLEIC ACIDS RES. 2007 September; 35(17): 5886-5897). Each of the foregoing references is incorporated by reference in its entirety for the related disclosures therein. Further non-limiting examples of an oligonucleotide structures that may be used in some embodiments to reduce or inhibit the expression of CYP27A1 are microRNA (miRNA), short hairpin RNA (shRNA), and short siRNA (see, e.g., Hamilton et al., EMBO J., 2002, 21(17): 4671-4679; see also U.S. Application No. 20090099115).

a. Antisense Strands

In some embodiments, an oligonucleotide disclosed herein for targeting CYP27A1 comprises an antisense strand comprising or consisting of a sequence as set forth in any one of SEQ ID NOs: 289-576, 687-758, and 790 or 579-580, 598-614, 763-766, 786, 788, and 792. In some embodiments, an oligonucleotide comprises an antisense strand comprising or consisting of at least 12 (e.g., at least 12, at least 13, at least 14, at least 15, at least 16, at least 17, at least 18, at least 19, at least 20, at least 21, at least 22, or at least 23) contiguous nucleotides of a sequence as set forth in any one of SEQ ID NOs: 289-576, 687-758, and 790 or 579-580, 598-614, 763-766, 786, 788, and 792.

In some embodiments, a double-stranded oligonucleotide may have an antisense strand of up to 40 nucleotides in length (e.g., up to 40, up to 35, up to 30, up to 27, up to 25, up to 21, up to 19, up to 17, or up to 12 nucleotides in length). In some embodiments, an oligonucleotide may have an antisense strand of at least 12 nucleotides in length (e.g., at least 12, at least 15, at least 19, at least 21, at least 22, at least 25, at least 27, at least 30, at least 35, or at least 38 nucleotides in length). In some embodiments, an oligonucleotide may have an antisense strand in a range of 12 to 40 (e.g., 12 to 40, 12 to 36, 12 to 32, 12 to 28, 15 to 40, 15 to 36, 15 to 32, 15 to 28, 17 to 22, 17 to 25, 19 to 27, 19 to 30, 20 to 40, 22 to 40, 25 to 40, or 32 to 40) nucleotides in length. In some embodiments, an oligonucleotide may have an antisense strand of 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, an antisense strand of an oligonucleotide may be referred to as a “guide strand.” For example, if an antisense strand can engage with RNA-induced silencing complex (RISC) and bind to an Argonaut protein, or engage with or bind to one or more similar factors, and direct silencing of a target gene, it may be referred to as a guide strand. In some embodiments, a sense strand complementary to a guide strand may be referred to as a “passenger strand.”

b. Sense Strands

In some embodiments, an oligonucleotide disclosed herein for targeting CYP27A1 comprises or consists of a sense strand sequence as set forth in in any one of SEQ ID NOs: 1-288, 615-686 and 789 or 577-578, 581-597, 759-762, 785, and 787. In some embodiments, an oligonucleotide has a sense strand that comprises or consists of at least 12 (e.g., at least 13, at least 14, at least 15, at least 16, at least 17, at least 18, at least 19, at least 20, at least 21, at least 22, or at least 23) contiguous nucleotides of a sequence as set forth in in any one of SEQ ID NOs: 1-288, 615-686 and 789 or 577-578, 581-597, 759-762, 785, and 787.

In some embodiments, an oligonucleotide may have a sense strand (or passenger strand) of up to 40 nucleotides in length (e.g., up to 40, up to 36, up to 30, up to 27, up to 25, up to 21, up to 19, up to 17, or up to 12 nucleotides in length). In some embodiments, an oligonucleotide may have a sense strand of at least 12 nucleotides in length (e.g., at least 12, at least 15, at least 19, at least 21, at least 25, at least 27, at least 30, at least 36, or at least 38 nucleotides in length). In some embodiments, an oligonucleotide may have a sense strand in a range of 12 to 40 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40, or 32 to 40) nucleotides in length. In some embodiments, an oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

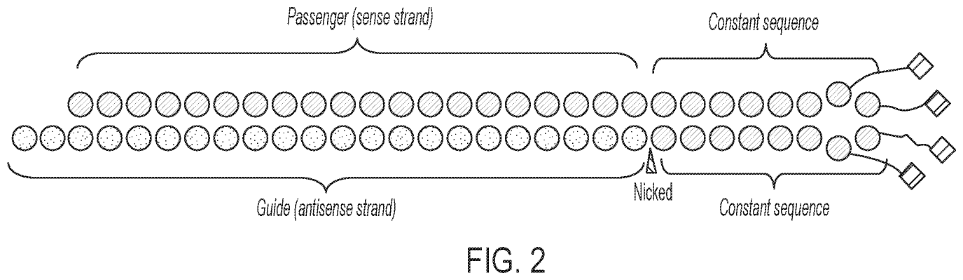

In some embodiments, a sense strand comprises a stem-loop structure at its 3′-end. In some embodiments, a sense strand comprises a stem-loop structure at its 5′-end. In some embodiments, a stem is a duplex of 2, 3, 4, 5, 6, 7, 8, 9, 10, 11, 12, 13, or 14 base pairs in length. In some embodiments, a stem-loop provides the molecule better protection against degradation (e.g., enzymatic degradation) and facilitates targeting characteristics for delivery to a target cell. For example, in some embodiments, a loop provides added nucleotides on which modification can be made without substantially affecting the gene expression inhibition activity of an oligonucleotide. In certain embodiments, an oligonucleotide is provided herein in which the sense strand comprises (e.g., at its 3′-end) a stem-loop set forth as: S₁-L-S₂, in which S₁ is complementary to S₂, and in which L forms a loop between S₁ and S₂ of up to 10 nucleotides in length (e.g., 3, 4, 5, 6, 7, 8, 9, or 10 nucleotides in length). FIG. 2 depicts a non-limiting example of such an oligonucleotide.

In some embodiments, a loop (L) of a stem-loop is a tetraloop (e.g., within a nicked tetraloop structure). A tetraloop may contain ribonucleotides, deoxyribonucleotides, modified nucleotides, and combinations thereof. Typically, a tetraloop has 4 to 5 nucleotides.

c. Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 20, 21, 22, 23, 24, 25, 26, 27, 28, 29, or 30 nucleotides in length. In some embodiments a duplex formed between a sense and antisense strand does not span the entire length of the sense strand and/or antisense strand. In some embodiments, a duplex between a sense and antisense strand spans the entire length of either the sense or antisense strands. In certain embodiments, a duplex between a sense and antisense strand spans the entire length of both the sense strand and the antisense strand.

d. Oligonucleotide Ends

In some embodiments, an oligonucleotide provided herein comprises sense and antisense strands, such that there is a 3′-overhang on either the sense strand or the antisense strand, or both the sense and antisense strand. In some embodiments, oligonucleotides provided herein have one 5′end that is thermodynamically less stable compared to the other 5′ end. In some embodiments, an asymmetric oligonucleotide is provided that includes a blunt end at the 3′ end of a sense strand and an overhang at the 3′ end of an antisense strand. In some embodiments, a 3′ overhang on an antisense strand is 1-8 nucleotides in length (e.g., 1, 2, 3, 4, 5, 6, 7 or 8 nucleotides in length).

Typically, an oligonucleotide for RNAi has a two nucleotide overhang on the 3′ end of the antisense (guide) strand. However, other overhangs are possible. In some embodiments, an overhang is a 3′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides. However, in some embodiments, the overhang is a 5′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides.

In some embodiments, one or more (e.g., 2, 3, 4) terminal nucleotides of the 3′ end or 5′ end of a sense and/or antisense strand are modified. For example, in some embodiments, one or two terminal nucleotides of the 3′ end of an antisense strand are modified. In some embodiments, the last nucleotide at the 3′ end of an antisense strand is modified, e.g., comprises 2′-modification, e.g., a 2′-O-methoxyethyl. In some embodiments, the last one or two terminal nucleotides at the 3′ end of an antisense strand are complementary to the target. In some embodiments, the last one or two nucleotides at the 3′ end of the antisense strand are not complementary to the target. In some embodiments, the 5′ end and/or the 3′ end of a sense or antisense strand has an inverted cap nucleotide.

e. Mismatches

In some embodiments, there is one or more (e.g., 1, 2, 3, or 4) mismatches between a sense and antisense strand. If there is more than one mismatch between a sense and antisense strand, they may be positioned consecutively (e.g., 2, 3 or more in a row), or interspersed throughout the region of complementarity. In some embodiments, the 3′-terminus of the sense strand contains one or more mismatches. In one embodiment, two mismatches are incorporated at the 3′ terminus of the sense strand. In some embodiments, base mismatches or destabilization of segments at the 3′-end of the sense strand of the oligonucleotide improved the potency of synthetic duplexes in RNAi, possibly through facilitating processing by Dicer.

iii. Single-Stranded Oligonucleotides

In some embodiments, an oligonucleotide for reducing CYP27A1 expression as described herein is single-stranded. Such structures may include, but are not limited to single-stranded RNAi oligonucleotides. Recent efforts have demonstrated the activity of single-stranded RNAi oligonucleotides (see, e.g., Matsui et al. (May 2016), Molecular Therapy, Vol. 24(5), 946-955). However, in some embodiments, oligonucleotides provided herein are antisense oligonucleotides (ASOs). An antisense oligonucleotide is a single-stranded oligonucleotide that has a nucleobase sequence which, when written in the 5′ to 3′ direction, comprises the reverse complement of a targeted segment of a particular nucleic acid and is suitably modified (e.g., as a gapmer) so as to induce RNaseH mediated cleavage of its target RNA in cells or (e.g., as a mixmer) so as to inhibit translation of the target mRNA in cells. Antisense oligonucleotides for use in the instant disclosure may be modified in any suitable manner known in the art including, for example, as shown in U.S. Pat. No. 9,567,587, which is incorporated by reference herein for its disclosure regarding modification of antisense oligonucleotides (including, e.g., length, sugar moieties of the nucleobase (pyrimidine, purine), and alterations of the heterocyclic portion of the nucleobase). Further, antisense molecules have been used for decades to reduce expression of specific target genes (see, e.g., Bennett et al.; PHARMACOLOGY OF ANTISENSE DRUGS, ANNUAL REVIEW OF PHARMACOLOGY AND TOXICOLOGY, Vol. 57: 81-105).

iv. Oligonucleotide Modifications

Oligonucleotides may be modified in various ways to improve or control specificity, stability, delivery, bioavailability, resistance from nuclease degradation, immunogenicity, base-paring properties, RNA distribution and cellular uptake and other features relevant to therapeutic or research use. See, e.g., Bramsen et al., Nucleic Acids Res., 2009, 37, 2867-2881; Bramsen and Kjems (FRONTIERS IN GENETICS, 3 (2012): 1-22). Accordingly, in some embodiments, oligonucleotides of the present disclosure may include one or more suitable modifications. In some embodiments, a modified nucleotide has a modification in its base (or nucleobase), the sugar (e.g., ribose, deoxyribose), or the phosphate group.

The number of modifications on an oligonucleotide and the positions of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier (e.g., “naked delivery”), it may be advantageous for at least some of the its nucleotides to be modified. Accordingly, in certain embodiments of any of the oligonucleotides provided herein, all or substantially all of the nucleotides of an oligonucleotide are modified. In certain embodiments, more than half of the nucleotides are modified. In certain embodiments, less than half of the nucleotides are modified. Typically, with naked delivery, every nucleotide is modified at the 2′-position of the sugar group of that nucleotide. These modifications may be reversible or irreversible. Typically, the 2′ position modification is a 2′-fluoro, 2′-O-methyl, etc. In some embodiments, an oligonucleotide as disclosed herein has a number and type of modified nucleotides sufficient to cause the desired characteristic (e.g., protection from enzymatic degradation, capacity to target a desired cell after in vivo administration, and/or thermodynamic stability).

a. Sugar Modifications

In some embodiments, a modified sugar (also referred to herein as a sugar analog) includes a modified deoxyribose or ribose moiety, e.g., in which one or more modifications occur at the 2′, 3′, 4′, and/or 5′ carbon position of the sugar. In some embodiments, a modified sugar may also include non-natural alternative carbon structures such as those present in locked nucleic acids (“LNA”) (see, e.g., Koshkin et al. (1998), TETRAHEDRON 54, 3607-3630), unlocked nucleic acids (“UNA”) (see, e.g., Snead et al. (2013), MOLECULAR THERAPY—NUCLEIC ACIDS, 2, e103), and bridged nucleic acids (“BNA”) (see, e.g., Imanishi and Obika (2002), The Royal Society of Chemistry, CHEM. COMMUN., 1653-1659). Koshkin et al., Snead et al., and Imanishi and Obika are incorporated by reference herein for their disclosures relating to sugar modifications.

In some embodiments, a nucleotide modification in a sugar comprises a 2′-modification. In some embodiments, the 2′-modification may be 2′-aminoethyl, 2′-fluoro, 2′-O-methyl, 2′-O-methoxyethyl, or 2′-deoxy-2′-fluoro-β-d-arabinonucleic acid. Typically, the modification is 2′-fluoro, 2′-O-methyl, or 2′-O-methoxyethyl. However, a large variety of 2′ position modifications that have been developed for use in oligonucleotides can be employed in oligonucleotides disclosed herein. See, e.g., Bramsen et al., Nucleic Acids Res., 2009, 37, 2867-2881. In some embodiments, a modification in a sugar comprises a modification of the sugar ring, which may comprise modification of one or more carbons of the sugar ring. For example, a modification of a sugar of a nucleotide may comprise a linkage between the 2′-carbon and a 1′-carbon or 4′-carbon of the sugar. For example, the linkage may comprise an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, the terminal 3′-end group (e.g., a 3′-hydroxyl) is a phosphate group or other group, which can be used, for example, to attach linkers, adapters or labels or for the direct ligation of an oligonucleotide to another nucleic acid.

b. 5′ Terminal Phosphates

5′-terminal phosphate groups of oligonucleotides may or in some circumstances enhance the interaction with Argonaut 2. However, oligonucleotides comprising a 5′-phosphate group may be susceptible to degradation via phosphatases or other enzymes, which can limit their bioavailability in vivo. In some embodiments, oligonucleotides include analogs of 5′ phosphates that are resistant to such degradation. In some embodiments, a phosphate analog may be oxymethylphosphonate, vinylphosphonate, or malonylphosphonate. In certain embodiments, the 5′ end of an oligonucleotide strand is attached to a chemical moiety that mimics the electrostatic and steric properties of a natural 5′-phosphate group (“phosphate mimic”) (see, e.g., Prakash et al. (2015), Nucleic Acids Res., Nucleic Acids Res. 2015 Mar. 31; 43(6): 2993-3011, the contents of which relating to phosphate analogs are incorporated herein by reference). Many phosphate mimics have been developed that can be attached to the 5′ end (see, e.g., U.S. Pat. No. 8,927,513, the contents of which relating to phosphate analogs are incorporated herein by reference). Other modifications have been developed for the 5′ end of oligonucleotides (see, e.g., WO 2011/133871, the contents of which relating to phosphate analogs are incorporated herein by reference). In certain embodiments, a hydroxyl group is attached to the 5′ end of the oligonucleotide.

In some embodiments, an oligonucleotide has a phosphate analog at a 4′-carbon position of the sugar (referred to as a “4′-phosphate analog”). See, for example, International Patent Application PCT/US2017/049909, filed on Sep. 1, 2017, U.S. Provisional Application No. 62/383,207, entitled 4′-Phosphate Analogs and Oligonucleotides Comprising the Same, filed on Sep. 2, 2016, and 62/393,401, filed on Sep. 12, 2016, entitled 4′-Phosphate Analogs and Oligonucleotides Comprising the Same, the contents of each of which relating to phosphate analogs are incorporated herein by reference. In some embodiments, an oligonucleotide provided herein comprises a 4′-phosphate analog at a 5′-terminal nucleotide. In some embodiments, a phosphate analog is an oxymethyl phosphonate, in which the oxygen atom of the oxymethyl group is bound to the sugar moiety (e.g., at its 4′-carbon) or analog thereof. In other embodiments, a 4′-phosphate analog is a thiomethyl phosphonate or an aminomethyl phosphonate, in which the sulfur atom of the thiomethyl group or the nitrogen atom of the aminomethyl group is bound to the 4′-carbon of the sugar moiety or analog thereof. In certain embodiments, a 4′-phosphate analog is an oxymethylphosphonate. In some embodiments, an oxymethyl phosphonate is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si(CH₃)₃, or a protecting group. In certain embodiments, the alkyl group is CH₂CH₃. More typically, R is independently selected from H, CH₃, or CH₂CH₃.

c. Modified Internucleoside Linkages

In some embodiments, the oligonucleotide may comprise a modified internucleoside linkage. In some embodiments, phosphate modifications or substitutions may result in an oligonucleotide that comprises at least one (e.g., at least 1, at least 2, at least 3, at least 4, or at least 5) modified internucleotide linkage. In some embodiments, any one of the oligonucleotides disclosed herein comprises 1 to 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9, or 10 modified internucleotide linkages.

A modified internucleotide linkage may be a phosphorodithioate linkage, a phosphorothioate linkage, a phosphotriester linkage, a thionoalkylphosphonate linkage, a thionoalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

d. Base Modifications

In some embodiments, oligonucleotides provided herein have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain a nitrogen atom. See e.g., U.S. Published Patent Application No. 20080274462. In some embodiments, a modified nucleotide comprises a universal base. However, in certain embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution that, when present in a duplex, can be positioned opposite more than one type of base without substantially altering the structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include inosine, 1-β-D-ribofuranosyl-5-nitroindole, and/or 1-β-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside. Nucleic Acids Res. 1995 Nov. 11; 23(21):4363-70; Loakes et al., 3-Nitropyrrole and 5-nitroindole as universal bases in primers for DNA sequencing and PCR. NUCLEIC ACIDS RES. 1995 Jul. 11; 23(13):2361-6; Loakes and Brown, 5-Nitroindole as an universal base analogue. NUCLEIC ACIDS RES. 1994 Oct. 11; 22(20):4039-43. Each of the foregoing is incorporated by reference herein for their disclosures relating to base modifications).

e. Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance. See U.S. Published Application No. 2011/0294869 originally assigned to Traversa Therapeutics, Inc. (“Traversa”), PCT Publication No. WO 2015/188197 to Solstice Biologics, Ltd. (“Solstice”), Meade et al., NATURE BIOTECHNOLOGY, 2014, 32:1256-1263 (“Meade”), PCT Publication No. WO 2014/088920 to Merck Sharp & Dohme Corp, each of which are incorporated by reference for their disclosures of such modifications. This reversible modification of the internucleotide diphosphate linkages is designed to be cleaved intracellularly by the reducing environment of the cytosol (e.g. glutathione). Earlier examples include neutralizing phosphotriester modifications that were reported to be cleavable inside cells (Dellinger et al. J. AM. CHEM. SOC. 2003, 125:940-950).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed and the result is a cleaved oligonucleotide. Using reversible, glutathione sensitive moieties, it is possible to introduce sterically larger chemical groups into the oligonucleotide of interest as compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′-carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of a sugar, particularly when the modified nucleotide is the 3′-terminal nucleotide of the oligonucleotide. In some embodiments, the glutathione-sensitive moiety comprises a sulfonyl group. See, e.g., International Patent Application PCT/US2017/048239 and U.S. Prov. Appl. No. 62/378,635, entitled Compositions Comprising Reversibly Modified Oligonucleotides and Uses Thereof, which was filed on Aug. 23, 2016, the contents of which are incorporated by reference herein for its relevant disclosures.

v. Targeting Ligands

In some embodiments, it may be desirable to target the oligonucleotides of the disclosure to one or more cells or one or more organs. Such a strategy may help to avoid undesirable effects in other organs, or may avoid undue loss of the oligonucleotide to cells, tissue or organs that would not benefit for the oligonucleotide. Accordingly, in some embodiments, oligonucleotides disclosed herein may be modified to facilitate targeting of a particular tissue, cell or organ, e.g., to facilitate delivery of the oligonucleotide to the liver. In certain embodiments, oligonucleotides disclosed herein may be modified to facilitate delivery of the oligonucleotide to the hepatocytes of the liver. In some embodiments, an oligonucleotide comprises a nucleotide that is conjugated to one or more targeting ligands.

A targeting ligand may comprise a carbohydrate, amino sugar, cholesterol, peptide, polypeptide, protein or part of a protein (e.g., an antibody or antibody fragment) or lipid. In some embodiments, a targeting ligand is an aptamer. For example, a targeting ligand may be an RGD peptide that is used to target tumor vasculature or glioma cells, CREKA peptide to target tumor vasculature or stoma, transferrin, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand, as described, for example, in International Patent Application Publication WO 2016/100401, which was published on Jun. 23, 2016, the relevant contents of which are incorporated herein by reference.

In some embodiments, it is desirable to target an oligonucleotide that reduces the expression of CYP27A1 to the hepatocytes of the liver of a subject. Any suitable hepatocyte targeting moiety may be used for this purpose.

GalNAc is a high affinity ligand for asialoglycoprotein receptor (ASGPR), which is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins). Conjugation (either indirect or direct) of GalNAc moieties to oligonucleotides of the instant disclosure may be used to target these oligonucleotides to the ASGPR expressed on these hepatocyte cells.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3, or 4 monovalent GalNAc moieties, and is typically conjugated to 3 or 4 monovalent GalNAc moieties). In some embodiments, an oligonucleotide of the instant disclosure is conjugated to one or more bivalent GalNAc, trivalent GalNAc, or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of the loop (L) of the stem-loop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a GalNAc moiety. In some embodiments, GalNAc moieties are conjugated to a nucleotide of the sense strand. For example, four GalNAc moieties can be conjugated to nucleotides in the tetraloop of the sense strand, where each GalNAc moiety is conjugated to one nucleotide.

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in International Patent Application Publication Number WO2016100401 A1, which published on Jun. 23, 2016, and the contents of which relating to such linkers are incorporated herein by reference. In some embodiments, the linker is a labile linker. However, in other embodiments, the linker is fairly stable. In some embodiments, a duplex extension (up to 3, 4, 5, or 6 base pairs in length) is provided between a targeting ligand (e.g., a GalNAc moiety) and a double-stranded oligonucleotide.

### III. Formulations

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., single-stranded or double-stranded oligonucleotides) to reduce the expression of CYP27A1. Such compositions can be suitably formulated such that when administered to a subject, either into the immediate environment of a target cell or systemically, a sufficient portion of the oligonucleotides enter the cell to reduce CYP27A1 expression. Any of a variety of suitable oligonucleotide formulations can be used to deliver oligonucleotides for the reduction of CYP27A1 as disclosed herein. In some embodiments, an oligonucleotide is formulated in buffer solutions such as phosphate-buffered saline solutions, liposomes, micellar structures, and capsids.

In some embodiments, naked oligonucleotides or conjugates thereof are formulated in water or in an aqueous solution (e.g., water with pH adjustments). In some embodiments, naked oligonucleotides or conjugates thereof are formulated in basic buffered aqueous solutions (e.g., PBS). Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine) can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere, or a nanoparticle, or may be otherwise formulated for administration to the cells, tissues, organs, or body of a subject in need thereof (see, e.g., Remington: The Science and Practice of Pharmacy, 22nd edition, Pharmaceutical Press, 2013).

In some embodiments, formulations as disclosed herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Typically. the route of administration is intravenous or subcutaneous.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. In many cases, it will be preferable to include isotonic agents, for example, sugars, polyalcohols such as mannitol, sorbitol, and sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., an oligonucleotide for reducing CYP27A1 expression) or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though a number of embodiments are directed to liver-targeted delivery of any of the oligonucleotides disclosed herein, targeting of other tissues is also contemplated.

### IV. Methods of Use

i. Reducing CYP27A1 Expression in Cells

In some embodiments, methods are provided for delivering to a cell an effective amount any one of oligonucleotides disclosed herein for purposes of reducing expression of CYP27A1 in the cell. Methods provided herein are useful in any appropriate cell type. In some embodiments, a cell is any cell that expresses CYP27A1 (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue and skin). In some embodiments, the cell is a primary cell that has been obtained from a subject and that may have undergone a limited number of a passages, such that the cell substantially maintains its natural phenotypic properties. In some embodiments, a cell to which the oligonucleotide is delivered is ex vivo or in vitro (i.e., can be delivered to a cell in culture or to an organism in which the cell resides). In specific embodiments, methods are provided for delivering to a cell an effective amount any one of the oligonucleotides disclosed herein for purposes of reducing expression of CYP27A1 solely or primarily in hepatocytes.

In some embodiments, oligonucleotides disclosed herein can be introduced using appropriate nucleic acid delivery methods including injection of a solution containing the oligonucleotides, bombardment by particles covered by the oligonucleotides, exposing the cell or organism to a solution containing the oligonucleotides, or electroporation of cell membranes in the presence of the oligonucleotides. Other appropriate methods for delivering oligonucleotides to cells may be used, such as lipid-mediated carrier transport, chemical-mediated transport, and cationic liposome transfection such as calcium phosphate, and others.

The consequences of inhibition can be confirmed by an appropriate assay to evaluate one or more properties of a cell or subject, or by biochemical techniques that evaluate molecules indicative of CYP27A1 expression (e.g., RNA, protein). In some embodiments, the extent to which an oligonucleotide provided herein reduces levels of expression of CYP27A1 is evaluated by comparing expression levels (e.g., mRNA or protein levels of CYP27A1 to an appropriate control (e.g., a level of CYP27A1 expression in a cell or population of cells to which an oligonucleotide has not been delivered or to which a negative control has been delivered). In some embodiments, an appropriate control level of CYP27A1 expression may be a predetermined level or value, such that a control level need not be measured every time. The predetermined level or value can take a variety of forms. In some embodiments, a predetermined level or value can be single cut-off value, such as a median or mean.

In some embodiments, administration of an oligonucleotide as described herein results in a reduction in the level of CYP27A1 expression in a cell. In some embodiments, the reduction in levels of CYP27A1 expression may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of CYP27A1. The appropriate control level may be a level of CYP27A1 expression in a cell or population of cells that has not been contacted with an oligonucleotide as described herein. In some embodiments, the effect of delivery of an oligonucleotide to a cell according to a method disclosed herein is assessed after a finite period of time. For example, levels of CYP27A1 may be analyzed in a cell at least 8 hours, 12 hours, 18 hours, 24 hours; or at least one, two, three, four, five, six, seven, or fourteen days after introduction of the oligonucleotide into the cell.

In some embodiments, an oligonucleotide is delivered in the form of a transgene that is engineered to express in a cell the oligonucleotides (e.g., its sense and antisense strands). In some embodiments, an oligonucleotide is delivered using a transgene that is engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). In some embodiments, transgenes can be injected directly to a subject.

ii. Treatment Methods

Aspects of the disclosure relate to methods for reducing CYP27A1 expression for the treatment of liver fibrosis, e.g., associated with bile acid accumulation in the context of hepatobiliary disease. In some embodiments, the methods may comprise administering to a subject in need thereof an effective amount of any one of the oligonucleotides disclosed herein. Such treatments could be used, for example, to a subject at risk of (or susceptible to) liver fibrosis and/or hepatobiliary disease.

In certain aspects, the disclosure provides a method for preventing in a subject, a disease or disorder as described herein by administering to the subject a therapeutic agent (e.g., an oligonucleotide or vector or transgene encoding same). In some embodiments, the subject to be treated is a subject who will benefit therapeutically from a reduction in the amount of CYP27A1 protein, e.g., in the liver.

Methods described herein typically involve administering to a subject an effective amount of an oligonucleotide, that is, an amount capable of producing a desirable therapeutic result. A therapeutically acceptable amount may be an amount that is capable of treating a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions disclosed herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intramuscular injection,), topically (e.g., epicutaneous, inhalational, via eye drops, or through a mucous membrane), or by direct injection into a target organ (e.g., the liver of a subject). Typically, oligonucleotides disclosed herein are administered intravenously or subcutaneously.

In some embodiments, oligonucleotides are administered at a dose in a range of 0.1 mg/kg to 25 mg/kg (e.g., 1 mg/kg to 5 mg/kg). In some embodiments, oligonucleotides are administered at a dose in a range of 0.1 mg/kg to 5 mg/kg or in a range of 0.5 mg/kg to 5 mg/kg.

As a non-limiting set of examples, the oligonucleotides of the instant disclosure would typically be administered once per year, twice per year, quarterly (once every three months), bi-monthly (once every two months), monthly, or weekly.

In some embodiments, the subject to be treated is a human (e.g., a human patient) or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### EXAMPLES

### Example 1: Development of CYP27A1 Oligonucleotide Inhibitors Using Human and Mouse Cell-Based Assays

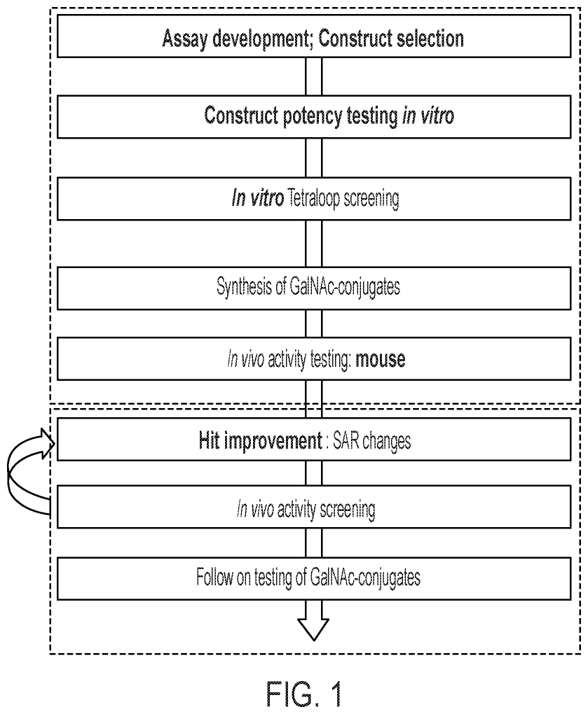

FIG. 1 shows workflows using human and mouse-based assays to develop candidate oligonucleotides for inhibition of CYP27A1 expression. First, a computer-based algorithm was used to generate candidate oligonucleotide sequences for CYP27A1 inhibition. Cell-based assays and PCR assays were then employed for evaluation of candidate oligonucleotides for their ability to reduce CYP27A1 expression.

The computer algorithm provided 2114 oligonucleotides that were complementary to the human CYP27A1 mRNA (SEQ ID NO: 782, Table 1), of which 1084 were also complementary to the rhesus CYP27A1 mRNA (SEQ ID NO: 783, Table 1), and 24 were also complementary to the mouse CYP27A1 mRNA (SEQ ID NO: 784, Table 1). 8 oligonucleotides were complementary to human, mouse and rhesus CYP27A1 mRNA. Examples of CYP27A1 mRNA sequences are outlined in Table 1:

TABLE 1
Sequences of human, rhesus monkey and mouse CYP27A1 mRNA

| Species | GenBank RefSeq # | Sequence Identifier |
|---|---|---|
| Human | NM_000784.3 | SEQ ID NO: 782 |
| Rhesus | NM_001194021.1 | SEQ ID NO: 783 |
| Monkey | | |
| Mouse | NM_024264.5 | SEQ ID NO: 784 |

Of the 2114 oligonucleotides that the algorithm provided, 288 oligonucleotides were selected as candidates for experimental evaluation in a HepG2 cell-based assay. In this assay, cells expressing CYP27A1 were transfected with the oligonucleotides. Cells were maintained for a period of time following transfection and then levels of remaining CYP27A1 mRNA were interrogated using SYBR®-based qPCR assays. Two qPCR assays, a 3′ assay and a 5′ assay were used. All 288 oligonucleotides had the same modification pattern, designated M15, which contains a combination of ribonucleotides, deoxyribonucleotides and 2′-O-methyl modified nucleotides. The sequences of the oligonucleotides tested are provided in Table 2.

TABLE 2
Candidate Oligonucleotide Sequences for Human Cell-Based Assay

| | | | Sense | Antisense |
|---|---|---|---|---|
| Hs | Rh | Mm | SEQ ID NO | SEQ ID NO |
| X | X | | 1 to 58; | 289-346; |
| | | | 78 to153; | 366 to 441; |
| | | | 155 to 159; | 443 to 447; |
| | | | 193; 194; | 481; 482; |
| | | | 199 to 250; | 487 to 538; |
| | | | 252 to 282 | 540 to 570 |
| X | | | 59 to 77; | 347 to 365; |
| | | | 154; | 442; |
| | | | 160 to 170; | 448 to 458; |
| | | | 197; 198; | 485; 486; |
| | | | 251; | 539; |
| | | | 283 to 288 | 571 to 576 |
| X | X | X | 171 to 178 | 459 to 466 |
| X | | X | 179 to 192; | 467 to 480; |
| | | | 195; 196 | 483; 484 |

Hs: human, Rh: rhesus monkey, and Mm: mouse; the sense and antisense SEQ ID NO columns provide the sense strand and respective antisense strand that are hybridized to make each oligonucleotide. For example, sense strand with SEQ ID NO: 1 hybridizes with antisense strand with SEQ ID NO: 289; each of the oligonucleotides tested had the same modification pattern.

Hotspots in CYP27A1 mRNA

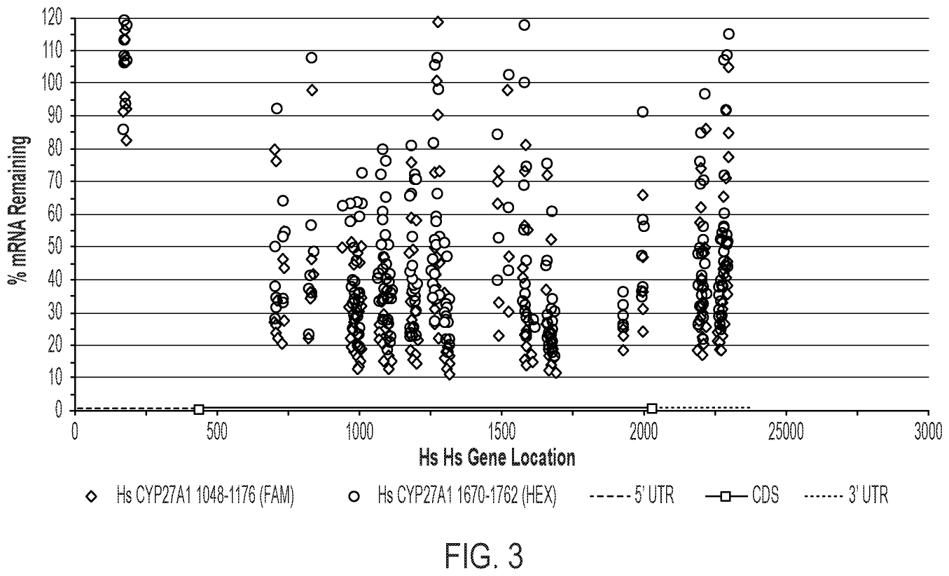

Data from the screen of the 288 candidate oligonucleotides is shown in FIG. 3. Oligonucleotides are arranged based on the location of complementarity to the human (Hs) gene location. Oligonucleotides resulting in less than or equal to 25% mRNA remaining compared to negative controls were considered hits. Three oligonucleotides that were not found to inhibit CYP27A1 expression were used as negative controls. In addition, transfection of cells with house-keeping gene Hypoxanthine-guanine phosphoribosyltransferase (HPRT) was used as a positive control for transfection.

119 hits were identified based on this criteria. Based on the activity of and locations these oligonucleotides (FIG. 3), hotspots on the human CYP27A1 mRNA were defined. A hotspot was identified as a stretch on the human CYP27A1 mRNA sequence associated with at least one oligonucleotide resulting in mRNA levels that were less than or equal to 25% in either assay compared with controls. These hotspots can be visualized in FIG. 3. Accordingly, the following hotspots within the human CYP27A1 mRNA sequence were identified: 699-711, 729-735, 822-836, 970-1009, 1065-1088, 1095-1112, 1181-1203, 1297-1317, 1488-1492, 1591-1616, 1659-1687, 1929-1932, 1995-2001, 2204-2225, and 2262-2274. The sequences of the hotspots are outlined in Table 3.

TABLE 3
Sequences of Hotspots

| | | SEQ |
|---|---|---|
| Hotspot | | ID |
| Position | Sequence | NO: |
| 699-711 | CUGCACCAGUUACAGGUGCUUUACAAGGCCAAGUAC | 767 |
| | G | |
| 729-735 | AAGUACGGUCCAAUGUGGAUGUCCUACUUAG | 768 |
| 822-836 | GGCAAGUACCCAGUACGGAACGACAUGGAGCUAUGG | 769 |
| | AAG | |
| 970-1009 | CAGCGCUCUAUACGGAUGCUUUCAAUGAGGUGAUUG | 770 |
| | AUGACUUUAUGACUCGACUGGACCAGCU | |
| 1065-1088 | UCGGACAUGGCUCAACUCUUCUACUACUUUGCCUUG | 771 |
| | GAAGCUAUUUGC | |
| 1095-1112 | GCCUUGGAAGCUAUUUGCUACAUCCUGUUCGAGAAA | 772 |
| | CGCAUU | |
| 1181-1203 | CAGAUCCAUCGGGUUAAUGUUCCAGAACUCACUCUA | 773 |
| | UGCCACCUUCC | |
| 1297-1317 | CCUUUGGGAAGAAGCUGAUUGAUGAGAAGCUCGAAG | 774 |
| | AUAUGGAGG | |
| 1488-1492 | CUGACAUGGGCCCUGUACCACCUCUCAAA | 775 |
| 1591-1616 | AGGACUUUGCCCACAUGCCGUUGCAAAGCUGUGCUU | 776 |
| | AAGGAGACUCUG | |
| 1659-1687 | CCCACAAACUCCCGGAUCAUAGAAAAGGAAAUUGAA | 777 |
| | GUUGAUGGCUUCCUCUU | |
| 1929-1932 | GCAAGGCUGAUCCAGAAGUACAAGGUGG | 778 |
| 1995-2001 | CGCAUUGUCCUGGUUCCCAAUAAGAAAGUGG | 779 |
| 2204-2225 | UUUGCCACUUCUAUCAUUUUUGAGCAACUCCCUCUCA | 780 |
| | GCUAAAAGG | |
| 2262-2274 | CGCAUUGCUGUCCUUGGGUAGAAUAUAAAAUAAAGG | 781 |
| | G | |

### Dose Response Analysis

Based on gene location and sequence conservation between species, of the 119 oligonucleotides found to be most active in the first screen, 96 oligonucleotides were subjected to a secondary screen. In this secondary screen, the oligonucleotides were tested using the same assay as in the primary screen, but at three different concentrations (1 nM, 0.1 nM and 0.01 nM). Oligonucleotides showing activity at two more concentrations were selected for further analysis.

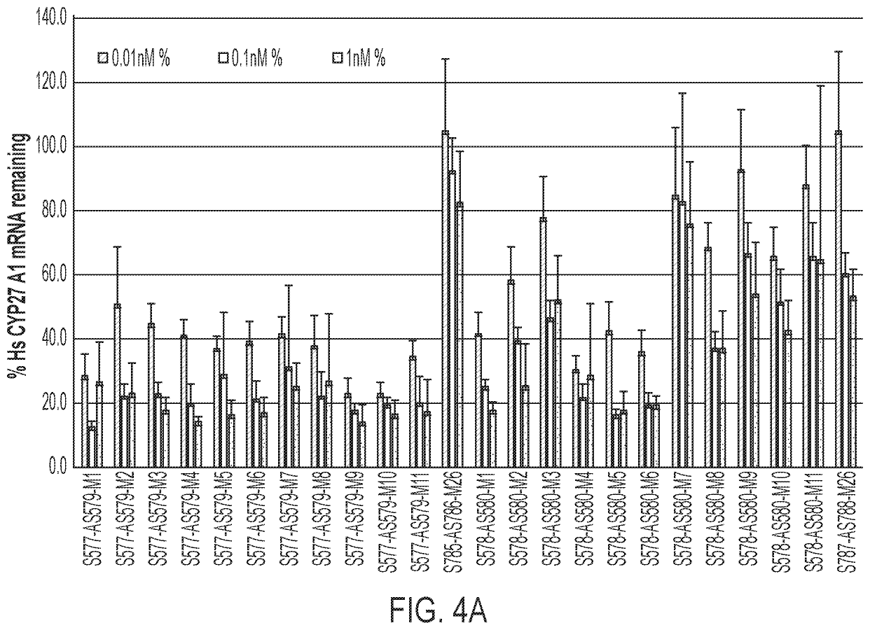

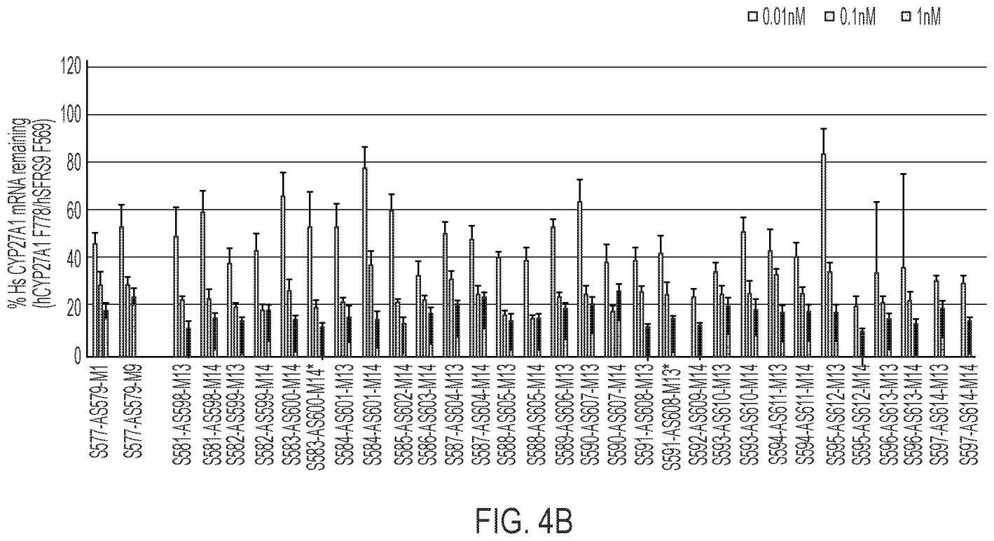

At this stage, select oligonucleotides were modified to contain tetraloops and adapt different modification patterns. Stem-loop sequences were incorporated at the 3′-end of the sense (passenger) strand, in which the loop sequence was that of a tetraloop. Thus, the molecules were converted to nicked tetraloop structures (a 36-mer passenger strand with a 22-mer guide strand). See FIG. 2 for a generic tetraloop structure. These were then tested at three different concentrations (0.01 nM, 0.1 nM and 1 nM) for their ability to reduce CYP27A1 mRNA expression. FIG. 4A shows data for oligonucleotides made from two base sequences with tetraloops, each adapted to 10 different modification patterns, designated M1 to M12. For this experiment, two oligonucleotides (i.e., 5785-AS786-M26 and 5787-AS788-M26) were are 21-mers instead of being 22-mers were also tested. 5785-AS786-M26 and 5787-AS788-M26 are 21-mer versions of 5577-AS579-M26 and 5578-AS580-M26, respectively. These were tested because a Dicer enzyme may cleave a larger oligonucleotide into a 21-mer or a 22-mer. FIG. 4B shows similar data, but for 16 base sequences with tetraloops, each adapted to 1 or 2 different modification patterns, designated M13 and M14. Oligonucleotides 5577-AS579-M1 and 5577-AS579-M9 were used as inter-experiment calibrators in the experiments resulting in data shown in FIGS. 4A and 4B. Additionally, in oligonucleotides depicted by “*” in FIG. 4B, the base of the first nucleotide in the 5′ end of the antisense strand is substituted with a uracil to improve activity.

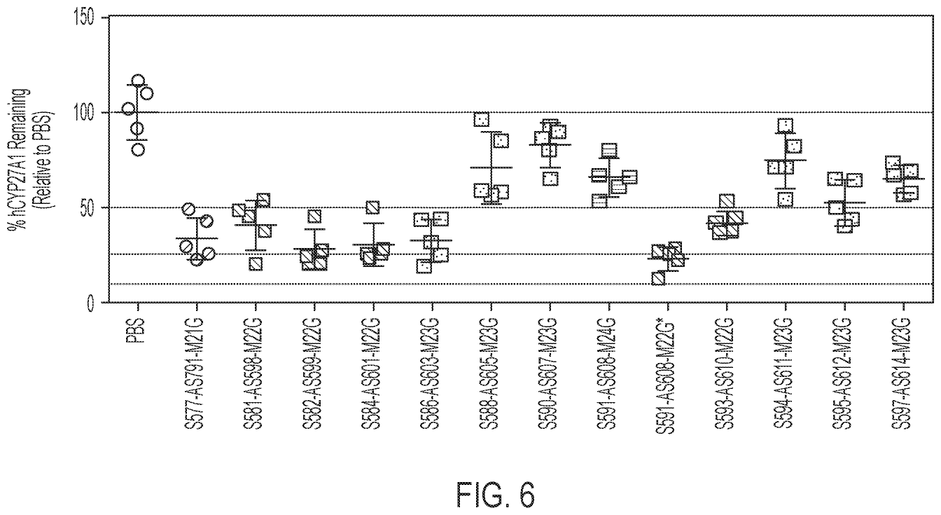

Data from these experiments were assessed to identify tetraloops and modification patterns that would improve delivery properties, but maintain activity for reduction of CYP27A1 expression. Based on this analysis, select oligonucleotides were then conjugated to GalNAc moieties and assayed (FIG. 6). For the oligonucleotides shown in FIG. 6, four GalNAc moieties were conjugated to nucleotides in the tetraloop of the sense strand. Conjugation was done using a click linker. The GalNAc used was as shown below:

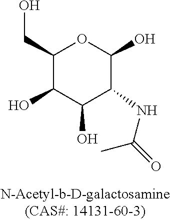

The ability of oligonucleotides to reduce CYP27A1 expression was influenced by modification patterns. For example, oligonucleotides S591-AS608-M24G and S591-AS608-M22G are different only in that S591-AS608-M24G contains a cytosine at position 1 and a natural 5′ phosphate on the antisense stand, whereas S591-AS608-M22G contains a uracil at position 1 and a 5′ phosphate analog on the antisense stand.

Protein levels of CYP27A1 were also assessed along with mRNA levels.

### Testing Murine Models

In parallel with the experiments using human HepG2 cells, oligonucleotides were also screened in AML12 murine cells. 96 oligonucleotides that were complementary to mouse CYP27A1 mRNA (SEQ ID NO: 784) were tested. Cells expressing CYP27A1 were transfected with the oligonucleotides and levels of remaining CYP27A1 mRNA were interrogated using SYBR®-based qPCR assays. Table 4 outlines the sequences of oligonucleotides that were tested.

TABLE 4
Candidate Oligonucleotide Sequences for Murine Cell-Based
Assay: Hs: human, Rh: rhesus monkey, and Mm: mouse; the sense
and antisense SEQ ID NO columns provide the sense strand
and respective antisense strand (listed in order relative
to one another) that are annealed to make each oligonucleotide.
For example, sense strand with SEQ ID NO: 1 hybridizes with
antisense strand with SEQ ID NO: 289; each of the oligonucleotides
tested had the same modification pattern.

| | | | Sense | Antisense |
|---|---|---|---|---|
| Hs | Rh | Mm | SEQ ID NO | SEQ ID NO |
| | | X | 615 to 686 | 687 to 758 |
| X | X | X | 171 to 178 | 459 to 466 |
| X | | X | 179 to 196 | 467 to 484 |

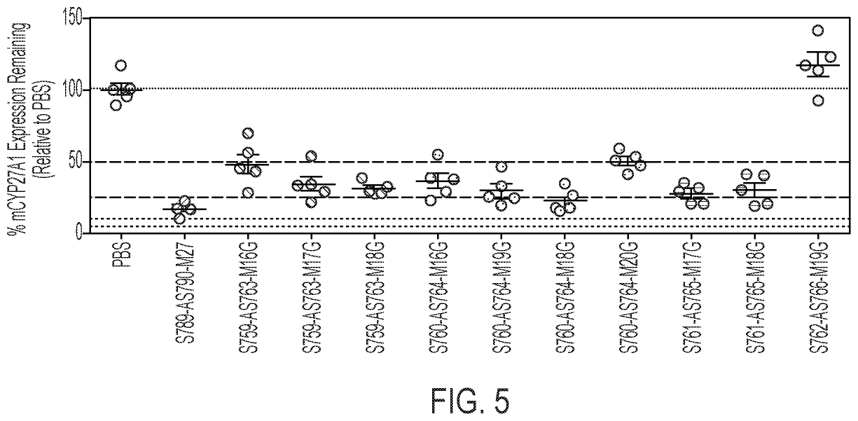

Using similar criteria as in the human cell-based assays, 26 of these were then subjected to screening at multiple concentrations. Different modification patterns were then applied to 8 of the 26 oligonucleotides. Based on their activity, 4 sequences with varying modification patterns were conjugated to GalNAc moieties. FIG. 5 shows activity of these GalNAc-conjugated oligonucleotides with tetraloops. For the oligonucleotides shown in FIG. 5, four GalNAc moieties were conjugated to nucleotides in the tetraloop of the sense strand. Select oligonucleotides were subjected to testing in a partial bile-duct ligation mouse model. In this experiment, a parent oligonucleotide (i.e., a 25/27-mer) that was formulated in a lipid nanoparticle, 5789-AS790-M27 was used as a control. This oligonucleotide was not conjugated to GalNAc moieties.

The left liver lobe bile duct was surgically ligated in female CD-1 mice, while the bile ducts supplying the other lobes were left untreated. Four weeks after surgery, the mice were subcutaneously injected with either PBS or GalXC-CYP27A1 conjugates (i.e. GalNAc-conjugated oligonucleotides) at 10 mg/kg every week for 4 more weeks. At the end of the study, the mice were sacrificed and serum and liver tissue was collected. RNA was purified from the livers to generate cDNA. CYP27A1 mRNA levels were then estimated by qPCR using mouse specific CYP27A1 primer/probes. Serum bile acid concentrations were measured by LC-MS with heavy isotope labeled bile acid standards. CYP27A1 knockdown significantly decreased the concentrations of bile acids in circulation (FIG. 7).

The left liver lobe bile duct was surgically ligated in female CD-1 mice, while the bile ducts supplying the other lobes were left untreated. After recovery from surgery, the mice were subcutaneously injected with either PBS or GalXC-CYP27A1 conjugates at 10 mg/kg every week for 4 weeks. At the end of the study, the mice were sacrificed and their livers were collected. Liver sections were then stained with Sirius Red, a dye that specifically stains fibrotic regions in the liver. CYP27A1 knockdown decreases the amount of fibrosis as measured by Sirius Red staining (FIG. 8).

### Materials and Methods

### Transfection

For the first screen, Lipofectamine RNAiMAX™ was used to complex the oligonucleotides for efficient transfection. Oligonucleotides, RNAiMAX and Opti-MEM were added to a plate and incubated at room temperature for 20 minutes prior to transfection. Media was aspirated from a flask of actively passaging cells and the cells are incubated at 37° C. in the presence of trypsin for 3-5 minutes. After cells no longer adhered to the flask, cell growth media (lacking penicillin and streptomycin) was added to neutralize the trypsin and to suspend the cells. A 10 μL aliquot was removed and counted with a hemocytometer to quantify the cells on a per millimeter basis. For HeLa cells, 20,000 cells were seeded per well in 100 μL of media. The suspension was diluted with the known cell concentration to obtain the total volume required for the number of cells to be transfected. The diluted cell suspension was added to the 96 well transfection plates, which already contained the oligonucleotides in Opti-MEM. The transfection plates were then incubated for 24 hours at 37° C. After 24 hours of incubation, media was aspirated from each well. Cells were lysed using the lysis buffer from the Promega RNA Isolation kit. The lysis buffer was added to each well. The lysed cells were then transferred to the Corbett XtractorGENE (QIAxtractor) for RNA isolation or stored at −80° C.

For subsequent screens and experiments, e.g., the secondary screen, Lipofectamine RNAiMAx was used to complex the oligonucleotides for reverse transfection. The complexes were made by mixing RNAiMAX and siRNAs in OptiMEM medium for 15 minutes. The transfection mixture was transferred to multi-well plates and cell suspension was added to the wells. After 24 hours incubation the cells were washed once with PBS and then lysed using lysis buffer from the Promega SV96 kit. The RNA was purified using the SV96 plates in a vacuum manifold. Four microliters of the purified RNA was then heated at 65° C. for 5 minutes and cooled to 4° C. The RNA was then used for reverse transcription using the High Capacity Reverse Transcription kit (Life Technologies) in a 10 microliter reaction. The cDNA was then diluted to 50 μL with nuclease free water and used for quantitative PCR with multiplexed 5′-endonuclease assays and SSoFast qPCR mastermix (Bio-Rad laboratories).

cDNA Synthesis

RNA was isolated from mammalian cells in tissue culture using the Corbett X-tractor Gene™ (QIAxtractor). A modified SuperScript II protocol was used to synthesize cDNA from the isolated RNA. Isolated RNA (approximately 5 ng/4) was heated to 65° C. for five minutes and incubated with dNPs, random hexamers, oligo dTs, and water. The mixture was cooled for 15 seconds. An “enzyme mix,” consisting of water, 5× first strand buffer, DTT, SUPERase⋅In™ (an RNA inhibitor), and SuperScript II RTase was added to the mixture. The contents were heated to 42° C. for one hour, then to 70° C. for 15 minutes, and then cooled to 4° C. using a thermocycler. The resulting cDNA was then subjected to SYBR®-based qPCR. The qPCR reactions were multiplexed, containing two 5′ endonuclease assays per reaction.

qPCR Assays

Primer sets were initially screened using SYBR®-based qPCR. Assay specificity was verified by assessing melt curves as well as “minus RT” controls. Dilutions of cDNA template (10-fold serial dilutions from 20 ng and to 0.02 ng per reaction) from HeLa and Hepa1-6 cells are used to test human (Hs) and mouse (Mm) assays, respectively. qPCR assays were set up in 384-well plates, covered with MicroAmp film, and run on the 7900HT from Applied Biosystems. Reagent concentrations and cycling conditions included the following: 2×SYBR mix, 10 μM forward primer, 10 μM reverse primer, DD H₂O, and cDNA template up to a total volume of 10 pt.

### Cloning

PCR amplicons that displayed a single melt-curve were ligated into the pGEM®-T Easy vector kit from Promega according to the manufacturer's instructions. Following the manufacturer's protocol, JM109 High Efficiency cells were transformed with the newly ligated vectors. The cells were then plated on LB plates containing ampicillin and incubated at 37° C. overnight for colony growth.

### PCR Screening and Plasmid Mini-Prep

PCR was used to identify colonies of E. coli that had been transformed with a vector containing the ligated amplicon of interest. Vector-specific primers that flank the insert were used in the PCR reaction. All PCR products were then run on a 1% agarose gel and imaged by a transilluminator following staining. Gels were assessed qualitatively to determine which plasmids appeared to contain a ligated amplicon of the expected size (approximately 300 bp, including the amplicon and the flanking vector sequences specific to the primers used).

The colonies that were confirmed transformants by PCR screening were then incubated overnight in cultures consisting of 2 mL LB broth with ampicillin at 37° C. with shaking. E. coli cells were then lysed, and the plasmids of interest were isolated using Promega's Mini-Prep kit. Plasmid concentration was determined by UV absorbance at 260 nm.

### Plasmid Sequencing and Quantification

Purified plasmids were sequenced using the BigDye® Terminator sequencing kit. The vector-specific primer, T7, was used to give read lengths that span the insert. The following reagents were used in the sequencing reactions: water, 5× sequencing buffer, BigDye terminator mix, T7 primer, and plasmid (100 ng/4) to a volume of 10 μL. The mixture was held at 96° C. for one minute, then subjected to 15 cycles of 96° C. for 10 seconds, 50° C. for 5 seconds, 60° C. for 1 minute, 15 seconds; 5 cycles of 96° C. for 10 seconds, 50° C. for 5 seconds, 60° C. for 1 minute, 30 seconds; and 5 cycles of 96° C. for 10 seconds, 50° C. for 5 seconds, and 60° C. for 2 minutes. Dye termination reactions were then sequenced using Applied Biosystems' capillary electrophoresis sequencers.

Sequence-verified plasmids were then quantified. They were linearized using a single cutting restriction endonuclease. Linearity was confirmed using agarose gel electrophoresis. All plasmid dilutions were made in TE buffer (pH 7.5) with 100 μg of tRNA per mL buffer to reduce non-specific binding of plasmid to the polypropylene vials.

The linearized plasmids were then serially diluted from 1,000,000 to 01 copies per μL and subjected to qPCR. Assay efficiency was calculated and the assays were deemed acceptable if the efficiency was in the range of 90-110%.

### Multi-Plexing Assays

For each target, mRNA levels were quantified by two 5′ nuclease assays. In general, several assays are screened for each target. The two assays selected displayed a combination of good efficiency, low limit of detection, and broad 5′→3′ coverage of the gene of interest (GOI). Both assays against one GOI could be combined in one reaction when different fluorophores were used on the respective probes. Thus, the final step in assay validation was to determine the efficiency of the selected assays when they were combined in the same qPCR or “multi-plexed”.

Linearized plasmids for both assays in 10-fold dilutions were combined and qPCR was performed. The efficiency of each assay was determined as described above. The accepted efficiency rate was 90-110%.

While validating multi-plexed reactions using linearized plasmid standards, C_{q} values for the target of interest were also assessed using cDNA as the template. For human or mouse targets, HeLa and Hepa1-6 cDNA were used, respectively. The cDNA, in this case, was derived from RNA isolated on the Corbett (˜5 ng/μl in water) from untransfected cells. In this way, the observed C_{q} values from this sample cDNA were representative of the expected C_{q} values from a 96-well plate transfection. In cases where C_{q} values were greater than 30, other cell lines were sought that exhibit higher expression levels of the gene of interest. A library of total RNA isolated from via high-throughput methods on the Corbett from each human and mouse line was generated and used to screen for acceptable levels of target expression.

### Description of Oligonucleotide Nomenclature

All oligonucleotides described herein are designated either SN₁-ASN₂-MN₃. The following designations apply:
N₁: sequence identifier number of the sense strand sequence
N₂: sequence identifier number of the antisense strand sequence
N₃: reference number of modification pattern, in which each number represents a pattern of modified nucleotides in the oligonucleotide.
   For example, S27-AS123-M15 represents an oligonucleotide with a sense sequence that is set forth by SEQ ID NO: 27, an antisense sequence that is set forth by SEQ ID NO: 123, and which is adapted to modification pattern number 15.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize or be able to ascertain using no more than routine experimentation, many equivalents to the specific embodiments of the invention described herein. Such equivalents are intended to be encompassed by the following claims.

APPENDIX A

| Oligonucleotide | | S SEQ | | AS SEQ |
|---|---|---|---|---|
| Name | Sense Sequence/mRNA seq | ID NO | Antisense Sequence | ID NO |
| S1-AS289- | CCAGAGUUCAGACCAAGCGAAAAGT | 1 | ACUUUUCGCUUGGUCUG | 289 |
| M15 | | | AACUCUGGGC | |
| S2-AS290- | CAGAGUUCAGACCAAGCGAAAAGTT | 2 | AACUUUUCGCUUGGUCU | 290 |
| M15 | | | GAACUCUGGG | |
| S3-AS291- | AGAGUUCAGACCAAGCGAAAAGUTA | 3 | UAACUUUUCGCUUGGUC | 291 |
| M15 | | | UGAACUCUGG | |
| S4-AS292- | GAGUUCAGACCAAGCGAAAAGUUAT | 4 | AUAACUUUUCGCUUGGU | 292 |
| M15 | | | CUGAACUCUG | |
| S5-AS293- | AGUUCAGACCAAGCGAAAAGUUATT | 5 | AAUAACUUUUCGCUUGG | 293 |
| M15 | | | UCUGAACUCU | |
| S6-AS294- | GUUCAGACCAAGCGAAAAGUUAUTT | 6 | AAAUAACUUUUCGCUUG | 294 |
| M15 | | | GUCUGAACUC | |
| S7-AS295- | UUCAGACCAAGCGAAAAGUUAUUTG | 7 | CAAAUAACUUUUCGCUU | 295 |
| M15 | | | GGUCUGAACU | |
| S8-AS296- | UCAGACCAAGCGAAAAGUUAUUUGA | 8 | UCAAAUAACUUUUCGCU | 296 |
| M15 | | | UGGUCUGAAC | |
| S9-AS297- | CAGACCAAGCGAAAAGUUAUUUGAG | 9 | CUCAAAUAACUUUUCGCU | 297 |
| M15 | | | UGGUCUGAA | |
| S10-AS298- | AGACCAAGCGAAAAGUUAUUUGAGA | 10 | UCUCAAAUAACUUUUCGC | 298 |
| M15 | | | UUGGUCUGA | |
| S11-AS299- | GACCAAGCGAAAAGUUAUUUGAGAG | 11 | CUCUCAAAUAACUUUUCG | 299 |
| M15 | | | CUUGGUCUG | |
| S12-AS300- | ACCAAGCGAAAAGUUAUUUGAGAGG | 12 | CCUCUCAAAUAACUUUUC | 300 |
| M15 | | | GCUUGGUCU | |
| S13-AS301- | CCAAGCGAAAAGUUAUUUGAGAGGC | 13 | GCCUCUCAAAUAACUUUU | 301 |
| M15 | | | CGCUUGGUC | |
| S14-AS302- | CUGCACCAGUUACAGGUGCUUUACA | 14 | UGUAAAGCACCUGUAACU | 302 |
| M15 | | | GGUGCAGUU | |
| S15-AS303- | UGCACCAGUUACAGGUGCUUUACAA | 15 | UUGUAAAGCACCUGUAAC | 303 |
| M15 | | | UGGUGCAGU | |
| S16-AS304- | GCACCAGUUACAGGUGCUUUACAAG | 16 | CUUGUAAAGCACCUGUAA | 304 |
| M15 | | | CUGGUGCAG | |
| S17-AS305- | CACCAGUUACAGGUGCUUUACAAGG | 17 | CCUUGUAAAGCACCUGUA | 305 |
| M15 | | | ACUGGUGCA | |
| S18-AS306- | CCAGUUACAGGUGCUUUACAAGGCC | 18 | GGCCUUGUAAAGCACCUG | 306 |
| M15 | | | UAACUGGUG | |
| S19-AS307- | CAGUUACAGGUGCUUUACAAGGCCA | 19 | UGGCCUUGUAAAGCACCU | 307 |
| M15 | | | GUAACUGGU | |
| S20-AS308- | AGUUACAGGUGCUUUACAAGGCCAA | 20 | UUGGCCUUGUAAAGCACC | 308 |
| M15 | | | UGUAACUGG | |
| S21-AS309- | UACAGGUGCUUUACAAGGCCAAGTA | 21 | UACUUGGCCUUGUAAAG | 309 |
| M15 | | | CACCUGUAAC | |
| S22-AS310- | ACAGGUGCUUUACAAGGCCAAGUAC | 22 | GUACUUGGCCUUGUAAA | 310 |
| M15 | | | GCACCUGUAA | |
| S23-AS311- | CAGGUGCUUUACAAGGCCAAGUACG | 23 | CGUACUUGGCCUUGUAA | 311 |
| M15 | | | AGCACCUGUA | |
| S24-AS312- | AAGUACGGUCCAAUGUGGAUGUCCT | 24 | AGGACAUCCACAUUGGAC | 312 |
| M15 | | | CGUACUUGG | |
| S25-AS313- | GUACGGUCCAAUGUGGAUGUCCUAC | 25 | GUAGGACAUCCACAUUG | 313 |
| M15 | | | GACCGUACUU | |
| S26-AS314- | ACGGUCCAAUGUGGAUGUCCUACTT | 26 | AAGUAGGACAUCCACAUU | 314 |
| M15 | | | GGACCGUAC | |
| S27-AS315- | CGGUCCAAUGUGGAUGUCCUACUTA | 27 | UAAGUAGGACAUCCACAU | 315 |
| M15 | | | UGGACCGUA | |
| S28-AS316- | GGUCCAAUGUGGAUGUCCUACUUAG | 28 | CUAAGUAGGACAUCCACA | 316 |
| M15 | | | UUGGACCGU | |
| S29-AS317- | GGCAAGUACCCAGUACGGAACGACA | 29 | UGUCGUUCCGUACUGGG | 317 |
| M15 | | | UACUUGCCCU | |
| S30-AS318- | AGUACCCAGUACGGAACGACAUGGA | 30 | UCCAUGUCGUUCCGUAC | 318 |
| M15 | | | UGGGUACUUG | |
| S31-AS319- | GUACCCAGUACGGAACGACAUGGAG | 31 | CUCCAUGUCGUUCCGUAC | 319 |
| M15 | | | UGGGUACUU | |
| S32-AS320- | CAGUACGGAACGACAUGGAGCUATG | 32 | CAUAGCUCCAUGUCGUUC | 320 |
| M15 | | | CGUACUGGG | |
| S33-AS321- | GUACGGAACGACAUGGAGCUAUGGA | 33 | UCCAUAGCUCCAUGUCGU | 321 |
| M15 | | | UCCGUACUG | |
| S34-AS322- | UACGGAACGACAUGGAGCUAUGGAA | 34 | UUCCAUAGCUCCAUGUCG | 322 |
| M15 | | | UUCCGUACU | |
| S35-AS323- | ACGGAACGACAUGGAGCUAUGGAAG | 35 | CUUCCAUAGCUCCAUGUC | 323 |
| M15 | | | GUUCCGUAC | |
| S36-AS324- | CUGAACCAGCGGUUGCUGAAGCCAG | 36 | CUGGCUUCAGCAACCGCU | 324 |
| M15 | | | GGUUCAGAG | |
| S37-AS325- | CAGCGCUCUAUACGGAUGCUUUCAA | 37 | UUGAAAGCAUCCGUAUA | 325 |
| M15 | | | GAGCGCUGCU | |
| S38-AS326- | AGCGCUCUAUACGGAUGCUUUCAAT | 38 | AUUGAAAGCAUCCGUAU | 326 |
| M15 | | | AGAGCGCUGC | |
| S39-AS327- | GCGCUCUAUACGGAUGCUUUCAATG | 39 | CAUUGAAAGCAUCCGUAU | 327 |
| M15 | | | AGAGCGCUG | |
| S40-AS328- | CGCUCUAUACGGAUGCUUUCAAUGA | 40 | UCAUUGAAAGCAUCCGUA | 328 |
| M15 | | | UAGAGCGCU | |
| S41-AS329- | GCUCUAUACGGAUGCUUUCAAUGAG | 41 | CUCAUUGAAAGCAUCCGU | 329 |
| M15 | | | AUAGAGCGC | |
| S42-AS330- | CUCUAUACGGAUGCUUUCAAUGAGG | 42 | CCUCAUUGAAAGCAUCCG | 330 |
| M15 | | | UAUAGAGCG | |
| S43-AS331- | UCUAUACGGAUGCUUUCAAUGAGGT | 43 | ACCUCAUUGAAAGCAUCC | 331 |
| M15 | | | GUAUAGAGC | |
| S44-AS332- | CUAUACGGAUGCUUUCAAUGAGGTG | 44 | CACCUCAUUGAAAGCAUC | 332 |
| M15 | | | CGUAUAGAG | |
| S45-AS333- | UAUACGGAUGCUUUCAAUGAGGUGA | 45 | UCACCUCAUUGAAAGCAU | 333 |
| M15 | | | CCGUAUAGA | |
| S46-AS334- | AUACGGAUGCUUUCAAUGAGGUGAT | 46 | AUCACCUCAUUGAAAGCA | 334 |
| M15 | | | UCCGUAUAG | |
| S47-AS335- | UACGGAUGCUUUCAAUGAGGUGATT | 47 | AAUCACCUCAUUGAAAGC | 335 |
| M15 | | | AUCCGUAUA | |
| S48-AS336- | ACGGAUGCUUUCAAUGAGGUGAUTG | 48 | CAAUCACCUCAUUGAAAG | 336 |
| M15 | | | CAUCCGUAU | |
| S49-AS337- | CGGAUGCUUUCAAUGAGGUGAUUG | 49 | UCAAUCACCUCAUUGAAA | 337 |
| M15 | A | | GCAUCCGUA | |
| S50-AS338- | GGAUGCUUUCAAUGAGGUGAUUGAT | 50 | AUCAAUCACCUCAUUGAA | 338 |
| M15 | | | AGCAUCCGU | |
| S51-AS339- | GAUGCUUUCAAUGAGGUGAUUGATG | 51 | CAUCAAUCACCUCAUUGA | 339 |
| M15 | | | AAGCAUCCG | |
| S52-AS340- | AUGCUUUCAAUGAGGUGAUUGAUG | 52 | UCAUCAAUCACCUCAUUG | 340 |
| M15 | A | | AAAGCAUCC | |
| S53-AS341- | UGCUUUCAAUGAGGUGAUUGAUGA | 53 | GUCAUCAAUCACCUCAUU | 341 |
| M15 | C | | GAAAGCAUC | |
| S54-AS342- | GCUUUCAAUGAGGUGAUUGAUGACT | 54 | AGUCAUCAAUCACCUCAU | 342 |
| M15 | | | UGAAAGCAU | |
| S55-AS343- | CUUUCAAUGAGGUGAUUGAUGACTT | 55 | AAGUCAUCAAUCACCUCA | 343 |
| M15 | | | UUGAAAGCA | |
| S56-AS344- | UUUCAAUGAGGUGAUUGAUGACUTT | 56 | AAAGUCAUCAAUCACCUC | 344 |
| M15 | | | AUUGAAAGC | |
| S57-AS345- | UUCAAUGAGGUGAUUGAUGACUUTA | 57 | UAAAGUCAUCAAUCACCU | 345 |
| M15 | | | CAUUGAAAG | |
| S58-AS346- | UCAAUGAGGUGAUUGAUGACUUUAT | 58 | AUAAAGUCAUCAAUCACC | 346 |
| M15 | | | UCAUUGAAA | |
| S59-AS347- | CAAUGAGGUGAUUGAUGACUUUATG | 59 | CAUAAAGUCAUCAAUCAC | 347 |
| M15 | | | CUCAUUGAA | |
| S60-AS348- | AAUGAGGUGAUUGAUGACUUUAUG | 60 | UCAUAAAGUCAUCAAUCA | 348 |
| M15 | A | | CCUCAUUGA | |
| S61-AS349- | AUGAGGUGAUUGAUGACUUUAUGA | 61 | GUCAUAAAGUCAUCAAUC | 349 |
| M15 | C | | ACCUCAUUG | |
| S62-AS350- | UGAGGUGAUUGAUGACUUUAUGACT | 62 | AGUCAUAAAGUCAUCAAU | 350 |
| M15 | | | CACCUCAUU | |
| S63-AS351- | GAGGUGAUUGAUGACUUUAUGACTC | 63 | GAGUCAUAAAGUCAUCAA | 351 |
| M15 | | | UCACCUCAU | |
| S64-AS352- | AGGUGAUUGAUGACUUUAUGACUC | 64 | CGAGUCAUAAAGUCAUCA | 352 |
| M15 | G | | AUCACCUCA | |
| S65-AS353- | GGUGAUUGAUGACUUUAUGACUCG | 65 | UCGAGUCAUAAAGUCAUC | 353 |
| M15 | A | | AAUCACCUC | |
| S66-AS354- | GUGAUUGAUGACUUUAUGACUCGAC | 66 | GUCGAGUCAUAAAGUCA | 354 |
| M15 | | | UCAAUCACCU | |
| S67-AS355- | UGAUUGAUGACUUUAUGACUCGACT | 67 | AGUCGAGUCAUAAAGUC | 355 |
| M15 | | | AUCAAUCACC | |
| S68-AS356- | GAUUGAUGACUUUAUGACUCGACTG | 68 | CAGUCGAGUCAUAAAGUC | 356 |
| M15 | | | AUCAAUCAC | |
| S69-AS357- | AUUGAUGACUUUAUGACUCGACUGG | 69 | CCAGUCGAGUCAUAAAGU | 357 |
| M15 | | | CAUCAAUCA | |
| S70-AS358- | UUGAUGACUUUAUGACUCGACUGGA | 70 | UCCAGUCGAGUCAUAAAG | 358 |
| M15 | | | UCAUCAAUC | |
| S71-AS359- | GAUGACUUUAUGACUCGACUGGACC | 71 | GGUCCAGUCGAGUCAUA | 359 |
| M15 | | | AAGUCAUCAA | |
| S72-AS360- | AUGACUUUAUGACUCGACUGGACCA | 72 | UGGUCCAGUCGAGUCAU | |
| M15 | | | AAAGUCAUCA | 360 |
| S73-AS361- | GACUUUAUGACUCGACUGGACCAGC | 73 | GCUGGUCCAGUCGAGUC | |
| M15 | | | AUAAAGUCAU | 361 |
| S74-AS362- | ACUUUAUGACUCGACUGGACCAGCT | 74 | AGCUGGUCCAGUCGAGU | 362 |
| M15 | | | CAUAAAGUCA | |
| S75-AS363- | UCGGACAUGGCUCAACUCUUCUACT | 75 | AGUAGAAGAGUUGAGCC | 363 |
| M15 | | | AUGUCCGACA | |
| S76-AS364- | GGACAUGGCUCAACUCUUCUACUAC | 76 | GUAGUAGAAGAGUUGAG | 364 |
| M15 | | | CCAUGUCCGA | |
| S77-AS365- | GACAUGGCUCAACUCUUCUACUACT | 77 | AGUAGUAGAAGAGUUGA | 365 |
| M15 | | | GCCAUGUCCG | |
| S78-AS366- | CUCAACUCUUCUACUACUUUGCCTT | 78 | AAGGCAAAGUAGUAGAA | 366 |
| M15 | | | GAGUUGAGCC | |
| S79-AS367- | AACUCUUCUACUACUUUGCCUUGGA | 79 | UCCAAGGCAAAGUAGUA | 367 |
| M15 | | | GAAGAGUUGA | |
| S80-AS368- | ACUCUUCUACUACUUUGCCUUGGAA | 80 | UUCCAAGGCAAAGUAGU | 368 |
| M15 | | | AGAAGAGUUG | |
| S81-AS369- | CUCUUCUACUACUUUGCCUUGGAAG | 81 | CUUCCAAGGCAAAGUAGU | 369 |
| M15 | | | AGAAGAGUU | |
| S82-AS370- | UUCUACUACUUUGCCUUGGAAGCTA | 82 | UAGCUUCCAAGGCAAAGU | 370 |
| M15 | | | AGUAGAAGA | |
| S83-AS371- | UCUACUACUUUGCCUUGGAAGCUAT | 83 | AUAGCUUCCAAGGCAAAG | 371 |
| M15 | | | UAGUAGAAG | |
| S84-AS372- | CUACUACUUUGCCUUGGAAGCUATT | 84 | AAUAGCUUCCAAGGCAAA | 372 |
| M15 | | | GUAGUAGAA | |
| S85-AS373- | UACUACUUUGCCUUGGAAGCUAUTT | 85 | AAAUAGCUUCCAAGGCAA | 373 |
| M15 | | | AGUAGUAGA | |
| S86-AS374- | ACUACUUUGCCUUGGAAGCUAUUTG | 86 | CAAAUAGCUUCCAAGGCA | 374 |
| M15 | | | AAGUAGUAG | |
| S87-AS375- | CUACUUUGCCUUGGAAGCUAUUUGC | 87 | GCAAAUAGCUUCCAAGGC | 375 |
| M15 | | | AAAGUAGUA | |
| S88-AS376- | UACUUUGCCUUGGAAGCUAUUUGCT | 88 | AGCAAAUAGCUUCCAAGG | 376 |
| M15 | | | CAAAGUAGU | |
| S89-AS377- | ACUUUGCCUUGGAAGCUAUUUGCTA | 89 | UAGCAAAUAGCUUCCAAG | 377 |
| M15 | | | GCAAAGUAG | |
| S90-AS378- | CUUUGCCUUGGAAGCUAUUUGCUAC | 90 | GUAGCAAAUAGCUUCCAA | 378 |
| M15 | | | GGCAAAGUA | |
| S91-AS379- | UUUGCCUUGGAAGCUAUUUGCUACA | 91 | UGUAGCAAAUAGCUUCCA | 379 |
| M15 | | | AGGCAAAGU | |
| S92-AS380- | UUGCCUUGGAAGCUAUUUGCUACAT | 92 | AUGUAGCAAAUAGCUUCC | 380 |
| M15 | | | AAGGCAAAG | |
| S93-AS381- | UGCCUUGGAAGCUAUUUGCUACATC | 93 | GAUGUAGCAAAUAGCUU | 381 |
| M15 | | | CCAAGGCAAA | |
| S94-AS382- | GCCUUGGAAGCUAUUUGCUACAUCC | 94 | GGAUGUAGCAAAUAGCU | 382 |
| M15 | | | UCCAAGGCAA | |
| S95-AS383- | CCUUGGAAGCUAUUUGCUACAUCCT | 95 | AGGAUGUAGCAAAUAGC | 383 |
| M15 | | | UUCCAAGGCA | |
| S96-AS384- | CUUGGAAGCUAUUUGCUACAUCCTG | 96 | CAGGAUGUAGCAAAUAG | 384 |
| M15 | | | CUUCCAAGGC | |
| S97-AS385- | UUGGAAGCUAUUUGCUACAUCCUGT | 97 | ACAGGAUGUAGCAAAUA | 385 |
| M15 | | | GCUUCCAAGG | |
| S98-AS386- | UGGAAGCUAUUUGCUACAUCCUGTT | 98 | AACAGGAUGUAGCAAAUA | 386 |
| M15 | | | GCUUCCAAG | |
| S99-AS387- | GGAAGCUAUUUGCUACAUCCUGUTC | 99 | GAACAGGAUGUAGCAAA | 387 |
| M15 | | | UAGCUUCCAA | |
| S100-AS388- | GAAGCUAUUUGCUACAUCCUGUUCG | 100 | CGAACAGGAUGUAGCAAA | 388 |
| M15 | | | UAGCUUCCA | |
| S101-AS389- | AAGCUAUUUGCUACAUCCUGUUCGA | 101 | UCGAACAGGAUGUAGCA | 389 |
| M15 | | | AAUAGCUUCC | |
| S102-AS390- | AGCUAUUUGCUACAUCCUGUUCGAG | 102 | CUCGAACAGGAUGUAGCA | 390 |
| M15 | | | AAUAGCUUC | |
| S103-AS391- | GCUAUUUGCUACAUCCUGUUCGAGA | 103 | UCUCGAACAGGAUGUAG | 391 |
| M15 | | | CAAAUAGCUU | |
| S104-AS392- | CUAUUUGCUACAUCCUGUUCGAGAA | 104 | UUCUCGAACAGGAUGUA | 392 |
| M15 | | | GCAAAUAGCU | |
| S105-AS393- | UAUUUGCUACAUCCUGUUCGAGAAA | 105 | UUUCUCGAACAGGAUGU | 393 |
| M15 | | | AGCAAAUAGC | |
| S106-AS394- | AUUUGCUACAUCCUGUUCGAGAAAC | 106 | GUUUCUCGAACAGGAUG | 394 |
| M15 | | | UAGCAAAUAG | |
| S107-AS395- | UUUGCUACAUCCUGUUCGAGAAACG | 107 | CGUUUCUCGAACAGGAU | 395 |
| M15 | | | GUAGCAAAUA | |
| S108-AS396- | UUGCUACAUCCUGUUCGAGAAACGC | 108 | GCGUUUCUCGAACAGGA | 396 |
| M15 | | | UGUAGCAAAU | |
| S109-AS397- | UGCUACAUCCUGUUCGAGAAACGCA | 109 | UGCGUUUCUCGAACAGG | 397 |
| M15 | | | AUGUAGCAAA | |
| S110-AS398- | GCUACAUCCUGUUCGAGAAACGCAT | 110 | AUGCGUUUCUCGAACAG | 398 |
| M15 | | | GAUGUAGCAA | |
| S111-AS399- | CUACAUCCUGUUCGAGAAACGCATT | 111 | AAUGCGUUUCUCGAACA | 399 |
| M15 | | | GGAUGUAGCA | |
| S112-AS400- | GUCAGAUCCAUCGGGUUAAUGUUCC | 112 | GGAACAUUAACCCGAUGG | 400 |
| M15 | | | AUCUGACGA | |
| S113-AS401- | UCAGAUCCAUCGGGUUAAUGUUCCA | 113 | UGGAACAUUAACCCGAUG | 401 |
| M15 | | | GAUCUGACG | |
| S114-AS402- | CAGAUCCAUCGGGUUAAUGUUCCAG | 114 | CUGGAACAUUAACCCGAU | 402 |
| M15 | | | GGAUCUGAC | |
| S115-AS403- | AGAUCCAUCGGGUUAAUGUUCCAGA | 115 | UCUGGAACAUUAACCCGA | 403 |
| M15 | | | UGGAUCUGA | |
| S116-AS404- | GAUCCAUCGGGUUAAUGUUCCAGAA | 116 | UUCUGGAACAUUAACCCG | 404 |
| M15 | | | AUGGAUCUG | |
| S117-AS405- | AUCCAUCGGGUUAAUGUUCCAGAAC | 117 | GUUCUGGAACAUUAACCC | 405 |
| M15 | | | GAUGGAUCU | |
| S118-AS406- | UCCAUCGGGUUAAUGUUCCAGAACT | 118 | AGUUCUGGAACAUUAACC | 406 |
| M15 | | | CGAUGGAUC | |
| S119-AS407- | CCAUCGGGUUAAUGUUCCAGAACTC | 119 | GAGUUCUGGAACAUUAA | 407 |
| M15 | | | CCCGAUGGAU | |
| S120-AS408- | CAUCGGGUUAAUGUUCCAGAACUCA | 120 | UGAGUUCUGGAACAUUA | 408 |
| M15 | | | ACCCGAUGGA | |
| S121-AS409- | AUCGGGUUAAUGUUCCAGAACUCAC | 121 | GUGAGUUCUGGAACAUU | 409 |
| M15 | | | AACCCGAUGG | |
| S122-AS410- | UCGGGUUAAUGUUCCAGAACUCACT | 122 | AGUGAGUUCUGGAACAU | 410 |
| M15 | | | UAACCCGAUG | |
| S123-AS411- | CGGGUUAAUGUUCCAGAACUCACTC | 123 | GAGUGAGUUCUGGAACA | 411 |
| M15 | | | UUAACCCGAU | |
| S124-AS412- | GGGUUAAUGUUCCAGAACUCACUCT | 124 | AGAGUGAGUUCUGGAAC | 412 |
| M15 | | | AUUAACCCGA | |
| S125-AS413- | GGUUAAUGUUCCAGAACUCACUCTA | 125 | UAGAGUGAGUUCUGGAA | 413 |
| M15 | | | CAUUAACCCG | |
| S126-AS414- | GUUAAUGUUCCAGAACUCACUCUAT | 126 | AUAGAGUGAGUUCUGGA | 414 |
| M15 | | | ACAUUAACCC | |
| S127-AS415- | UUAAUGUUCCAGAACUCACUCUATG | 127 | CAUAGAGUGAGUUCUGG | 415 |
| M15 | | | AACAUUAACC | |
| S128-AS416- | UAAUGUUCCAGAACUCACUCUAUGC | 128 | GCAUAGAGUGAGUUCUG | 416 |
| M15 | | | GAACAUUAAC | |
| S129-AS417- | AAUGUUCCAGAACUCACUCUAUGCC | 129 | GGCAUAGAGUGAGUUCU | 417 |
| M15 | | | GGAACAUUAA | |
| S130-AS418- | AUGUUCCAGAACUCACUCUAUGCCA | 130 | UGGCAUAGAGUGAGUUC | 418 |
| M15 | | | UGGAACAUUA | |
| S131-AS419- | UGUUCCAGAACUCACUCUAUGCCAC | 131 | GUGGCAUAGAGUGAGUU | 419 |
| M15 | | | CUGGAACAUU | |
| S132-AS420- | GUUCCAGAACUCACUCUAUGCCACC | 132 | GGUGGCAUAGAGUGAGU | 420 |
| M15 | | | UCUGGAACAU | |
| S133-AS421- | CCAGAACUCACUCUAUGCCACCUTC | 133 | GAAGGUGGCAUAGAGUG | 421 |
| M15 | | | AGUUCUGGAA | |
| S134-AS422- | CAGAACUCACUCUAUGCCACCUUCC | 134 | GGAAGGUGGCAUAGAGU | 422 |
| M15 | | | GAGUUCUGGA | |
| S135-AS423- | GGAAGCGAUACCUGGAUGGUUGGAA | 135 | UUCCAACCAUCCAGGUAU | 423 |
| M15 | | | CGCUUCCAG | |
| S136-AS424- | GAAGCGAUACCUGGAUGGUUGGAAT | 136 | AUUCCAACCAUCCAGGUA | 424 |
| M15 | | | UCGCUUCCA | |
| S137-AS425- | AAGCGAUACCUGGAUGGUUGGAATG | 137 | CAUUCCAACCAUCCAGGU | 425 |
| M15 | | | AUCGCUUCC | |
| S138-AS426- | AGCGAUACCUGGAUGGUUGGAAUGC | 138 | GCAUUCCAACCAUCCAGG | 426 |
| M15 | | | UAUCGCUUC | |
| S139-AS427- | GCGAUACCUGGAUGGUUGGAAUGCC | 139 | GGCAUUCCAACCAUCCAG | 427 |
| M15 | | | GUAUCGCUU | |
| S140-AS428- | CGAUACCUGGAUGGUUGGAAUGCCA | 140 | UGGCAUUCCAACCAUCCA | 428 |
| M15 | | | GGUAUCGCU | |
| S141-AS429- | GAUACCUGGAUGGUUGGAAUGCCAT | 141 | AUGGCAUUCCAACCAUCC | 429 |
| M15 | | | AGGUAUCGC | |
| S142-AS430- | AUACCUGGAUGGUUGGAAUGCCATC | 142 | GAUGGCAUUCCAACCAUC | 430 |
| M15 | | | CAGGUAUCG | |
| S143-AS431- | ACCUGGAUGGUUGGAAUGCCAUCTT | 143 | AAGAUGGCAUUCCAACCA | 431 |
| M15 | | | UCCAGGUAU | |
| S144-AS432- | CCUGGAUGGUUGGAAUGCCAUCUTT | 144 | AAAGAUGGCAUUCCAACC | 432 |
| M15 | | | AUCCAGGUA | |
| S145-AS433- | CUGGAUGGUUGGAAUGCCAUCUUTT | 145 | AAAAGAUGGCAUUCCAAC | 433 |
| M15 | | | CAUCCAGGU | |
| S146-AS434- | UGGAUGGUUGGAAUGCCAUCUUUTC | 146 | GAAAAGAUGGCAUUCCAA | 434 |
| M15 | | | CCAUCCAGG | |
| S147-AS435- | GGAUGGUUGGAAUGCCAUCUUUUCC | 147 | GGAAAAGAUGGCAUUCC | 435 |
| M15 | | | AACCAUCCAG | |
| S148-AS436- | GAUGGUUGGAAUGCCAUCUUUUCCT | 148 | AGGAAAAGAUGGCAUUC | 436 |
| M15 | | | CAACCAUCCA | |
| S149-AS437- | AUGGUUGGAAUGCCAUCUUUUCCTT | 149 | AAGGAAAAGAUGGCAUU | 437 |
| M15 | | | CCAACCAUCC | |
| S150-AS438- | UGGUUGGAAUGCCAUCUUUUCCUTT | 150 | AAAGGAAAAGAUGGCAU | 438 |
| M15 | | | UCCAACCAUC | |
| S151-AS439- | GGUUGGAAUGCCAUCUUUUCCUUTG | 151 | CAAAGGAAAAGAUGGCAU | 439 |
| M15 | | | UCCAACCAU | |
| S152-AS440- | GUUGGAAUGCCAUCUUUUCCUUUG | 152 | CCAAAGGAAAAGAUGGCA | 440 |
| M15 | G | | UUCCAACCA | |
| S153-AS441- | GGAAUGCCAUCUUUUCCUUUGGGAA | 153 | UUCCCAAAGGAAAAGAUG | 441 |
| M15 | | | GCAUUCCAA | |
| S154-AS442- | CCUUUGGGAAGAAGCUGAUUGAUGA | 154 | UCAUCAAUCAGCUUCUUC | 442 |
| M15 | | | CCAAAGGAA | |
| S155-AS443- | GGGAAGAAGCUGAUUGAUGAGAAGC | 155 | GCUUCUCAUCAAUCAGCU | 443 |
| M15 | | | UCUUCCCAA | |
| S156-AS444- | GGAAGAAGCUGAUUGAUGAGAAGCT | 156 | AGCUUCUCAUCAAUCAGC | 444 |
| M15 | | | UUCUUCCCA | |
| S157-AS445- | GAAGAAGCUGAUUGAUGAGAAGCTC | 157 | GAGCUUCUCAUCAAUCAG | 445 |
| M15 | | | CUUCUUCCC | |
| S158-AS446- | AAGAAGCUGAUUGAUGAGAAGCUCG | 158 | CGAGCUUCUCAUCAAUCA | 446 |
| M15 | | | GCUUCUUCC | |
| S159-AS447- | AGAAGCUGAUUGAUGAGAAGCUCGA | 159 | UCGAGCUUCUCAUCAAUC | 447 |
| M15 | | | AGCUUCUUC | |
| S160-AS448- | GAAGCUGAUUGAUGAGAAGCUCGAA | 160 | UUCGAGCUUCUCAUCAA | 448 |
| M15 | | | UCAGCUUCUU | |
| S161-AS449- | AAGCUGAUUGAUGAGAAGCUCGAAG | 161 | CUUCGAGCUUCUCAUCAA | 449 |
| M15 | | | UCAGCUUCU | |
| S162-AS450- | AGCUGAUUGAUGAGAAGCUCGAAGA | 162 | UCUUCGAGCUUCUCAUCA | 450 |
| M15 | | | AUCAGCUUC | |
| S163-AS451- | GCUGAUUGAUGAGAAGCUCGAAGAT | 163 | AUCUUCGAGCUUCUCAUC | 451 |
| M15 | | | AAUCAGCUU | |
| S164-AS452- | CUGAUUGAUGAGAAGCUCGAAGATA | 164 | UAUCUUCGAGCUUCUCA | 452 |
| M15 | | | UCAAUCAGCU | |
| S165-AS453- | UGAUUGAUGAGAAGCUCGAAGAUAT | 165 | AUAUCUUCGAGCUUCUC | 453 |
| M15 | | | AUCAAUCAGC | |
| S166-AS454- | GAUUGAUGAGAAGCUCGAAGAUATG | 166 | CAUAUCUUCGAGCUUCUC | 454 |
| M15 | | | AUCAAUCAG | |
| S167-AS455- | AUUGAUGAGAAGCUCGAAGAUAUGG | 167 | CCAUAUCUUCGAGCUUCU | 455 |
| M15 | | | CAUCAAUCA | |
| S168-AS456- | UUGAUGAGAAGCUCGAAGAUAUGGA | 168 | UCCAUAUCUUCGAGCUUC | 456 |
| M15 | | | UCAUCAAUC | |
| S169-AS457- | UGAUGAGAAGCUCGAAGAUAUGGAG | 169 | CUCCAUAUCUUCGAGCUU | 457 |
| M15 | | | CUCAUCAAU | |
| S170-AS458- | GAUGAGAAGCUCGAAGAUAUGGAGG | 170 | CCUCCAUAUCUUCGAGCU | 458 |
| M15 | | | UCUCAUCAA | |
| S171-AS459- | CUGACAUGGGCCCUGUACCACCUCT | 171 | AGAGGUGGUACAGGGCC | 459 |
| M15 | | | CAUGUCAGCG | |
| S172-AS460- | UGACAUGGGCCCUGUACCACCUCTC | 172 | GAGAGGUGGUACAGGGC | 460 |
| M15 | | | CCAUGUCAGC | |
| S173-AS461- | GACAUGGGCCCUGUACCACCUCUCA | 173 | UGAGAGGUGGUACAGGG | 461 |
| M15 | | | CCCAUGUCAG | |
| S174-AS462- | ACAUGGGCCCUGUACCACCUCUCAA | 174 | UUGAGAGGUGGUACAGG | 462 |
| M15 | | | GCCCAUGUCA | |
| S175-AS463- | CAUGGGCCCUGUACCACCUCUCAAA | 175 | UUUGAGAGGUGGUACAG | 463 |
| M15 | | | GGCCCAUGUC | |
| S176-AS464- | GAGAUCCAGGAGGCCUUGCACGAGG | 176 | CCUCGUGCAAGGCCUCCU | 464 |
| M15 | | | GGAUCUCAG | |
| S177-AS465- | AGAUCCAGGAGGCCUUGCACGAGGA | 177 | UCCUCGUGCAAGGCCUCC | 465 |
| M15 | | | UGGAUCUCA | |
| S178-AS466- | GAUCCAGGAGGCCUUGCACGAGGAA | 178 | UUCCUCGUGCAAGGCCUC | 466 |
| M15 | | | CUGGAUCUC | |
| S179-AS467- | GUGCCCCAGCACAAGGACUUUGCCC | 179 | GGGCAAAGUCCUUGUGC | 467 |
| M15 | | | UGGGGCACUU | |
| S180-AS468- | UGCCCCAGCACAAGGACUUUGCCCA | 180 | UGGGCAAAGUCCUUGUG | 468 |
| M15 | | | CUGGGGCACU | |
| S181-AS469- | GCCCCAGCACAAGGACUUUGCCCAC | 181 | GUGGGCAAAGUCCUUGU | 469 |
| M15 | | | GCUGGGGCAC | |
| S182-AS470- | CCCCAGCACAAGGACUUUGCCCACA | 182 | UGUGGGCAAAGUCCUUG | 470 |
| M15 | | | UGCUGGGGCA | |
| S183-AS471- | CCCAGCACAAGGACUUUGCCCACAT | 183 | AUGUGGGCAAAGUCCUU | 471 |
| M15 | | | GUGCUGGGGC | |
| S184-AS472- | CCAGCACAAGGACUUUGCCCACATG | 184 | CAUGUGGGCAAAGUCCU | 472 |
| M15 | | | UGUGCUGGGG | |
| S185-AS473- | CAGCACAAGGACUUUGCCCACAUGC | 185 | GCAUGUGGGCAAAGUCC | 473 |
| M15 | | | UUGUGCUGGG | |
| S186-AS474- | AGCACAAGGACUUUGCCCACAUGCC | 186 | GGCAUGUGGGCAAAGUC | 474 |
| M15 | | | CUUGUGCUGG | |
| S187-AS475- | GCACAAGGACUUUGCCCACAUGCCG | 187 | CGGCAUGUGGGCAAAGU | 475 |
| M15 | | | CCUUGUGCUG | |
| S188-AS476- | CACAAGGACUUUGCCCACAUGCCGT | 188 | ACGGCAUGUGGGCAAAG | 476 |
| M15 | | | UCCUUGUGCU | |
| S189-AS477- | ACAAGGACUUUGCCCACAUGCCGTT | 189 | AACGGCAUGUGGGCAAA | 477 |
| M15 | | | GUCCUUGUGC | |
| S190-AS478- | CAAGGACUUUGCCCACAUGCCGUTG | 190 | CAACGGCAUGUGGGCAAA | 478 |
| M15 | | | GUCCUUGUG | |
| S191-AS479- | AAGGACUUUGCCCACAUGCCGUUGC | 191 | GCAACGGCAUGUGGGCAA | 479 |
| M15 | | | AGUCCUUGU | |
| S192-AS480- | AGGACUUUGCCCACAUGCCGUUGCT | 192 | AGCAACGGCAUGUGGGCA | 480 |
| M15 | | | AAGUCCUUG | |
| S193-AS481- | CUCAAAGCUGUGCUUAAGGAGACTC | 193 | GAGUCUCCUUAAGCACAG | 481 |
| M15 | | | CUUUGAGCA | |
| S194-AS482- | CAAAGCUGUGCUUAAGGAGACUCTG | 194 | CAGAGUCUCCUUAAGCAC | 482 |
| M15 | | | AGCUUUGAG | |
| S195-AS483- | CCCACAAACUCCCGGAUCAUAGAAA | 195 | UUUCUAUGAUCCGGGAG | 483 |
| M15 | | | UUUGUGGGGA | |
| S196-AS484- | CCACAAACUCCCGGAUCAUAGAAAA | 196 | UUUUCUAUGAUCCGGGA | 484 |
| M15 | | | GUUUGUGGGG | |
| S197-AS485- | ACAAACUCCCGGAUCAUAGAAAAGG | 197 | CCUUUUCUAUGAUCCGG | 485 |
| M15 | | | GAGUUUGUGG | |
| S198-AS486- | CAAACUCCCGGAUCAUAGAAAAGGA | 198 | UCCUUUUCUAUGAUCCG | 486 |
| M15 | | | GGAGUUUGUG | |
| S199-AS487- | UCCCGGAUCAUAGAAAAGGAAAUTG | 199 | CAAUUUCCUUUUCUAUG | 487 |
| M15 | | | AUCCGGGAGU | |
| S200-AS488- | CCCGGAUCAUAGAAAAGGAAAUUGA | 200 | UCAAUUUCCUUUUCUAU | 488 |
| M15 | | | GAUCCGGGAG | |
| S201-AS489- | CCGGAUCAUAGAAAAGGAAAUUGAA | 201 | UUCAAUUUCCUUUUCUA | 489 |
| M15 | | | UGAUCCGGGA | |
| S202-AS490- | CGGAUCAUAGAAAAGGAAAUUGAAG | 202 | CUUCAAUUUCCUUUUCU | 490 |
| M15 | | | AUGAUCCGGG | |
| S203-AS491- | GGAUCAUAGAAAAGGAAAUUGAAGT | 203 | ACUUCAAUUUCCUUUUC | 491 |
| M15 | | | UAUGAUCCGG | |
| S204-AS492- | GAUCAUAGAAAAGGAAAUUGAAGTT | 204 | AACUUCAAUUUCCUUUU | 492 |
| M15 | | | CUAUGAUCCG | |
| S205-AS493- | AUCAUAGAAAAGGAAAUUGAAGUTG | 205 | CAACUUCAAUUUCCUUU | 493 |
| M15 | | | UCUAUGAUCC | |
| S206-AS494- | UCAUAGAAAAGGAAAUUGAAGUUGA | 206 | UCAACUUCAAUUUCCUU | 494 |
| M15 | | | UUCUAUGAUC | |
| S207-AS495- | CAUAGAAAAGGAAAUUGAAGUUGAT | 207 | AUCAACUUCAAUUUCCUU | 495 |
| M15 | | | UUCUAUGAU | |
| S208-AS496- | AUAGAAAAGGAAAUUGAAGUUGATG | 208 | CAUCAACUUCAAUUUCCU | 496 |
| M15 | | | UUUCUAUGA | |
| S209-AS497- | UAGAAAAGGAAAUUGAAGUUGAUG | 209 | CCAUCAACUUCAAUUUCC | 497 |
| M15 | G | | UUUUCUAUG | |
| S210-AS498- | AGAAAAGGAAAUUGAAGUUGAUGGC | 210 | GCCAUCAACUUCAAUUUC | 498 |
| M15 | | | CUUUUCUAU | |
| S211-AS499- | GAAAAGGAAAUUGAAGUUGAUGGCT | 211 | AGCCAUCAACUUCAAUUU | 499 |
| M15 | | | CCUUUUCUA | |
| S212-AS500- | AAAAGGAAAUUGAAGUUGAUGGCTT | 212 | AAGCCAUCAACUUCAAUU | 500 |
| M15 | | | UCCUUUUCU | |
| S213-AS501- | AAAGGAAAUUGAAGUUGAUGGCUTC | 213 | GAAGCCAUCAACUUCAAU | 501 |
| M15 | | | UUCCUUUUC | |
| S214-AS502- | GGAAAUUGAAGUUGAUGGCUUCCTC | 214 | GAGGAAGCCAUCAACUUC | 502 |
| M15 | | | AAUUUCCUU | |
| S215-AS503- | GAAAUUGAAGUUGAUGGCUUCCUCT | 215 | AGAGGAAGCCAUCAACUU | 503 |
| M15 | | | CAAUUUCCU | |
| S216-AS504- | AAAUUGAAGUUGAUGGCUUCCUCTT | 216 | AAGAGGAAGCCAUCAACU | 504 |
| M15 | | | UCAAUUUCC | |
| S217-AS505- | GCAAGGCUGAUCCAGAAGUACAAGG | 217 | CCUUGUACUUCUGGAUC | 505 |
| M15 | | | AGCCUUGCGA | |
| S218-AS506- | CAAGGCUGAUCCAGAAGUACAAGGT | 218 | ACCUUGUACUUCUGGAU | 506 |
| M15 | | | CAGCCUUGCG | |
| S219-AS507- | AAGGCUGAUCCAGAAGUACAAGGTG | 219 | CACCUUGUACUUCUGGA | 507 |
| M15 | | | UCAGCCUUGC | |
| S220-AS508- | AGGCUGAUCCAGAAGUACAAGGUGG | 220 | CCACCUUGUACUUCUGGA | 508 |
| M15 | | | UCAGCCUUG | |
| S221-AS509- | CGCAUUGUCCUGGUUCCCAAUAAGA | 221 | UCUUAUUGGGAACCAGG | 509 |
| M15 | | | ACAAUGCGGG | |
| S222-AS510- | GCAUUGUCCUGGUUCCCAAUAAGAA | 222 | UUCUUAUUGGGAACCAG | 510 |
| M15 | | | GACAAUGCGG | |
| S223-AS511- | CAUUGUCCUGGUUCCCAAUAAGAAA | 223 | UUUCUUAUUGGGAACCA | 511 |
| M15 | | | GGACAAUGCG | |
| S224-AS512- | AUUGUCCUGGUUCCCAAUAAGAAAG | 224 | CUUUCUUAUUGGGAACC | 512 |
| M15 | | | AGGACAAUGC | |
| S225-AS513- | UUGUCCUGGUUCCCAAUAAGAAAGT | 225 | ACUUUCUUAUUGGGAAC | 513 |
| M15 | | | CAGGACAAUG | |
| S226-AS514- | UGUCCUGGUUCCCAAUAAGAAAGTG | 226 | CACUUUCUUAUUGGGAA | 514 |
| M15 | | | CCAGGACAAU | |
| S227-AS515- | GUCCUGGUUCCCAAUAAGAAAGUGG | 227 | CCACUUUCUUAUUGGGA | 515 |
| M15 | | | ACCAGGACAA | |
| S228-AS516- | ACCCUGAGCUUUUGCCACUUCUATC | 228 | GAUAGAAGUGGCAAAAG | 516 |
| M15 | | | CUCAGGGUGU | |
| S229-AS517- | CCCUGAGCUUUUGCCACUUCUAUCA | 229 | UGAUAGAAGUGGCAAAA | 517 |
| M15 | | | GCUCAGGGUG | |
| S230-AS518- | CCUGAGCUUUUGCCACUUCUAUCAT | 230 | AUGAUAGAAGUGGCAAA | 518 |
| M15 | | | AGCUCAGGGU | |
| S231-AS519- | CUGAGCUUUUGCCACUUCUAUCATT | 231 | AAUGAUAGAAGUGGCAA | 519 |
| M15 | | | AAGCUCAGGG | |
| S232-AS520- | UGAGCUUUUGCCACUUCUAUCAUTT | 232 | AAAUGAUAGAAGUGGCA | 520 |
| M15 | | | AAAGCUCAGG | |
| S233-AS521- | GAGCUUUUGCCACUUCUAUCAUUTT | 233 | AAAAUGAUAGAAGUGGC | 521 |
| M15 | | | AAAAGCUCAG | |
| S234-AS522- | AGCUUUUGCCACUUCUAUCAUUUTT | 234 | AAAAAUGAUAGAAGUGG | 522 |
| M15 | | | CAAAAGCUCA | |
| S235-AS523- | GCUUUUGCCACUUCUAUCAUUUUTG | 235 | CAAAAAUGAUAGAAGUG | 523 |
| M15 | | | GCAAAAGCUC | |
| S236-AS524- | CUUUUGCCACUUCUAUCAUUUUUGA | 236 | UCAAAAAUGAUAGAAGU | 524 |
| M15 | | | GGCAAAAGCU | |
| S237-AS525- | UUUUGCCACUUCUAUCAUUUUUGAG | 237 | CUCAAAAAUGAUAGAAG | 525 |
| M15 | | | UGGCAAAAGC | |
| S238-AS526- | UUUGCCACUUCUAUCAUUUUUGAGC | 238 | GCUCAAAAAUGAUAGAAG | 526 |
| M15 | | | UGGCAAAAG | |
| S239-AS527- | UUGCCACUUCUAUCAUUUUUGAGCA | 239 | UGCUCAAAAAUGAUAGA | 527 |
| M15 | | | AGUGGCAAAA | |
| S240-AS528- | UGCCACUUCUAUCAUUUUUGAGCAA | 240 | UUGCUCAAAAAUGAUAG | 528 |
| M15 | | | AAGUGGCAAA | |
| S241-AS529- | GCCACUUCUAUCAUUUUUGAGCAAC | 241 | GUUGCUCAAAAAUGAUA | 529 |
| M15 | | | GAAGUGGCAA | |
| S242-AS530- | CCACUUCUAUCAUUUUUGAGCAACT | 242 | AGUUGCUCAAAAAUGAU | 530 |
| M15 | | | AGAAGUGGCA | |
| S243-AS531- | CACUUCUAUCAUUUUUGAGCAACTC | 243 | GAGUUGCUCAAAAAUGA | 531 |
| M15 | | | UAGAAGUGGC | |
| S244-AS532- | ACUUCUAUCAUUUUUGAGCAACUCC | 244 | GGAGUUGCUCAAAAAUG | 532 |
| M15 | | | AUAGAAGUGG | |
| S245-AS533- | CUUCUAUCAUUUUUGAGCAACUCCC | 245 | GGGAGUUGCUCAAAAAU | 533 |
| M15 | | | GAUAGAAGUG | |
| S246-AS534- | UUCUAUCAUUUUUGAGCAACUCCCT | 246 | AGGGAGUUGCUCAAAAA | 534 |
| M15 | | | UGAUAGAAGU | |
| S247-AS535- | UCUAUCAUUUUUGAGCAACUCCCTC | 247 | GAGGGAGUUGCUCAAAA | 535 |
| M15 | | | AUGAUAGAAG | |
| S248-AS536- | CUAUCAUUUUUGAGCAACUCCCUCT | 248 | AGAGGGAGUUGCUCAAA | 536 |
| M15 | | | AAUGAUAGAA | |
| S249-AS537- | AUCAUUUUUGAGCAACUCCCUCUCA | 249 | UGAGAGGGAGUUGCUCA | 537 |
| M15 | | | AAAAUGAUAG | |
| S250-AS538- | UCAUUUUUGAGCAACUCCCUCUCAG | 250 | CUGAGAGGGAGUUGCUC | 538 |
| M15 | | | AAAAAUGAUA | |
| S251-AS539- | GAGCAACUCCCUCUCAGCUAAAAGG | 251 | CCUUUUAGCUGAGAGGG | 539 |
| M15 | | | AGUUGCUCAA | |
| S252-AS540- | CGCAUUGCUGUCCUUGGGUAGAATA | 252 | UAUUCUACCCAAGGACAG | 540 |
| M15 | | | CAAUGCGAU | |
| S253-AS541- | GCAUUGCUGUCCUUGGGUAGAAUAT | 253 | AUAUUCUACCCAAGGACA | 541 |
| M15 | | | GCAAUGCGA | |
| S254-AS542- | CAUUGCUGUCCUUGGGUAGAAUATA | 254 | UAUAUUCUACCCAAGGAC | 542 |
| M15 | | | AGCAAUGCG | |
| S255-AS543- | AUUGCUGUCCUUGGGUAGAAUAUAA | 255 | UUAUAUUCUACCCAAGGA | 543 |
| M15 | | | CAGCAAUGC | |
| S256-AS544- | UUGCUGUCCUUGGGUAGAAUAUAAA | 256 | UUUAUAUUCUACCCAAG | 544 |
| M15 | | | GACAGCAAUG | |
| S257-AS545- | UGCUGUCCUUGGGUAGAAUAUAAAA | 257 | UUUUAUAUUCUACCCAA | 545 |
| M15 | | | GGACAGCAAU | |
| S258-AS546- | GCUGUCCUUGGGUAGAAUAUAAAAT | 258 | AUUUUAUAUUCUACCCA | 546 |
| M15 | | | AGGACAGCAA | |
| S259-AS547- | CUGUCCUUGGGUAGAAUAUAAAATA | 259 | UAUUUUAUAUUCUACCC | 547 |
| M15 | | | AAGGACAGCA | |
| S260-AS548- | UGUCCUUGGGUAGAAUAUAAAAUAA | 260 | UUAUUUUAUAUUCUACC | 548 |
| M15 | | | CAAGGACAGC | |
| S261-AS549- | GUCCUUGGGUAGAAUAUAAAAUAAA | 261 | UUUAUUUUAUAUUCUAC | 549 |
| M15 | | | CCAAGGACAG | |
| S262-AS550- | UCCUUGGGUAGAAUAUAAAAUAAAG | 262 | CUUUAUUUUAUAUUCUA | 550 |
| M15 | | | CCCAAGGACA | |
| S263-AS551- | CCUUGGGUAGAAUAUAAAAUAAAGG | 263 | CCUUUAUUUUAUAUUCU | 551 |
| M15 | | | ACCCAAGGAC | |
| S264-AS552- | CUUGGGUAGAAUAUAAAAUAAAGGG | 264 | CCCUUUAUUUUAUAUUC | 552 |
| M15 | | | UACCCAAGGA | |
| S265-AS553- | UUGGGUAGAAUAUAAAAUAAAGGGA | 265 | UCCCUUUAUUUUAUAUU | 553 |
| M15 | | | CUACCCAAGG | |
| S266-AS554- | UGGGUAGAAUAUAAAAUAAAGGGAC | 266 | GUCCCUUUAUUUUAUAU | 554 |
| M15 | | | UCUACCCAAG | |
| S267-AS555- | GGGUAGAAUAUAAAAUAAAGGGACT | 267 | AGUCCCUUUAUUUUAUA | 555 |
| M15 | | | UUCUACCCAA | |
| S268-AS556- | GUAGAAUAUAAAAUAAAGGGACUTT | 268 | AAAGUCCCUUUAUUUUA | 556 |
| M15 | | | UAUUCUACCC | |
| S269-AS557- | UAGAAUAUAAAAUAAAGGGACUUTT | 269 | AAAAGUCCCUUUAUUUU | 557 |
| M15 | | | AUAUUCUACC | |
| S270-AS558- | AGAAUAUAAAAUAAAGGGACUUUTA | 270 | UAAAAGUCCCUUUAUUU | 558 |
| M15 | | | UAUAUUCUAC | |
| S271-AS559- | GAAUAUAAAAUAAAGGGACUUUUAT | 271 | AUAAAAGUCCCUUUAUU | 559 |
| M15 | | | UUAUAUUCUA | |
| S272-AS560- | AAUAUAAAAUAAAGGGACUUUUATT | 272 | AAUAAAAGUCCCUUUAU | 560 |
| M15 | | | UUUAUAUUCU | |
| S273-AS561- | AUAUAAAAUAAAGGGACUUUUAUTT | 273 | AAAUAAAAGUCCCUUUAU | 561 |
| M15 | | | UUUAUAUUC | |
| S274-AS562- | UAUAAAAUAAAGGGACUUUUAUUTC | 274 | GAAAUAAAAGUCCCUUUA | 562 |
| M15 | | | UUUUAUAUU | |
| S275-AS563- | AUAAAAUAAAGGGACUUUUAUUUCT | 275 | AGAAAUAAAAGUCCCUUU | 563 |
| M15 | | | AUUUUAUAU | |
| S276-AS564- | UAAAAUAAAGGGACUUUUAUUUCTT | 276 | AAGAAAUAAAAGUCCCUU | 564 |
| M15 | | | UAUUUUAUA | |
| S277-AS565- | AAAAUAAAGGGACUUUUAUUUCUTA | 277 | UAAGAAAUAAAAGUCCCU | 565 |
| M15 | | | UUAUUUUAU | |
| S278-AS566- | AAAUAAAGGGACUUUUAUUUCUUAT | 278 | AUAAGAAAUAAAAGUCCC | 566 |
| M15 | | | UUUAUUUUA | |
| S279-AS567- | AAUAAAGGGACUUUUAUUUCUUATT | 279 | AAUAAGAAAUAAAAGUCC | 567 |
| M15 | | | CUUUAUUUU | |
| S280-AS568- | AUAAAGGGACUUUUAUUUCUUAUTG | 280 | CAAUAAGAAAUAAAAGUC | 568 |
| M15 | | | CCUUUAUUU | |
| S281-AS569- | UAAAGGGACUUUUAUUUCUUAUUG | 281 | CCAAUAAGAAAUAAAAGU | 569 |
| M15 | G | | CCCUUUAUU | |
| S282-AS570- | AAAGGGACUUUUAUUUCUUAUUGG | 282 | UCCAAUAAGAAAUAAAAG | 570 |
| M15 | A | | UCCCUUUAU | |
| S283-AS571- | AAGGGACUUUUAUUUCUUAUUGGA | 283 | UUCCAAUAAGAAAUAAAA | 571 |
| M15 | A | | GUCCCUUUA | |
| S284-AS572- | AGGGACUUUUAUUUCUUAUUGGAA | 284 | UUUCCAAUAAGAAAUAAA | 572 |
| M15 | A | | AGUCCCUUU | |
| S285-AS573- | GGGACUUUUAUUUCUUAUUGGAAA | 285 | UUUUCCAAUAAGAAAUA | 573 |
| M15 | A | | AAAGUCCCUU | |
| S286-AS574- | GGACUUUUAUUUCUUAUUGGAAAA | 286 | UUUUUCCAAUAAGAAAU | 574 |
| M15 | A | | AAAAGUCCCU | |
| S287-AS575- | GACUUUUAUUUCUUAUUGGAAAAA | 287 | UUUUUUCCAAUAAGAAA | 575 |
| M15 | A | | UAAAAGUCCC | |
| S288-AS576- | ACUUUUAUUUCUUAUUGGAAAAAAA | 288 | UUUUUUUCCAAUAAGAA | 576 |
| M15 | | | AUAAAAGUCC | |
| S577-AS579- | UGCUACAUCCUGUUCGAGAAGCAGC | 577 | UUCUCGAACAGGAUGUA | 579 |
| M1 | CGAAAGGCUGC | | GCAAA | |
| S577-AS579- | UGCUACAUCCUGUUCGAGAAGCAGC | 577 | UUCUCGAACAGGAUGUA | 579 |
| M2 | CGAAAGGCUGC | | GCAAA | |
| S577-AS579- | UGCUACAUCCUGUUCGAGAAGCAGC | 577 | UUCUCGAACAGGAUGUA | 579 |
| M3 | CGAAAGGCUGC | | GCAAA | |
| S577-AS579- | UGCUACAUCCUGUUCGAGAAGCAGC | 577 | UUCUCGAACAGGAUGUA | 579 |
| M4 | CGAAAGGCUGC | | GCAAA | |
| S577-AS579- | UGCUACAUCCUGUUCGAGAAGCAGC | 577 | UUCUCGAACAGGAUGUA | 579 |
| M5 | CGAAAGGCUGC | | GCAAA | |
| S577-AS579- | UGCUACAUCCUGUUCGAGAAGCAGC | 577 | UUCUCGAACAGGAUGUA | 579 |
| M6 | CGAAAGGCUGC | | GCAAA | |
| S577-AS579- | UGCUACAUCCUGUUCGAGAAGCAGC | 577 | UUCUCGAACAGGAUGUA | 579 |
| M7 | CGAAAGGCUGC | | GCAAA | |
| S577-AS579- | UGCUACAUCCUGUUCGAGAAGCAGC | 577 | UUCUCGAACAGGAUGUA | 579 |
| M8 | CGAAAGGCUGC | | GCAAA | |
| S577-AS579- | UGCUACAUCCUGUUCGAGAAGCAGC | 577 | UUCUCGAACAGGAUGUA | 579 |
| M9 | CGAAAGGCUGC | | GCAAA | |
| S577-AS579- | UGCUACAUCCUGUUCGAGAAGCAGC | 577 | UUCUCGAACAGGAUGUA | 579 |
| M10 | CGAAAGGCUGC | | GCAAA | |
| S577-AS579- | UGCUACAUCCUGUUCGAGAAGCAGC | 577 | UUCUCGAACAGGAUGUA | 579 |
| M11 | CGAAAGGCUGC | | GCAAA | |
| S785-AS786- | UGCUACAUCCUGUUCGAGA_GCAGC | 785 | UCUCGAACAGGAUGUAG | 786 |
| M26 | CGAAAGGCUGC | | CAAA | |
| S578-AS580- | CAGAACUCACUCUAUGCCACGCAGCC | 578 | GUGGCAUAGAGUGAGUU | 580 |
| M1 | GAAAGGCUGC | | CUGGA | |
| S578-AS580- | CAGAACUCACUCUAUGCCACGCAGCC | 578 | GUGGCAUAGAGUGAGUU | 580 |
| M2 | GAAAGGCUGC | | CUGGA | |
| S578-AS580- | CAGAACUCACUCUAUGCCACGCAGCC | 578 | GUGGCAUAGAGUGAGUU | 580 |
| M3 | GAAAGGCUGC | | CUGGA | |
| S578-AS580- | CAGAACUCACUCUAUGCCACGCAGCC | 578 | GUGGCAUAGAGUGAGUU | 580 |
| M4 | GAAAGGCUGC | | CUGGA | |
| S578-AS580- | CAGAACUCACUCUAUGCCACGCAGCC | 578 | GUGGCAUAGAGUGAGUU | 580 |
| M5 | GAAAGGCUGC | | CUGGA | |
| S578-AS580- | CAGAACUCACUCUAUGCCACGCAGCC | 578 | GUGGCAUAGAGUGAGUU | 580 |
| M6 | GAAAGGCUGC | | CUGGA | |
| S578-AS580- | CAGAACUCACUCUAUGCCACGCAGCC | 578 | GUGGCAUAGAGUGAGUU | 580 |
| M7 | GAAAGGCUGC | | CUGGA | |
| S578-AS580- | CAGAACUCACUCUAUGCCACGCAGCC | 578 | GUGGCAUAGAGUGAGUU | 580 |
| M8 | GAAAGGCUGC | | CUGGA | |
| S578-AS580- | CAGAACUCACUCUAUGCCACGCAGCC | 578 | GUGGCAUAGAGUGAGUU | 580 |
| M9 | GAAAGGCUGC | | CUGGA | |
| S578-AS580- | CAGAACUCACUCUAUGCCACGCAGCC | 578 | GUGGCAUAGAGUGAGUU | 580 |
| M10 | GAAAGGCUGC | | CUGGA | |
| S578-AS580- | CAGAACUCACUCUAUGCCACGCAGCC | 578 | GUGGCAUAGAGUGAGUU | 580 |
| M11 | GAAAGGCUGC | | CUGGA | |
| S787-AS788- | CAGAACUCACUCUAUGCCA-GCAGCC | 787 | UGGCAUAGAGUGAGUUC | 788 |
| M26 | GAAAGGCUGC | | UGGA | |
| S577-AS579- | UGCUACAUCCUGUUCGAGAAGCAGC | 577 | UUCUCGAACAGGAUGUA | 579 |
| M1 | CGAAAGGCUGC | | GCAAA | |
| S577-AS579- | UGCUACAUCCUGUUCGAGAAGCAGC | 577 | UUCUCGAACAGGAUGUA | 579 |
| M9 | CGAAAGGCUGC | | GCAAA | |
| S581-AS598- | CGGAUGCUUUCAAUGAGGUAGCAGC | 581 | UACCUCAUUGAAAGCAUC | 598 |
| M13 | CGAAAGGCUGC | | CGGG | |
| S581-AS598- | CGGAUGCUUUCAAUGAGGUAGCAGC | 581 | UACCUCAUUGAAAGCAUC | 598 |
| M14 | CGAAAGGCUGC | | CGGG | |
| S582-AS599- | AUGAGGUGAUUGAUGACUUUGCAG | 582 | AAAGUCAUCAAUCACCUC | 599 |
| M13 | CCGAAAGGCUGC | | AUGG | |
| S582-AS599- | AUGAGGUGAUUGAUGACUUUGCAG | 582 | AAAGUCAUCAAUCACCUC | 599 |
| M14 | CCGAAAGGCUGC | | AUGG | |
| S583-AS600- | AGGUGAUUGAUGACUUUAUGGCAG | 583 | CAUAAAGUCAUCAAUCAC | 600 |
| M14 | CCGAAAGGCUGC | | CUGG | |
| S583-AS600- | AGGUGAUUGAUGACUUUAUAGCAGC | 583 | UAUAAAGUCAUCAAUCAC | 600 |
| M14* | CGAAAGGCUGC | | CUGG | |
| S584-AS601- | GUGAUUGAUGACUUUAUGAAGCAGC | 584 | UUCAUAAAGUCAUCAAUC | 601 |
| M13 | CGAAAGGCUGC | | ACGG | |
| S584-AS601- | GUGAUUGAUGACUUUAUGAAGCAGC | 584 | UUCAUAAAGUCAUCAAUC | 601 |
| M14 | CGAAAGGCUGC | | ACGG | |
| S585-AS602- | AUUGAUGACUUUAUGACUCAGCAGC | 585 | UGAGUCAUAAAGUCAUC | 602 |
| M14 | CGAAAGGCUGC | | AAUGG | |
| S586-AS603- | UCUACUACUUUGCCUUGGAAGCAGC | 586 | UUCCAAGGCAAAGUAGU | 603 |
| M14 | CGAAAGGCUGC | | AGAGG | |
| S587-AS604- | CUACUUUGCCUUGGAAGCUAGCAGC | 587 | UAGCUUCCAAGGCAAAGU | 604 |
| M13 | CGAAAGGCUGC | | AGGG | |
| S587-AS604- | CUACUUUGCCUUGGAAGCUAGCAGC | 587 | UAGCUUCCAAGGCAAAGU | 604 |
| M14 | CGAAAGGCUGC | | AGGG | |
| S588-AS605- | AUUUGCUACAUCCUGUUCGAGCAGC | 588 | UCGAACAGGAUGUAGCA | 605 |
| M13 | CGAAAGGCUGC | | AAUGG | |
| S588-AS605- | AUUUGCUACAUCCUGUUCGAGCAGC | 588 | UCGAACAGGAUGUAGCA | 605 |
| M14 | CGAAAGGCUGC | | AAUGG | |
| S589-AS606- | UUGCUACAUCCUGUUCGAGAGCAGC | 589 | UCUCGAACAGGAUGUAG | 606 |
| M13 | CGAAAGGCUGC | | CAAGG | |
| S590-AS607- | UGUUCCAGAACUCACUCUAUGCAGC | 590 | AUAGAGUGAGUUCUGGA | 607 |
| M13 | CGAAAGGCUGC | | ACAGG | |
| S590-AS607- | UGUUCCAGAACUCACUCUAUGCAGC | 590 | AUAGAGUGAGUUCUGGA | 607 |
| M14 | CGAAAGGCUGC | | ACAGG | |
| S591-AS608- | AGAAGCUGAUUGAUGAGAAGGCAGC | 591 | CUUCUCAUCAAUCAGCUU | 608 |
| M13 | CGAAAGGCUGC | | CUGG | |
| S591-AS608- | AGAAGCUGAUUGAUGAGAAAGCAGC | 591 | UUUCUCAUCAAUCAGCU | 608 |
| M13* | CGAAAGGCUGC | | UCUGG | |
| S592-AS609- | AGGACUUUGCCCACAUGCCAGCAGCC | 592 | UGGCAUGUGGGCAAAGU | 609 |
| M14 | GAAAGGCUGC | | CCUGG | |
| S593-AS610- | UCCCGGAUCAUAGAAAAGGAGCAGC | 593 | UCCUUUUCUAUGAUCCG | 610 |
| M13 | CGAAAGGCUGC | | GGAGG | |
| S593-AS610- | UCCCGGAUCAUAGAAAAGGAGCAGC | 593 | UCCUUUUCUAUGAUCCG | 610 |
| M14 | CGAAAGGCUGC | | GGAGG | |
| S594-AS611- | CGGAUCAUAGAAAAGGAAAUGCAGC | 594 | AUUUCCUUUUCUAUGAU | 611 |
| M13 | CGAAAGGCUGC | | CCGGG | |
| S594-AS611- | CGGAUCAUAGAAAAGGAAAUGCAGC | 594 | AUUUCCUUUUCUAUGAU | 611 |
| M14 | CGAAAGGCUGC | | CCGGG | |
| S595-AS612- | GAAAUUGAAGUUGAUGGCUUGCAGC | 595 | AAGCCAUCAACUUCAAUU | 612 |
| M13 | CGAAAGGCUGC | | UCGG | |
| S595-AS612- | GAAAUUGAAGUUGAUGGCUUGCAGC | 595 | AAGCCAUCAACUUCAAUU | 612 |
| M14 | CGAAAGGCUGC | | UCGG | |
| S596-AS613- | AAGGCUGAUCCAGAAGUACAGCAGC | 596 | UGUACUUCUGGAUCAGC | 613 |
| M13 | CGAAAGGCUGC | | CUUGG | |
| S596-AS613- | AAGGCUGAUCCAGAAGUACAGCAGC | 596 | UGUACUUCUGGAUCAGC | 613 |
| M14 | CGAAAGGCUGC | | CUUGG | |
| S597-AS614- | GUCCUUGGGUAGAAUAUAAAGCAGC | 597 | UUUAUAUUCUACCCAAG | 614 |
| M13 | CGAAAGGCUGC | | GACGG | |
| S597-AS614- | GUCCUUGGGUAGAAUAUAAAGCAGC | 597 | UUUAUAUUCUACCCAAG | 614 |
| M14 | CGAAAGGCUGC | | GACGG | |
| S789-AS790- | CGGAACGCUACAAUUUUUAUUCCAG | 789 | CUGGAAUAAAAAUUGUA | 790 |
| M27 | | | GCGUUCCGGU | |
| S759-AS763- | CGGAACGCUACAAUUUUUAUGCAGC | 759 | AUAAAAAUUGUAGCGUU | 763 |
| M16G | CGAAAGGCUGC | | CCGGU | |
| S759-AS763- | CGGAACGCUACAAUUUUUAUGCAGC | 759 | AUAAAAAUUGUAGCGUU | 763 |
| M17G | CGAAAGGCUGC | | CCGGU | |
| S759-AS763- | CGGAACGCUACAAUUUUUAUGCAGC | 759 | AUAAAAAUUGUAGCGUU | 763 |
| M18G | CGAAAGGCUGC | | CCGGU | |
| S760-AS764- | ACAAUUUUUAUUCCAGCUAUGCAGC | 760 | AUAGCUGGAAUAAAAAU | 764 |
| M16G | CGAAAGGCUGC | | UGUAG | |
| S760-AS764- | ACAAUUUUUAUUCCAGCUAUGCAGC | 760 | AUAGCUGGAAUAAAAAU | 764 |
| M19G | CGAAAGGCUGC | | UGUAG | |
| S760-AS764- | ACAAUUUUUAUUCCAGCUAUGCAGC | 760 | AUAGCUGGAAUAAAAAU | 764 |
| M18G | CGAAAGGCUGC | | UGUAG | |
| S760-AS764- | ACAAUUUUUAUUCCAGCUAUGCAGC | 760 | AUAGCUGGAAUAAAAAU | 764 |
| M2OG | CGAAAGGCUGC | | UGUAG | |
| S761-AS765- | ACGAGGUUAUCAGUGACUUUGCAGC | 761 | AAAGUCACUGAUAACCUC | 765 |
| M17G | CGAAAGGCUGC | | GUUU | |
| S761-AS765- | ACGAGGUUAUCAGUGACUUUGCAGC | 761 | AAAGUCACUGAUAACCUC | 765 |
| M18G | CGAAAGGCUGC | | GUUU | |
| S762-AS766- | AGAUCCAGGAGGCCUUGCACGCAGC | 762 | GUGCAAGGCCUCCUGGA | 766 |
| M19G | CGAAAGGCUGC | | UCUCA | |
| S577-AS791- | UGCUACAUCCUGUUCGAGAAGCAGC | 577 | UUCUCGAACAGGAUGUA | 791 |
| M21G | CGAAAGGCUGC | | GCAGG | |
| S581-AS598- | CGGAUGCUUUCAAUGAGGUAGCAGC | 581 | UACCUCAUUGAAAGCAUC | 598 |
| M22G | CGAAAGGCUGC | | CGGG | |
| S582-AS599- | AUGAGGUGAUUGAUGACUUUGCAG | 582 | AAAGUCAUCAAUCACCUC | 599 |
| M22G | CCGAAAGGCUGC | | AUGG | |
| S584-AS601- | GUGAUUGAUGACUUUAUGAAGCAGC | 584 | UUCAUAAAGUCAUCAAUC | 601 |
| M22G | CGAAAGGCUGC | | ACGG | |
| S586-AS603- | UCUACUACUUUGCCUUGGAAGCAGC | 586 | UUCCAAGGCAAAGUAGU | 603 |
| M23G | CGAAAGGCUGC | | AGAGG | |
| S588-AS605- | AUUUGCUACAUCCUGUUCGAGCAGC | 588 | UCGAACAGGAUGUAGCA | 605 |
| M23G | CGAAAGGCUGC | | AAUGG | |
| S590-AS607- | UGUUCCAGAACUCACUCUAUGCAGC | 590 | AUAGAGUGAGUUCUGGA | 607 |
| M23G | CGAAAGGCUGC | | ACAGG | |
| S591-AS608- | AGAAGCUGAUUGAUGAGAAGGCAGC | 591 | CUUCUCAUCAAUCAGCUU | 608 |
| M24G | CGAAAGGCUGC | | CUGG | |
| S591-AS608- | AGAAGCUGAUUGAUGAGAAAGCAGC | 591 | UUUCUCAUCAAUCAGCU | 608 |
| M22G* | CGAAAGGCUGC | | UCUGG | |
| S593-AS610- | UCCCGGAUCAUAGAAAAGGAGCAGC | 593 | UCCUUUUCUAUGAUCCG | 610 |
| M22G | CGAAAGGCUGC | | GGAGG | |
| S594-AS611- | CGGAUCAUAGAAAAGGAAAUGCAGC | 594 | AUUUCCUUUUCUAUGAU | 611 |
| M23G | CGAAAGGCUGC | | CCGGG | |
| S595-AS612- | GAAAUUGAAGUUGAUGGCUUGCAGC | 595 | AAGCCAUCAACUUCAAUU | 612 |
| M23G | CGAAAGGCUGC | | UCGG | |
| S597-AS614- | GUCCUUGGGUAGAAUAUAAAGCAGC | 597 | UUUAUAUUCUACCCAAG | 614 |
| M23G | CGAAAGGCUGC | | GACGG | |
| S760-AS792- | ACAAUUUUUAUUCCAGCUAUGCAGC | 760 | AUAGCUGGAAUAAAAAU | 792 |
| M25G | CGAAAGGCUGC | | UGUGG | |
| S615-AS687- | CCGGAACGCUACAAUUUUUAUUCCA | 615 | UGGAAUAAAAAUUGUAG | 687 |
| M15 | | | CGUUCCGGUC | |
| S616-AS688- | CGGAACGCUACAAUUUUUAUUCCAG | 616 | CUGGAAUAAAAAUUGUA | 688 |
| M15 | | | GCGUUCCGGU | |
| S617-AS689- | ACGCUACAAUUUUUAUUCCAGCUAT | 617 | AUAGCUGGAAUAAAAAU | 689 |
| M15 | | | UGUAGCGUUC | |
| S618-AS690- | CGCUACAAUUUUUAUUCCAGCUATT | 618 | AAUAGCUGGAAUAAAAA | 690 |
| M15 | | | UUGUAGCGUU | |
| S619-AS691- | ACAAUUUUUAUUCCAGCUAUUUCTA | 619 | UAGAAAUAGCUGGAAUA | 691 |
| M15 | | | AAAAUUGUAG | |
| S620-AS692- | CAAUUUUUAUUCCAGCUAUUUCUAC | 620 | GUAGAAAUAGCUGGAAU | 692 |
| M15 | | | AAAAAUUGUA | |
| S621-AS693- | AAUUUUUAUUCCAGCUAUUUCUACA | 621 | UGUAGAAAUAGCUGGAA | 693 |
| M15 | | | UAAAAAUUGU | |
| S622-AS694- | AUUUUUAUUCCAGCUAUUUCUACAA | 622 | UUGUAGAAAUAGCUGGA | 694 |
| M15 | | | AUAAAAAUUG | |
| S623-AS695- | CAGGUGCUGAACAAGACCAAGUATG | 623 | CAUACUUGGUCUUGUUC | 695 |
| M15 | | | AGCACCUGGA | |
| S624-AS696- | AACGAGGUUAUCAGUGACUUUAUCA | 624 | UGAUAAAGUCACUGAUA | 696 |
| M15 | | | ACCUCGUUUA | |
| S625-AS697- | ACGAGGUUAUCAGUGACUUUAUCAC | 625 | GUGAUAAAGUCACUGAU | 697 |
| M15 | | | AACCUCGUUU | |
| S626-AS698- | CGAGGUUAUCAGUGACUUUAUCACC | 626 | GGUGAUAAAGUCACUGA | 698 |
| M15 | | | UAACCUCGUU | |
| S627-AS699- | GGAAGCCAUCACCUAUAUCCUGUTT | 627 | AAACAGGAUAUAGGUGA | 699 |
| M15 | | | UGGCUUCCAA | |
| S628-AS700- | GAAGCCAUCACCUAUAUCCUGUUTG | 628 | CAAACAGGAUAUAGGUG | 700 |
| M15 | | | AUGGCUUCCA | |
| S629-AS701- | AAGCCAUCACCUAUAUCCUGUUUGA | 629 | UCAAACAGGAUAUAGGU | 701 |
| M15 | | | GAUGGCUUCC | |
| S630-AS702- | GCCAUCACCUAUAUCCUGUUUGAGA | 630 | UCUCAAACAGGAUAUAG | 702 |
| M15 | | | GUGAUGGCUU | |
| S631-AS703- | CCAUCACCUAUAUCCUGUUUGAGAA | 631 | UUCUCAAACAGGAUAUA | 703 |
| M15 | | | GGUGAUGGCU | |
| S632-AS704- | CAUCACCUAUAUCCUGUUUGAGAAA | 632 | UUUCUCAAACAGGAUAU | 704 |
| M15 | | | AGGUGAUGGC | |
| S633-AS705- | AUCACCUAUAUCCUGUUUGAGAAAA | 633 | UUUUCUCAAACAGGAUA | 705 |
| M15 | | | UAGGUGAUGG | |
| S634-AS706- | ACCUAUAUCCUGUUUGAGAAAAGGA | 634 | UCCUUUUCUCAAACAGGA | 706 |
| M15 | | | UAUAGGUGA | |
| S635-AS707- | CCUAUAUCCUGUUUGAGAAAAGGAT | 635 | AUCCUUUUCUCAAACAGG | 707 |
| M15 | | | AUAUAGGUG | |
| S636-AS708- | CUAUAUCCUGUUUGAGAAAAGGATT | 636 | AAUCCUUUUCUCAAACAG | 708 |
| M15 | | | GAUAUAGGU | |
| S637-AS709- | AGAUCUGUUGCAAUCAUGUUCCAGA | 637 | UCUGGAACAUGAUUGCA | 709 |
| M15 | | | ACAGAUCUGA | |
| S638-AS710- | GAUCUGUUGCAAUCAUGUUCCAGAA | 638 | UUCUGGAACAUGAUUGC | 710 |
| M15 | | | AACAGAUCUG | |
| S639-AS711- | UGUUGCAAUCAUGUUCCAGAACUCA | 639 | UGAGUUCUGGAACAUGA | 711 |
| M15 | | | UUGCAACAGA | |
| S640-AS712- | GUUGCAAUCAUGUUCCAGAACUCAG | 640 | CUGAGUUCUGGAACAUG | 712 |
| M15 | | | AUUGCAACAG | |
| S641-AS713- | CAAUCAUGUUCCAGAACUCAGUCTA | 641 | UAGACUGAGUUCUGGAA | 713 |
| M15 | | | CAUGAUUGCA | |
| S642-AS714- | AUCAUGUUCCAGAACUCAGUCUATA | 642 | UAUAGACUGAGUUCUGG | 714 |
| M15 | | | AACAUGAUUG | |
| S643-AS715- | AUGUUCCAGAACUCAGUCUAUAUCA | 643 | UGAUAUAGACUGAGUUC | 715 |
| M15 | | | UGGAACAUGA | |
| S644-AS716- | UGUUCCAGAACUCAGUCUAUAUCAC | 644 | GUGAUAUAGACUGAGUU | 716 |
| M15 | | | CUGGAACAUG | |
| S645-AS717- | GUUCCAGAACUCAGUCUAUAUCACT | 645 | AGUGAUAUAGACUGAGU | 717 |
| M15 | | | UCUGGAACAU | |
| S646-AS718- | UUCCAGAACUCAGUCUAUAUCACTT | 646 | AAGUGAUAUAGACUGAG | 718 |
| M15 | | | UUCUGGAACA | |
| S647-AS719- | CCAGAACUCAGUCUAUAUCACUUTC | 647 | GAAAGUGAUAUAGACUG | 719 |
| M15 | | | AGUUCUGGAA | |
| S648-AS720- | GAACUCAGUCUAUAUCACUUUCCTT | 648 | AAGGAAAGUGAUAUAGA | 720 |
| M15 | | | CUGAGUUCUG | |
| S649-AS721- | AUAACAUUUUCUCCUUUGGAAAGAA | 649 | UUCUUUCCAAAGGAGAA | 721 |
| M15 | | | AAUGUUAUCC | |
| S650-AS722- | UAACAUUUUCUCCUUUGGAAAGAAG | 650 | CUUCUUUCCAAAGGAGAA | 722 |
| M15 | | | AAUGUUAUC | |
| S651-AS723- | AACAUUUUCUCCUUUGGAAAGAAGC | 651 | GCUUCUUUCCAAAGGAG | 723 |
| M15 | | | AAAAUGUUAU | |
| S652-AS724- | GGAAAGAAGCUGAUUGAUGAAAAAG | 652 | CUUUUUCAUCAAUCAGC | 724 |
| M15 | | | UUCUUUCCAA | |
| S653-AS725- | GAAAGAAGCUGAUUGAUGAAAAAGT | 653 | ACUUUUUCAUCAAUCAGC | 725 |
| M15 | | | UUCUUUCCA | |
| S654-AS726- | AGAAGCUGAUUGAUGAAAAAGUCCA | 654 | UGGACUUUUUCAUCAAU | 726 |
| M15 | | | CAGCUUCUUU | |
| S655-AS727- | CUGCUGACCAAUGAAUUGCUCAGTA | 655 | UACUGAGCAAUUCAUUG | 727 |
| M15 | | | GUCAGCAGGA | |
| S656-AS728- | GCUGACCAAUGAAUUGCUCAGUACT | 656 | AGUACUGAGCAAUUCAU | 728 |
| M15 | | | UGGUCAGCAG | |
| S657-AS729- | CUGACCAAUGAAUUGCUCAGUACTC | 657 | GAGUACUGAGCAAUUCA | 729 |
| M15 | | | UUGGUCAGCA | |
| S658-AS730- | GACCAAUGAAUUGCUCAGUACUCAG | 658 | CUGAGUACUGAGCAAUU | 730 |
| M15 | | | CAUUGGUCAG | |
| S659-AS731- | ACCAAUGAAUUGCUCAGUACUCAGG | 659 | CCUGAGUACUGAGCAAU | 731 |
| M15 | | | UCAUUGGUCA | |
| S660-AS732- | CCAAUGAAUUGCUCAGUACUCAGGA | 660 | UCCUGAGUACUGAGCAA | 732 |
| M15 | | | UUCAUUGGUC | |
| S661-AS733- | AAUGAAUUGCUCAGUACUCAGGAGA | 661 | UCUCCUGAGUACUGAGCA | 733 |
| M15 | | | AUUCAUUGG | |
| S662-AS734- | AUGAAUUGCUCAGUACUCAGGAGAC | 662 | GUCUCCUGAGUACUGAG | 734 |
| M15 | | | CAAUUCAUUG | |
| S663-AS735- | GGAUCAUCACAGAAAAGGAAACUGA | 663 | UCAGUUUCCUUUUCUGU | 735 |
| M15 | | | GAUGAUCCGG | |
| S664-AS736- | GAUCAUCACAGAAAAGGAAACUGAA | 664 | UUCAGUUUCCUUUUCUG | 736 |
| M15 | | | UGAUGAUCCG | |
| S665-AS737- | AUCACAGAAAAGGAAACUGAAAUTA | 665 | UAAUUUCAGUUUCCUUU | 737 |
| M15 | | | UCUGUGAUGA | |
| S666-AS738- | UCACAGAAAAGGAAACUGAAAUUAA | 666 | UUAAUUUCAGUUUCCUU | 738 |
| M15 | | | UUCUGUGAUG | |
| S667-AS739- | CACAGAAAAGGAAACUGAAAUUAAT | 667 | AUUAAUUUCAGUUUCCU | 739 |
| M15 | | | UUUCUGUGAU | |
| S668-AS740- | ACAGAAAAGGAAACUGAAAUUAATG | 668 | CAUUAAUUUCAGUUUCC | 740 |
| M15 | | | UUUUCUGUGA | |
| S669-AS741- | GAAAAGGAAACUGAAAUUAAUGGCT | 669 | AGCCAUUAAUUUCAGUU | 741 |
| M15 | | | UCCUUUUCUG | |
| S670-AS742- | AAAAGGAAACUGAAAUUAAUGGCTT | 670 | AAGCCAUUAAUUUCAGU | 742 |
| M15 | | | UUCCUUUUCU | |
| S671-AS743- | GGAAACUGAAAUUAAUGGCUUUCTC | 671 | GAGAAAGCCAUUAAUUU | 743 |
| M15 | | | CAGUUUCCUU | |
| S672-AS744- | AGACAGCAGAGCACCUUAUAAUAAC | 672 | GUUAUUAUAAGGUGCUC | 744 |
| M15 | | | UGCUGUCUUA | |
| S673-AS745- | GACAGCAGAGCACCUUAUAAUAACA | 673 | UGUUAUUAUAAGGUGCU | 745 |
| M15 | | | CUGCUGUCUU | |
| S674-AS746- | CAGCAGAGCACCUUAUAAUAACAGT | 674 | ACUGUUAUUAUAAGGUG | 746 |
| M15 | | | CUCUGCUGUC | |
| S675-AS747- | AGCAGAGCACCUUAUAAUAACAGTC | 675 | GACUGUUAUUAUAAGGU | 747 |
| M15 | | | GCUCUGCUGU | |
| S676-AS748- | GAGCACCUUAUAAUAACAGUCCUTG | 676 | CAAGGACUGUUAUUAUA | 748 |
| M15 | | | AGGUGCUCUG | |
| S677-AS749- | AUAAUAACAGUCCUUGGGUAUGATT | 677 | AAUCAUACCCAAGGACUG | 749 |
| M15 | | | UUAUUAUAA | |
| S678-AS750- | ACAGUCCUUGGGUAUGAUUUAAAAT | 678 | AUUUUAAAUCAUACCCAA | 750 |
| M15 | | | GGACUGUUA | |
| S679-AS751- | CAGUCCUUGGGUAUGAUUUAAAATA | 679 | UAUUUUAAAUCAUACCCA | 751 |
| M15 | | | AGGACUGUU | |
| S680-AS752- | AGUCCUUGGGUAUGAUUUAAAAUAA | 680 | UUAUUUUAAAUCAUACC | 752 |
| M15 | | | CAAGGACUGU | |
| S681-AS753- | GUCCUUGGGUAUGAUUUAAAAUAAA | 681 | UUUAUUUUAAAUCAUAC | 753 |
| M15 | | | CCAAGGACUG | |
| S682-AS754- | UCCUUGGGUAUGAUUUAAAAUAAAA | 682 | UUUUAUUUUAAAUCAUA | 754 |
| M15 | | | CCCAAGGACU | |
| S683-AS755- | CUUGGGUAUGAUUUAAAAUAAAATT | 683 | AAUUUUAUUUUAAAUCA | 755 |
| M15 | | | UACCCAAGGA | |
| S684-AS756- | UUGGGUAUGAUUUAAAAUAAAAUTT | 684 | AAAUUUUAUUUUAAAUC | 756 |
| M15 | | | AUACCCAAGG | |
| S685-AS757- | UGGGUAUGAUUUAAAAUAAAAUUTA | 685 | UAAAUUUUAUUUUAAAU | 757 |
| M15 | | | CAUACCCAAG | |
| S686-AS758- | GGGUAUGAUUUAAAAUAAAAUUUA | 686 | UUAAAUUUUAUUUUAAA | 758 |
| M15 | A | | UCAUACCCAA | |
| S171-AS459- | CUGACAUGGGCCCUGUACCACCUCT | 171 | AGAGGUGGUACAGGGCC | 459 |
| M15 | | | CAUGUCAGCG | |
| S172-AS460- | UGACAUGGGCCCUGUACCACCUCTC | 172 | GAGAGGUGGUACAGGGC | 460 |
| M15 | | | CCAUGUCAGC | |
| S173-AS461- | GACAUGGGCCCUGUACCACCUCUCA | 173 | UGAGAGGUGGUACAGGG | 461 |
| M15 | | | CCCAUGUCAG | |
| S174-AS462- | ACAUGGGCCCUGUACCACCUCUCAA | 174 | UUGAGAGGUGGUACAGG | 462 |
| M15 | | | GCCCAUGUCA | |
| S175-AS463- | CAUGGGCCCUGUACCACCUCUCAAA | 175 | UUUGAGAGGUGGUACAG | 463 |
| M15 | | | GGCCCAUGUC | |
| S176-AS464- | GAGAUCCAGGAGGCCUUGCACGAGG | 176 | CCUCGUGCAAGGCCUCCU | 464 |
| M15 | | | GGAUCUCAG | |
| S177-AS465- | AGAUCCAGGAGGCCUUGCACGAGGA | 177 | UCCUCGUGCAAGGCCUCC | 465 |
| M15 | | | UGGAUCUCA | |
| S178-AS466- | GAUCCAGGAGGCCUUGCACGAGGAA | 178 | UUCCUCGUGCAAGGCCUC | 466 |
| M15 | | | CUGGAUCUC | |
| S179-AS467- | GUGCCCCAGCACAAGGACUUUGCCC | 179 | GGGCAAAGUCCUUGUGC | 467 |
| M15 | | | UGGGGCACUU | |
| S180-AS468- | UGCCCCAGCACAAGGACUUUGCCCA | 180 | UGGGCAAAGUCCUUGUG | 468 |
| M15 | | | CUGGGGCACU | |
| S181-AS469- | GCCCCAGCACAAGGACUUUGCCCAC | 181 | GUGGGCAAAGUCCUUGU | 469 |
| M15 | | | GCUGGGGCAC | |
| S182-AS470- | CCCCAGCACAAGGACUUUGCCCACA | 182 | UGUGGGCAAAGUCCUUG | 470 |
| M15 | | | UGCUGGGGCA | |
| S183-AS471- | CCCAGCACAAGGACUUUGCCCACAT | 183 | AUGUGGGCAAAGUCCUU | 471 |
| M15 | | | GUGCUGGGGC | |
| S184-AS472- | CCAGCACAAGGACUUUGCCCACATG | 184 | CAUGUGGGCAAAGUCCU | 472 |
| M15 | | | UGUGCUGGGG | |
| S185-AS473- | CAGCACAAGGACUUUGCCCACAUGC | 185 | GCAUGUGGGCAAAGUCC | 473 |
| M15 | | | UUGUGCUGGG | |
| S186-AS474- | AGCACAAGGACUUUGCCCACAUGCC | 186 | GGCAUGUGGGCAAAGUC | 474 |
| M15 | | | CUUGUGCUGG | |
| S187-AS475- | GCACAAGGACUUUGCCCACAUGCCG | 187 | CGGCAUGUGGGCAAAGU | 475 |
| M15 | | | CCUUGUGCUG | |
| S188-AS476- | CACAAGGACUUUGCCCACAUGCCGT | 188 | ACGGCAUGUGGGCAAAG | 476 |
| M15 | | | UCCUUGUGCU | |
| S189-AS477- | ACAAGGACUUUGCCCACAUGCCGTT | 189 | AACGGCAUGUGGGCAAA | 477 |
| M15 | | | GUCCUUGUGC | |
| S190-AS478- | CAAGGACUUUGCCCACAUGCCGUTG | 190 | CAACGGCAUGUGGGCAAA | 478 |
| M15 | | | GUCCUUGUG | |
| S191-AS479- | AAGGACUUUGCCCACAUGCCGUUGC | 191 | GCAACGGCAUGUGGGCAA | 479 |
| M15 | | | AGUCCUUGU | |
| S192-AS480- | AGGACUUUGCCCACAUGCCGUUGCT | 192 | AGCAACGGCAUGUGGGCA | 480 |
| M15 | | | AAGUCCUUG | |
| S195-AS483- | CCCACAAACUCCCGGAUCAUAGAAA | 195 | UUUCUAUGAUCCGGGAG | 483 |
| M15 | | | UUUGUGGGGA | |
| S196-AS484- | CCACAAACUCCCGGAUCAUAGAAAA | 196 | UUUUCUAUGAUCCGGGA | 484 |
| M15 | | | GUUUGUGGGG | |

What is claimed is:

1. An oligonucleotide for reducing expression of CYP27A1, the oligonucleotide comprising an antisense strand comprising a sequence as set forth in any one of SEQ ID NOs: 579-580, 598-614, 763-766, 786, and 788.

2. The oligonucleotide of claim 1, further comprising a sense strand comprises a sequence as set forth in any one of SEQ ID NOs: 577-578, 581-597, 759-762, 785, and 787.

3. The oligonucleotide of claim 1 or 2, wherein the antisense strand consists of a sequence as set forth in any one of SEQ ID NOs: 579-580, 598-614, 763-766, 786, and 788.

4. The oligonucleotide of any one of claims 1 to 3, wherein the sense strand consists of a sequence as set forth in any one of SEQ ID NOs: 577-578, 581-597, 759-762, 785, and 787.

5. An oligonucleotide for reducing expression of CYP27A1, the oligonucleotide comprising an antisense strand of 15 to 30 nucleotides in length, wherein the antisense strand has a region of complementarity to CYP27A1 that is complementary to at least 15 contiguous nucleotides of a sequence as set forth in SEQ ID NOs: 767-781.

6. The oligonucleotide of claim 1, wherein the antisense strand is 19 to 27 nucleotides in length.

7. The oligonucleotide of claim 1, wherein the antisense strand is 21 to 27 nucleotides in length.

8. The oligonucleotide of any one of claims 2 to 4, wherein the sense strand is 15 to 50 nucleotides in length, wherein the sense strand forms a duplex region with the antisense strand.

9. The oligonucleotide of claim 5, wherein the sense strand is 19 to 50 nucleotides in length.

10. The oligonucleotide of claim 5 or 6, wherein the duplex region is at least 19 nucleotides in length.

11. The oligonucleotide of any one of claims 1 to 7, wherein the region of complementarity with CYP27A1 is complementary to at least 19 contiguous nucleotides of a sequence as set forth in SEQ ID NOs: 767-781.

12. The oligonucleotide of any one of claims 5 to 9, wherein the sense strand comprises a sequence as set forth in any one of SEQ ID NOs: 577-578, 581-597, 759-762, 785, and 787.

13. The oligonucleotide of any one of claim 10, wherein the antisense strand comprises a sequence as set forth in any one of SEQ ID NOs: 579-580, 598-614, 763-766, 786, and 788.

14. The oligonucleotide of any one of claims 5 to 9, wherein the sense strand consists of a sequence as set forth in any one of SEQ ID NOs: 577-578, 581-597, 759-762, 785, and 787.

15. The oligonucleotide of any one of claim 10, wherein the antisense strand consists of a sequence as set forth in any one of SEQ ID NOs: 579-580, 598-614, 763-766, 786, and 788.

16. The oligonucleotide of any one of claims 8 to 15, wherein the sense strand comprises at its 3′-end a stem-loop set forth as: S₁-L-S₂, wherein S₁ is complementary to S₂, and wherein L forms a loop between S₁ and S₂ of 3 to 5 nucleotides in length.

17. An oligonucleotide for reducing expression of CYP27A1, the oligonucleotide comprising an antisense strand and a sense strand,
wherein the antisense strand is 21 to 27 nucleotides in length and has a region of complementarity with CYP27A1,
wherein the sense strand comprises at its 3′-end a stem-loop set forth as: S₁-L-S₂, wherein S₁ is complementary to S₂, and wherein L forms a loop between S₁ and S₂ of 3 to 5 nucleotides in length,
and wherein the antisense strand and the sense strand form a duplex structure of at least 19 nucleotides in length but are not covalently linked.

18. The oligonucleotide of claim 17, wherein the region of complementarity is complementary to at least 19 contiguous nucleotides of CYP27A1 mRNA.

19. The oligonucleotide of any one of claims 16 to 18, wherein L is a tetraloop.

20. The oligonucleotide of any one of claims 16 to 19, wherein L is 4 nucleotides in length.

21. The oligonucleotide of any one of claims 16 to 20, wherein L comprises a sequence set forth as GAAA.

22. The oligonucleotide of any one of claims 8 to 15, wherein the antisense strand is 27 nucleotides in length and the sense strand is 25 nucleotides in length.

23. The oligonucleotide of claim 22, wherein the antisense strand and sense strand form a duplex region of 25 nucleotides in length.

24. The oligonucleotide of claim 19, further comprising a 3′-overhang sequence on the antisense strand of two nucleotides in length.

25. The oligonucleotide of any one of claims 8 to 15, wherein the oligonucleotide comprises an antisense strand and a sense strand that are each in a range of 21 to 23 nucleotides in length.

26. The oligonucleotide of claim 25, wherein the oligonucleotide comprises a duplex structure in a range of 19 to 21 nucleotides in length.

27. The oligonucleotide of claim 25 or 26, wherein the oligonucleotide comprises a 3′-overhang sequence of one or more nucleotides in length, wherein the 3′-overhang sequence is present on the antisense strand, the sense strand, or the antisense strand and sense strand.

28. The oligonucleotide of claim 25 or 26, wherein the oligonucleotide comprises a 3′-overhang sequence of two nucleotides in length, wherein the 3′-overhang sequence is present on the antisense strand, and wherein the sense strand is 21 nucleotides in length and the antisense strand is 23 nucleotides in length, such that the sense strand and antisense strand form a duplex of 21 nucleotides in length.

29. The oligonucleotide of any one of the preceding claims, wherein the oligonucleotide comprises at least one modified nucleotide.

30. The oligonucleotide of claim 29, wherein the modified nucleotide comprises a 2′-modification.

31. The oligonucleotide of claim 30, wherein the 2′-modification is a modification selected from: 2′-aminoethyl, 2′-fluoro, 2′-O-methyl, 2′-O-methoxyethyl, and 2′-deoxy-2′-fluoro-β-d-arabinonucleic acid.

32. The oligonucleotide of any one of claims 29 to 31, wherein all of the nucleotides of the oligonucleotide are modified.

33. The oligonucleotide of any one of the preceding claims, wherein the oligonucleotide comprises at least one modified internucleotide linkage.

34. The oligonucleotide of claim 33, wherein the at least one modified internucleotide linkage is a phosphorothioate linkage.

35. The oligonucleotide of any one of the preceding claims, wherein the 4′-carbon of the sugar of the 5′-nucleotide of the antisense strand comprises a phosphate analog.

36. The oligonucleotide of claim 35, wherein the phosphate analog is oxymethyl phosphonate, vinyl phosphonate, or malonyl phosphonate.

37. The oligonucleotide of any one of the preceding claims, wherein at least one nucleotide of the oligonucleotide is conjugated to one or more targeting ligands.

38. The oligonucleotide of claim 37, wherein each targeting ligand comprises a carbohydrate, amino sugar, cholesterol, polypeptide or lipid.

39. The oligonucleotide of claim 38, wherein each targeting ligand comprises a N-acetylgalactosamine (GalNAc) moiety.

40. The oligonucleotide of claim 39, wherein the GalNac moiety is a monovalent GalNAc moiety, a bivalent GalNAc moiety, a trivalent GalNAc moiety, or a tetravalent GalNAc moiety.

41. The oligonucleotide of any one of claims 16 to 19, wherein up to 4 nucleotides of L of the stem-loop are each conjugated to a monovalent GalNAc moiety.

42. The oligonucleotide of claim 37, wherein the targeting ligand comprises an aptamer.

43. A composition comprising an oligonucleotide of any one of the preceding claims and an excipient.

44. A method of delivering an oligonucleotide to a subject, the method comprising administering the composition of claim 43 to the subject.

45. A method of attenuating bile acid accumulation in liver of a subject, the method comprising administering the composition of claim 43 to the subject.

46. A method of decreasing the extent of liver fibrosis in a subject in need thereof, the method comprising administering the composition of claim 43 to the subject.

47. A method of decreasing circulating bile acid concentrations in a subject in need thereof, the method comprising administering the composition of claim 43 to the subject.

48. The method of any one of claims 35 to 47, wherein the subject suffers from hepatobiliary disease.

49. An oligonucleotide for reducing expression of CYP27A1, the oligonucleotide comprising a sense strand of 15 to 50 nucleotides in length and an antisense strand of 15 to 30 nucleotides in length, wherein the sense strand forms a duplex region with the antisense strand, wherein the sense strand comprises a sequence as set forth in any one of SEQ ID NOs: 577-578, 581-597, 759-762, 785, and 787 and wherein the antisense strand comprises a complementary sequence selected from SEQ ID NOs: 579-580, 598-614, 763-766, 786, and 788.

50. An oligonucleotide for reducing expression of CYP27A1, the oligonucleotide comprising a pair of sense and antisense strands selected from a row of the table set forth in Appendix A.

51. The method of any one of claims 35 to 47, wherein the subject suffers from PNALD.


SEQUENCE LISTING (792 sequences)

SEQ ID NO: 1 - DNA, 25 bp, Artificial Sequence
  Synthetic Polynucleotide
ccagaguucagaccaagcgaaaagt

SEQ ID NO: 2 - DNA, 25 bp, Artificial Sequence
  Synthetic Polynucleotide
cagaguucagaccaagcgaaaagtt

SEQ ID NO: 3 - DNA, 25 bp, Artificial Sequence
  Synthetic Polynucleotide
agaguucagaccaagcgaaaaguta

SEQ ID NO: 4 - DNA, 25 bp, Artificial Sequence
  Synthetic Polynucleotide
gaguucagaccaagcgaaaaguuat

SEQ ID NO: 5 - DNA, 25 bp, Artificial Sequence
  Synthetic Polynucleotide
aguucagaccaagcgaaaaguuatt

SEQ ID NO: 6 - DNA, 25 bp, Artificial Sequence
  Synthetic Polynucleotide
guucagaccaagcgaaaaguuautt

SEQ ID NO: 7 - DNA, 25 bp, Artificial Sequence
  Synthetic Polynucleotide
uucagaccaagcgaaaaguuauutg

SEQ ID NO: 8 - RNA, 25 nt, Artificial Sequence
  Synthetic Polynucleotide
ucagaccaagcgaaaaguuauuuga

SEQ ID NO: 9 - RNA, 25 nt, Artificial Sequence
  Synthetic Polynucleotide
cagaccaagcgaaaaguuauuugag

SEQ ID NO: 10 - RNA, 25 nt, Artificial Sequence
  Synthetic Polynucleotide
agaccaagcgaaaaguuauuugaga

SEQ ID NO: 11 - RNA, 25 nt, Artificial Sequence
  Synthetic Polynucleotide
gaccaagcgaaaaguuauuugagag

SEQ ID NO: 12 - RNA, 25 nt, Artificial Sequence
  Synthetic Polynucleotide
accaagcgaaaaguuauuugagagg

SEQ ID NO: 13 - RNA, 25 nt, Artificial Sequence
  Synthetic Polynucleotide
ccaagcgaaaaguuauuugagaggc

SEQ ID NO: 14 - RNA, 25 nt, Artificial Sequence
  Synthetic Polynucleotide
cugcaccaguuacaggugcuuuaca

SEQ ID NO: 15 - RNA, 25 nt, Artificial Sequence
  Synthetic Polynucleotide
ugcaccaguuacaggugcuuuacaa

SEQ ID NO: 16 - RNA, 25 nt, Artificial Sequence
  Synthetic Polynucleotide
gcaccaguuacaggugcuuuacaag

SEQ ID NO: 17 - RNA, 25 nt, Artificial Sequence
  Synthetic Polynucleotide
caccaguuacaggugcuuuacaagg

SEQ ID NO: 18 - RNA, 25 nt, Artificial Sequence
  Synthetic Polynucleotide
ccaguuacaggugcuuuacaaggcc

SEQ ID NO: 19 - RNA, 25 nt, Artificial Sequence
  Synthetic Polynucleotide
caguuacaggugcuuuacaaggcca

SEQ ID NO: 20 - RNA, 25 nt, Artificial Sequence
  Synthetic Polynucleotide
aguuacaggugcuuuacaaggccaa

SEQ ID NO: 21 - DNA, 25 bp, Artificial Sequence
  Synthetic Polynucleotide
uacaggugcuuuacaaggccaagta

SEQ ID NO: 22 - RNA, 25 nt, Artificial Sequence
  Synthetic Polynucleotide
acaggugcuuuacaaggccaaguac

SEQ ID NO: 23 - RNA, 25 nt, Artificial Sequence
  Synthetic Polynucleotide
caggugcuuuacaaggccaaguacg

SEQ ID NO: 24 - DNA, 25 bp, Artificial Sequence
  Synthetic Polynucleotide
aaguacgguccaauguggaugucct

SEQ ID NO: 25 - RNA, 25 nt, Artificial Sequence
  Synthetic Polynucleotide
guacgguccaauguggauguccuac

SEQ ID NO: 26 - DNA, 25 bp, Artificial Sequence
  Synthetic Polynucleotide
acgguccaauguggauguccuactt

SEQ ID NO: 27 - DNA, 25 bp, Artificial Sequence
  Synthetic Polynucleotide
cgguccaauguggauguccuacuta

SEQ ID NO: 28 - RNA, 25 nt, Artificial Sequence
  Synthetic Polynucleotide
gguccaauguggauguccuacuuag

SEQ ID NO: 29 - RNA, 25 nt, Artificial Sequence
  Synthetic Polynucleotide
ggcaaguacccaguacggaacgaca

SEQ ID NO: 30 - RNA, 25 nt, Artificial Sequence
  Synthetic Polynucleotide
aguacccaguacggaacgacaugga

SEQ ID NO: 31 - RNA, 25 nt, Artificial Sequence
  Synthetic Polynucleotide
guacccaguacggaacgacauggag

SEQ ID NO: 32 - DNA, 25 bp, Artificial Sequence
  Synthetic Polynucleotide
caguacggaacgacauggagcuatg

SEQ ID NO: 33 - RNA, 25 nt, Artificial Sequence
  Synthetic Polynucleotide
guacggaacgacauggagcuaugga

SEQ ID NO: 34 - RNA, 25 nt, Artificial Sequence
  Synthetic Polynucleotide
uacggaacgacauggagcuauggaa

SEQ ID NO: 35 - RNA, 25 nt, Artificial Sequence
  Synthetic Polynucleotide
acggaacgacauggagcuauggaag

SEQ ID NO: 36 - RNA, 25 nt, Artificial Sequence
  Synthetic Polynucleotide
cugaaccagcgguugcugaagccag

SEQ ID NO: 37 - RNA, 25 nt, Artificial Sequence
  Synthetic Polynucleotide
cagcgcucuauacggaugcuuucaa

SEQ ID NO: 38 - DNA, 25 bp, Artificial Sequence
  Synthetic Polynucleotide
agcgcucuauacggaugcuuucaat

SEQ ID NO: 39 - DNA, 25 bp, Artificial Sequence
  Synthetic Polynucleotide
gcgcucuauacggaugcuuucaatg

SEQ ID NO: 40 - RNA, 25 nt, Artificial Sequence
  Synthetic Polynucleotide
cgcucuauacggaugcuuucaauga

SEQ ID NO: 41 - RNA, 25 nt, Artificial Sequence
  Synthetic Polynucleotide
gcucuauacggaugcuuucaaugag

SEQ ID NO: 42 - RNA, 25 nt, Artificial Sequence
  Synthetic Polynucleotide
cucuauacggaugcuuucaaugagg

SEQ ID NO: 43 - DNA, 25 bp, Artificial Sequence
  Synthetic Polynucleotide
ucuauacggaugcuuucaaugaggt

SEQ ID NO: 44 - DNA, 25 bp, Artificial Sequence
  Synthetic Polynucleotide
cuauacggaugcuuucaaugaggtg

SEQ ID NO: 45 - RNA, 25 nt, Artificial Sequence
  Synthetic Polynucleotide
uauacggaugcuuucaaugagguga

SEQ ID NO: 46 - DNA, 25 bp, Artificial Sequence
  Synthetic Polynucleotide
auacggaugcuuucaaugaggugat

SEQ ID NO: 47 - DNA, 25 bp, Artificial Sequence
  Synthetic Polynucleotide
uacggaugcuuucaaugaggugatt

SEQ ID NO: 48 - DNA, 25 bp, Artificial Sequence
  Synthetic Polynucleotide
acggaugcuuucaaugaggugautg

SEQ ID NO: 49 - RNA, 25 nt, Artificial Sequence
  Synthetic Polynucleotide
cggaugcuuucaaugaggugauuga

SEQ ID NO: 50 - DNA, 25 bp, Artificial Sequence
  Synthetic Polynucleotide
ggaugcuuucaaugaggugauugat

SEQ ID NO: 51 - DNA, 25 bp, Artificial Sequence
  Synthetic Polynucleotide
gaugcuuucaaugaggugauugatg

SEQ ID NO: 52 - RNA, 25 nt, Artificial Sequence
  Synthetic Polynucleotide
augcuuucaaugaggugauugauga

SEQ ID NO: 53 - RNA, 25 nt, Artificial Sequence
  Synthetic Polynucleotide
ugcuuucaaugaggugauugaugac

SEQ ID NO: 54 - DNA, 25 bp, Artificial Sequence
  Synthetic Polynucleotide
gcuuucaaugaggugauugaugact

SEQ ID NO: 55 - DNA, 25 bp, Artificial Sequence
  Synthetic Polynucleotide
cuuucaaugaggugauugaugactt

SEQ ID NO: 56 - DNA, 25 bp, Artificial Sequence
  Synthetic Polynucleotide
uuucaaugaggugauugaugacutt

SEQ ID NO: 57 - DNA, 25 bp, Artificial Sequence
  Synthetic Polynucleotide
uucaaugaggugauugaugacuuta

SEQ ID NO: 58 - DNA, 25 bp, Artificial Sequence
  Synthetic Polynucleotide
ucaaugaggugauugaugacuuuat

SEQ ID NO: 59 - DNA, 25 bp, Artificial Sequence
  Synthetic Polynucleotide
caaugaggugauugaugacuuuatg

SEQ ID NO: 60 - RNA, 25 nt, Artificial Sequence
  Synthetic Polynucleotide
aaugaggugauugaugacuuuauga

SEQ ID NO: 61 - RNA, 25 nt, Artificial Sequence
  Synthetic Polynucleotide
augaggugauugaugacuuuaugac

SEQ ID NO: 62 - DNA, 25 bp, Artificial Sequence
  Synthetic Polynucleotide
ugaggugauugaugacuuuaugact

SEQ ID NO: 63 - DNA, 25 bp, Artificial Sequence
  Synthetic Polynucleotide
gaggugauugaugacuuuaugactc

SEQ ID NO: 64 - RNA, 25 nt, Artificial Sequence
  Synthetic Polynucleotide
aggugauugaugacuuuaugacucg

SEQ ID NO: 65 - RNA, 25 nt, Artificial Sequence
  Synthetic Polynucleotide
ggugauugaugacuuuaugacucga

SEQ ID NO: 66 - RNA, 25 nt, Artificial Sequence
  Synthetic Polynucleotide
gugauugaugacuuuaugacucgac

SEQ ID NO: 67 - DNA, 25 bp, Artificial Sequence
  Synthetic Polynucleotide
ugauugaugacuuuaugacucgact

SEQ ID NO: 68 - DNA, 25 bp, Artificial Sequence
  Synthetic Polynucleotide
gauugaugacuuuaugacucgactg

SEQ ID NO: 69 - RNA, 25 nt, Artificial Sequence
  Synthetic Polynucleotide
auugaugacuuuaugacucgacugg

SEQ ID NO: 70 - RNA, 25 nt, Artificial Sequence
  Synthetic Polynucleotide
uugaugacuuuaugacucgacugga

SEQ ID NO: 71 - RNA, 25 nt, Artificial Sequence
  Synthetic Polynucleotide
gaugacuuuaugacucgacuggacc

SEQ ID NO: 72 - RNA, 25 nt, Artificial Sequence
  Synthetic Polynucleotide
augacuuuaugacucgacuggacca

SEQ ID NO: 73 - RNA, 25 nt, Artificial Sequence
  Synthetic Polynucleotide
gacuuuaugacucgacuggaccagc

SEQ ID NO: 74 - DNA, 25 bp, Artificial Sequence
  Synthetic Polynucleotide
acuuuaugacucgacuggaccagct

SEQ ID NO: 75 - DNA, 25 bp, Artificial Sequence
  Synthetic Polynucleotide
ucggacauggcucaacucuucuact

SEQ ID NO: 76 - RNA, 25 nt, Artificial Sequence
  Synthetic Polynucleotide
ggacauggcucaacucuucuacuac

SEQ ID NO: 77 - DNA, 25 bp, Artificial Sequence
  Synthetic Polynucleotide
gacauggcucaacucuucuacuact

SEQ ID NO: 78 - DNA, 25 bp, Artificial Sequence
  Synthetic Polynucleotide
cucaacucuucuacuacuuugcctt

SEQ ID NO: 79 - RNA, 25 nt, Artificial Sequence
  Synthetic Polynucleotide
aacucuucuacuacuuugccuugga

SEQ ID NO: 80 - RNA, 25 nt, Artificial Sequence
  Synthetic Polynucleotide
acucuucuacuacuuugccuuggaa

SEQ ID NO: 81 - RNA, 25 nt, Artificial Sequence
  Synthetic Polynucleotide
cucuucuacuacuuugccuuggaag

SEQ ID NO: 82 - DNA, 25 bp, Artificial Sequence
  Synthetic Polynucleotide
uucuacuacuuugccuuggaagcta

SEQ ID NO: 83 - DNA, 25 bp, Artificial Sequence
  Synthetic Polynucleotide
ucuacuacuuugccuuggaagcuat

SEQ ID NO: 84 - DNA, 25 bp, Artificial Sequence
  Synthetic Polynucleotide
cuacuacuuugccuuggaagcuatt

SEQ ID NO: 85 - DNA, 25 bp, Artificial Sequence
  Synthetic Polynucleotide
uacuacuuugccuuggaagcuautt

SEQ ID NO: 86 - DNA, 25 bp, Artificial Sequence
  Synthetic Polynucleotide
acuacuuugccuuggaagcuauutg

SEQ ID NO: 87 - RNA, 25 nt, Artificial Sequence
  Synthetic Polynucleotide
cuacuuugccuuggaagcuauuugc

SEQ ID NO: 88 - DNA, 25 bp, Artificial Sequence
  Synthetic Polynucleotide
uacuuugccuuggaagcuauuugct

SEQ ID NO: 89 - DNA, 25 bp, Artificial Sequence
  Synthetic Polynucleotide
acuuugccuuggaagcuauuugcta

SEQ ID NO: 90 - RNA, 25 nt, Artificial Sequence
  Synthetic Polynucleotide
cuuugccuuggaagcuauuugcuac

SEQ ID NO: 91 - RNA, 25 nt, Artificial Sequence
  Synthetic Polynucleotide
uuugccuuggaagcuauuugcuaca

SEQ ID NO: 92 - DNA, 25 bp, Artificial Sequence
  Synthetic Polynucleotide
uugccuuggaagcuauuugcuacat

SEQ ID NO: 93 - DNA, 25 bp, Artificial Sequence
  Synthetic Polynucleotide
ugccuuggaagcuauuugcuacatc

SEQ ID NO: 94 - RNA, 25 nt, Artificial Sequence
  Synthetic Polynucleotide
gccuuggaagcuauuugcuacaucc

SEQ ID NO: 95 - DNA, 25 bp, Artificial Sequence
  Synthetic Polynucleotide
ccuuggaagcuauuugcuacaucct

SEQ ID NO: 96 - DNA, 25 bp, Artificial Sequence
  Synthetic Polynucleotide
cuuggaagcuauuugcuacaucctg

SEQ ID NO: 97 - DNA, 25 bp, Artificial Sequence
  Synthetic Polynucleotide
uuggaagcuauuugcuacauccugt

SEQ ID NO: 98 - DNA, 25 bp, Artificial Sequence
  Synthetic Polynucleotide
uggaagcuauuugcuacauccugtt

SEQ ID NO: 99 - DNA, 25 bp, Artificial Sequence
  Synthetic Polynucleotide
ggaagcuauuugcuacauccugutc

SEQ ID NO: 100 - RNA, 25 nt, Artificial Sequence
  Synthetic Polynucleotide
gaagcuauuugcuacauccuguucg

SEQ ID NO: 101 - RNA, 25 nt, Artificial Sequence
  Synthetic Polynucleotide
aagcuauuugcuacauccuguucga

SEQ ID NO: 102 - RNA, 25 nt, Artificial Sequence
  Synthetic Polynucleotide
agcuauuugcuacauccuguucgag

SEQ ID NO: 103 - RNA, 25 nt, Artificial Sequence
  Synthetic Polynucleotide
gcuauuugcuacauccuguucgaga

SEQ ID NO: 104 - RNA, 25 nt, Artificial Sequence
  Synthetic Polynucleotide
cuauuugcuacauccuguucgagaa

SEQ ID NO: 105 - RNA, 25 nt, Artificial Sequence
  Synthetic Polynucleotide
uauuugcuacauccuguucgagaaa

SEQ ID NO: 106 - RNA, 25 nt, Artificial Sequence
  Synthetic Polynucleotide
auuugcuacauccuguucgagaaac

SEQ ID NO: 107 - RNA, 25 nt, Artificial Sequence
  Synthetic Polynucleotide
uuugcuacauccuguucgagaaacg

SEQ ID NO: 108 - RNA, 25 nt, Artificial Sequence
  Synthetic Polynucleotide
uugcuacauccuguucgagaaacgc

SEQ ID NO: 109 - RNA, 25 nt, Artificial Sequence
  Synthetic Polynucleotide
ugcuacauccuguucgagaaacgca

SEQ ID NO: 110 - DNA, 25 bp, Artificial Sequence
  Synthetic Polynucleotide
gcuacauccuguucgagaaacgcat

SEQ ID NO: 111 - DNA, 25 bp, Artificial Sequence
  Synthetic Polynucleotide
cuacauccuguucgagaaacgcatt

SEQ ID NO: 112 - RNA, 25 nt, Artificial Sequence
  Synthetic Polynucleotide
gucagauccaucggguuaauguucc

SEQ ID NO: 113 - RNA, 25 nt, Artificial Sequence
  Synthetic Polynucleotide
ucagauccaucggguuaauguucca

SEQ ID NO: 114 - RNA, 25 nt, Artificial Sequence
  Synthetic Polynucleotide
cagauccaucggguuaauguuccag

SEQ ID NO: 115 - RNA, 25 nt, Artificial Sequence
  Synthetic Polynucleotide
agauccaucggguuaauguuccaga

SEQ ID NO: 116 - RNA, 25 nt, Artificial Sequence
  Synthetic Polynucleotide
gauccaucggguuaauguuccagaa

SEQ ID NO: 117 - RNA, 25 nt, Artificial Sequence
  Synthetic Polynucleotide
auccaucggguuaauguuccagaac

SEQ ID NO: 118 - DNA, 25 bp, Artificial Sequence
  Synthetic Polynucleotide
uccaucggguuaauguuccagaact

SEQ ID NO: 119 - DNA, 25 bp, Artificial Sequence
  Synthetic Polynucleotide
ccaucggguuaauguuccagaactc

SEQ ID NO: 120 - RNA, 25 nt, Artificial Sequence
  Synthetic Polynucleotide
caucggguuaauguuccagaacuca

SEQ ID NO: 121 - RNA, 25 nt, Artificial Sequence
  Synthetic Polynucleotide
aucggguuaauguuccagaacucac

SEQ ID NO: 122 - DNA, 25 bp, Artificial Sequence
  Synthetic Polynucleotide
ucggguuaauguuccagaacucact

SEQ ID NO: 123 - DNA, 25 bp, Artificial Sequence
  Synthetic Polynucleotide
cggguuaauguuccagaacucactc

SEQ ID NO: 124 - DNA, 25 bp, Artificial Sequence
  Synthetic Polynucleotide
ggguuaauguuccagaacucacuct

SEQ ID NO: 125 - DNA, 25 bp, Artificial Sequence
  Synthetic Polynucleotide
gguuaauguuccagaacucacucta

SEQ ID NO: 126 - DNA, 25 bp, Artificial Sequence
  Synthetic Polynucleotide
guuaauguuccagaacucacucuat

SEQ ID NO: 127 - DNA, 25 bp, Artificial Sequence
  Synthetic Polynucleotide
uuaauguuccagaacucacucuatg

SEQ ID NO: 128 - RNA, 25 nt, Artificial Sequence
  Synthetic Polynucleotide
uaauguuccagaacucacucuaugc

SEQ ID NO: 129 - RNA, 25 nt, Artificial Sequence
  Synthetic Polynucleotide
aauguuccagaacucacucuaugcc

SEQ ID NO: 130 - RNA, 25 nt, Artificial Sequence
  Synthetic Polynucleotide
auguuccagaacucacucuaugcca

SEQ ID NO: 131 - RNA, 25 nt, Artificial Sequence
  Synthetic Polynucleotide
uguuccagaacucacucuaugccac

SEQ ID NO: 132 - RNA, 25 nt, Artificial Sequence
  Synthetic Polynucleotide
guuccagaacucacucuaugccacc

SEQ ID NO: 133 - DNA, 25 bp, Artificial Sequence
  Synthetic Polynucleotide
ccagaacucacucuaugccaccutc

SEQ ID NO: 134 - RNA, 25 nt, Artificial Sequence
  Synthetic Polynucleotide
cagaacucacucuaugccaccuucc

SEQ ID NO: 135 - RNA, 25 nt, Artificial Sequence
  Synthetic Polynucleotide
ggaagcgauaccuggaugguuggaa

SEQ ID NO: 136 - DNA, 25 bp, Artificial Sequence
  Synthetic Polynucleotide
gaagcgauaccuggaugguuggaat

SEQ ID NO: 137 - DNA, 25 bp, Artificial Sequence
  Synthetic Polynucleotide
aagcgauaccuggaugguuggaatg

SEQ ID NO: 138 - RNA, 25 nt, Artificial Sequence
  Synthetic Polynucleotide
agcgauaccuggaugguuggaaugc

SEQ ID NO: 139 - RNA, 25 nt, Artificial Sequence
  Synthetic Polynucleotide
gcgauaccuggaugguuggaaugcc

SEQ ID NO: 140 - RNA, 25 nt, Artificial Sequence
  Synthetic Polynucleotide
cgauaccuggaugguuggaaugcca

SEQ ID NO: 141 - DNA, 25 bp, Artificial Sequence
  Synthetic Polynucleotide
gauaccuggaugguuggaaugccat

SEQ ID NO: 142 - DNA, 25 bp, Artificial Sequence
  Synthetic Polynucleotide
auaccuggaugguuggaaugccatc

SEQ ID NO: 143 - DNA, 25 bp, Artificial Sequence
  Synthetic Polynucleotide
accuggaugguuggaaugccauctt

SEQ ID NO: 144 - DNA, 25 bp, Artificial Sequence
  Synthetic Polynucleotide
ccuggaugguuggaaugccaucutt

SEQ ID NO: 145 - DNA, 25 bp, Artificial Sequence
  Synthetic Polynucleotide
cuggaugguuggaaugccaucuutt

SEQ ID NO: 146 - DNA, 25 bp, Artificial Sequence
  Synthetic Polynucleotide
uggaugguuggaaugccaucuuutc

SEQ ID NO: 147 - RNA, 25 nt, Artificial Sequence
  Synthetic Polynucleotide
ggaugguuggaaugccaucuuuucc

SEQ ID NO: 148 - DNA, 25 bp, Artificial Sequence
  Synthetic Polynucleotide
gaugguuggaaugccaucuuuucct

SEQ ID NO: 149 - DNA, 25 bp, Artificial Sequence
  Synthetic Polynucleotide
augguuggaaugccaucuuuucctt

SEQ ID NO: 150 - DNA, 25 bp, Artificial Sequence
  Synthetic Polynucleotide
ugguuggaaugccaucuuuuccutt

SEQ ID NO: 151 - DNA, 25 bp, Artificial Sequence
  Synthetic Polynucleotide
gguuggaaugccaucuuuuccuutg

SEQ ID NO: 152 - RNA, 25 nt, Artificial Sequence
  Synthetic Polynucleotide
guuggaaugccaucuuuuccuuugg

SEQ ID NO: 153 - RNA, 25 nt, Artificial Sequence
  Synthetic Polynucleotide
ggaaugccaucuuuuccuuugggaa

SEQ ID NO: 154 - RNA, 25 nt, Artificial Sequence
  Synthetic Polynucleotide
ccuuugggaagaagcugauugauga

SEQ ID NO: 155 - RNA, 25 nt, Artificial Sequence
  Synthetic Polynucleotide
gggaagaagcugauugaugagaagc

SEQ ID NO: 156 - DNA, 25 bp, Artificial Sequence
  Synthetic Polynucleotide
ggaagaagcugauugaugagaagct

SEQ ID NO: 157 - DNA, 25 bp, Artificial Sequence
  Synthetic Polynucleotide
gaagaagcugauugaugagaagctc

SEQ ID NO: 158 - RNA, 25 nt, Artificial Sequence
  Synthetic Polynucleotide
aagaagcugauugaugagaagcucg

SEQ ID NO: 159 - RNA, 25 nt, Artificial Sequence
  Synthetic Polynucleotide
agaagcugauugaugagaagcucga

SEQ ID NO: 160 - RNA, 25 nt, Artificial Sequence
  Synthetic Polynucleotide
gaagcugauugaugagaagcucgaa

SEQ ID NO: 161 - RNA, 25 nt, Artificial Sequence
  Synthetic Polynucleotide
aagcugauugaugagaagcucgaag

SEQ ID NO: 162 - RNA, 25 nt, Artificial Sequence
  Synthetic Polynucleotide
agcugauugaugagaagcucgaaga

SEQ ID NO: 163 - DNA, 25 bp, Artificial Sequence
  Synthetic Polynucleotide
gcugauugaugagaagcucgaagat

SEQ ID NO: 164 - DNA, 25 bp, Artificial Sequence
  Synthetic Polynucleotide
cugauugaugagaagcucgaagata

SEQ ID NO: 165 - DNA, 25 bp, Artificial Sequence
  Synthetic Polynucleotide
ugauugaugagaagcucgaagauat

SEQ ID NO: 166 - DNA, 25 bp, Artificial Sequence
  Synthetic Polynucleotide
gauugaugagaagcucgaagauatg

SEQ ID NO: 167 - RNA, 25 nt, Artificial Sequence
  Synthetic Polynucleotide
auugaugagaagcucgaagauaugg

SEQ ID NO: 168 - RNA, 25 nt, Artificial Sequence
  Synthetic Polynucleotide
uugaugagaagcucgaagauaugga

SEQ ID NO: 169 - RNA, 25 nt, Artificial Sequence
  Synthetic Polynucleotide
ugaugagaagcucgaagauauggag

SEQ ID NO: 170 - RNA, 25 nt, Artificial Sequence
  Synthetic Polynucleotide
gaugagaagcucgaagauauggagg

SEQ ID NO: 171 - DNA, 25 bp, Artificial Sequence
  Synthetic Polynucleotide
cugacaugggcccuguaccaccuct

SEQ ID NO: 172 - DNA, 25 bp, Artificial Sequence
  Synthetic Polynucleotide
ugacaugggcccuguaccaccuctc

SEQ ID NO: 173 - RNA, 25 nt, Artificial Sequence
  Synthetic Polynucleotide
gacaugggcccuguaccaccucuca

SEQ ID NO: 174 - RNA, 25 nt, Artificial Sequence
  Synthetic Polynucleotide
acaugggcccuguaccaccucucaa

SEQ ID NO: 175 - RNA, 25 nt, Artificial Sequence
  Synthetic Polynucleotide
caugggcccuguaccaccucucaaa

SEQ ID NO: 176 - RNA, 25 nt, Artificial Sequence
  Synthetic Polynucleotide
gagauccaggaggccuugcacgagg

SEQ ID NO: 177 - RNA, 25 nt, Artificial Sequence
  Synthetic Polynucleotide
agauccaggaggccuugcacgagga

SEQ ID NO: 178 - RNA, 25 nt, Artificial Sequence
  Synthetic Polynucleotide
gauccaggaggccuugcacgaggaa

SEQ ID NO: 179 - RNA, 25 nt, Artificial Sequence
  Synthetic Polynucleotide
gugccccagcacaaggacuuugccc

SEQ ID NO: 180 - RNA, 25 nt, Artificial Sequence
  Synthetic Polynucleotide
ugccccagcacaaggacuuugccca

SEQ ID NO: 181 - RNA, 25 nt, Artificial Sequence
  Synthetic Polynucleotide
gccccagcacaaggacuuugcccac

SEQ ID NO: 182 - RNA, 25 nt, Artificial Sequence
  Synthetic Polynucleotide
ccccagcacaaggacuuugcccaca

SEQ ID NO: 183 - DNA, 25 bp, Artificial Sequence
  Synthetic Polynucleotide
cccagcacaaggacuuugcccacat

SEQ ID NO: 184 - DNA, 25 bp, Artificial Sequence
  Synthetic Polynucleotide
ccagcacaaggacuuugcccacatg

SEQ ID NO: 185 - RNA, 25 nt, Artificial Sequence
  Synthetic Polynucleotide
cagcacaaggacuuugcccacaugc

SEQ ID NO: 186 - RNA, 25 nt, Artificial Sequence
  Synthetic Polynucleotide
agcacaaggacuuugcccacaugcc

SEQ ID NO: 187 - RNA, 25 nt, Artificial Sequence
  Synthetic Polynucleotide
gcacaaggacuuugcccacaugccg

SEQ ID NO: 188 - DNA, 25 bp, Artificial Sequence
  Synthetic Polynucleotide
cacaaggacuuugcccacaugccgt

SEQ ID NO: 189 - DNA, 25 bp, Artificial Sequence
  Synthetic Polynucleotide
acaaggacuuugcccacaugccgtt

SEQ ID NO: 190 - DNA, 25 bp, Artificial Sequence
  Synthetic Polynucleotide
caaggacuuugcccacaugccgutg

SEQ ID NO: 191 - RNA, 25 nt, Artificial Sequence
  Synthetic Polynucleotide
aaggacuuugcccacaugccguugc

SEQ ID NO: 192 - DNA, 25 bp, Artificial Sequence
  Synthetic Polynucleotide
aggacuuugcccacaugccguugct

SEQ ID NO: 193 - DNA, 25 bp, Artificial Sequence
  Synthetic Polynucleotide
cucaaagcugugcuuaaggagactc

SEQ ID NO: 194 - DNA, 25 bp, Artificial Sequence
  Synthetic Polynucleotide
caaagcugugcuuaaggagacuctg

SEQ ID NO: 195 - RNA, 25 nt, Artificial Sequence
  Synthetic Polynucleotide
cccacaaacucccggaucauagaaa

SEQ ID NO: 196 - RNA, 25 nt, Artificial Sequence
  Synthetic Polynucleotide
ccacaaacucccggaucauagaaaa

SEQ ID NO: 197 - RNA, 25 nt, Artificial Sequence
  Synthetic Polynucleotide
acaaacucccggaucauagaaaagg

SEQ ID NO: 198 - RNA, 25 nt, Artificial Sequence
  Synthetic Polynucleotide
caaacucccggaucauagaaaagga

SEQ ID NO: 199 - DNA, 25 bp, Artificial Sequence
  Synthetic Polynucleotide
ucccggaucauagaaaaggaaautg

SEQ ID NO: 200 - RNA, 25 nt, Artificial Sequence
  Synthetic Polynucleotide
cccggaucauagaaaaggaaauuga

SEQ ID NO: 201 - RNA, 25 nt, Artificial Sequence
  Synthetic Polynucleotide
ccggaucauagaaaaggaaauugaa

SEQ ID NO: 202 - RNA, 25 nt, Artificial Sequence
  Synthetic Polynucleotide
cggaucauagaaaaggaaauugaag

SEQ ID NO: 203 - DNA, 25 bp, Artificial Sequence
  Synthetic Polynucleotide
ggaucauagaaaaggaaauugaagt

SEQ ID NO: 204 - DNA, 25 bp, Artificial Sequence
  Synthetic Polynucleotide
gaucauagaaaaggaaauugaagtt

SEQ ID NO: 205 - DNA, 25 bp, Artificial Sequence
  Synthetic Polynucleotide
aucauagaaaaggaaauugaagutg

SEQ ID NO: 206 - RNA, 25 nt, Artificial Sequence
  Synthetic Polynucleotide
ucauagaaaaggaaauugaaguuga

SEQ ID NO: 207 - DNA, 25 bp, Artificial Sequence
  Synthetic Polynucleotide
cauagaaaaggaaauugaaguugat

SEQ ID NO: 208 - DNA, 25 bp, Artificial Sequence
  Synthetic Polynucleotide
auagaaaaggaaauugaaguugatg

SEQ ID NO: 209 - RNA, 25 nt, Artificial Sequence
  Synthetic Polynucleotide
uagaaaaggaaauugaaguugaugg

SEQ ID NO: 210 - RNA, 25 nt, Artificial Sequence
  Synthetic Polynucleotide
agaaaaggaaauugaaguugauggc

SEQ ID NO: 211 - DNA, 25 bp, Artificial Sequence
  Synthetic Polynucleotide
gaaaaggaaauugaaguugauggct

SEQ ID NO: 212 - DNA, 25 bp, Artificial Sequence
  Synthetic Polynucleotide
aaaaggaaauugaaguugauggctt

SEQ ID NO: 213 - DNA, 25 bp, Artificial Sequence
  Synthetic Polynucleotide
aaaggaaauugaaguugauggcutc

SEQ ID NO: 214 - DNA, 25 bp, Artificial Sequence
  Synthetic Polynucleotide
ggaaauugaaguugauggcuucctc

SEQ ID NO: 215 - DNA, 25 bp, Artificial Sequence
  Synthetic Polynucleotide
gaaauugaaguugauggcuuccuct

SEQ ID NO: 216 - DNA, 25 bp, Artificial Sequence
  Synthetic Polynucleotide
aaauugaaguugauggcuuccuctt

SEQ ID NO: 217 - RNA, 25 nt, Artificial Sequence
  Synthetic Polynucleotide
gcaaggcugauccagaaguacaagg

SEQ ID NO: 218 - DNA, 25 bp, Artificial Sequence
  Synthetic Polynucleotide
caaggcugauccagaaguacaaggt

SEQ ID NO: 219 - DNA, 25 bp, Artificial Sequence
  Synthetic Polynucleotide
aaggcugauccagaaguacaaggtg

SEQ ID NO: 220 - RNA, 25 nt, Artificial Sequence
  Synthetic Polynucleotide
aggcugauccagaaguacaaggugg

SEQ ID NO: 221 - RNA, 25 nt, Artificial Sequence
  Synthetic Polynucleotide
cgcauuguccugguucccaauaaga

SEQ ID NO: 222 - RNA, 25 nt, Artificial Sequence
  Synthetic Polynucleotide
gcauuguccugguucccaauaagaa

SEQ ID NO: 223 - RNA, 25 nt, Artificial Sequence
  Synthetic Polynucleotide
cauuguccugguucccaauaagaaa

SEQ ID NO: 224 - RNA, 25 nt, Artificial Sequence
  Synthetic Polynucleotide
auuguccugguucccaauaagaaag

SEQ ID NO: 225 - DNA, 25 bp, Artificial Sequence
  Synthetic Polynucleotide
uuguccugguucccaauaagaaagt

SEQ ID NO: 226 - DNA, 25 bp, Artificial Sequence
  Synthetic Polynucleotide
uguccugguucccaauaagaaagtg

SEQ ID NO: 227 - RNA, 25 nt, Artificial Sequence
  Synthetic Polynucleotide
guccugguucccaauaagaaagugg

SEQ ID NO: 228 - DNA, 25 bp, Artificial Sequence
  Synthetic Polynucleotide
acccugagcuuuugccacuucuatc

SEQ ID NO: 229 - RNA, 25 nt, Artificial Sequence
  Synthetic Polynucleotide
cccugagcuuuugccacuucuauca

SEQ ID NO: 230 - DNA, 25 bp, Artificial Sequence
  Synthetic Polynucleotide
ccugagcuuuugccacuucuaucat

SEQ ID NO: 231 - DNA, 25 bp, Artificial Sequence
  Synthetic Polynucleotide
cugagcuuuugccacuucuaucatt

SEQ ID NO: 232 - DNA, 25 bp, Artificial Sequence
  Synthetic Polynucleotide
ugagcuuuugccacuucuaucautt

SEQ ID NO: 233 - DNA, 25 bp, Artificial Sequence
  Synthetic Polynucleotide
gagcuuuugccacuucuaucauutt

SEQ ID NO: 234 - DNA, 25 bp, Artificial Sequence
  Synthetic Polynucleotide
agcuuuugccacuucuaucauuutt

SEQ ID NO: 235 - DNA, 25 bp, Artificial Sequence
  Synthetic Polynucleotide
gcuuuugccacuucuaucauuuutg

SEQ ID NO: 236 - RNA, 25 nt, Artificial Sequence
  Synthetic Polynucleotide
cuuuugccacuucuaucauuuuuga

SEQ ID NO: 237 - RNA, 25 nt, Artificial Sequence
  Synthetic Polynucleotide
uuuugccacuucuaucauuuuugag

SEQ ID NO: 238 - RNA, 25 nt, Artificial Sequence
  Synthetic Polynucleotide
uuugccacuucuaucauuuuugagc

SEQ ID NO: 239 - RNA, 25 nt, Artificial Sequence
  Synthetic Polynucleotide
uugccacuucuaucauuuuugagca

SEQ ID NO: 240 - RNA, 25 nt, Artificial Sequence
  Synthetic Polynucleotide
ugccacuucuaucauuuuugagcaa

SEQ ID NO: 241 - RNA, 25 nt, Artificial Sequence
  Synthetic Polynucleotide
gccacuucuaucauuuuugagcaac

SEQ ID NO: 242 - DNA, 25 bp, Artificial Sequence
  Synthetic Polynucleotide
ccacuucuaucauuuuugagcaact

SEQ ID NO: 243 - DNA, 25 bp, Artificial Sequence
  Synthetic Polynucleotide
cacuucuaucauuuuugagcaactc

SEQ ID NO: 244 - RNA, 25 nt, Artificial Sequence
  Synthetic Polynucleotide
acuucuaucauuuuugagcaacucc

SEQ ID NO: 245 - RNA, 25 nt, Artificial Sequence
  Synthetic Polynucleotide
cuucuaucauuuuugagcaacuccc

SEQ ID NO: 246 - DNA, 25 bp, Artificial Sequence
  Synthetic Polynucleotide
uucuaucauuuuugagcaacuccct

SEQ ID NO: 247 - DNA, 25 bp, Artificial Sequence
  Synthetic Polynucleotide
ucuaucauuuuugagcaacuccctc

SEQ ID NO: 248 - DNA, 25 bp, Artificial Sequence
  Synthetic Polynucleotide
cuaucauuuuugagcaacucccuct

SEQ ID NO: 249 - RNA, 25 nt, Artificial Sequence
  Synthetic Polynucleotide
aucauuuuugagcaacucccucuca

SEQ ID NO: 250 - RNA, 25 nt, Artificial Sequence
  Synthetic Polynucleotide
ucauuuuugagcaacucccucucag

SEQ ID NO: 251 - RNA, 25 nt, Artificial Sequence
  Synthetic Polynucleotide
gagcaacucccucucagcuaaaagg

SEQ ID NO: 252 - DNA, 25 bp, Artificial Sequence
  Synthetic Polynucleotide
cgcauugcuguccuuggguagaata

SEQ ID NO: 253 - DNA, 25 bp, Artificial Sequence
  Synthetic Polynucleotide
gcauugcuguccuuggguagaauat

SEQ ID NO: 254 - DNA, 25 bp, Artificial Sequence
  Synthetic Polynucleotide
cauugcuguccuuggguagaauata

SEQ ID NO: 255 - RNA, 25 nt, Artificial Sequence
  Synthetic Polynucleotide
auugcuguccuuggguagaauauaa

SEQ ID NO: 256 - RNA, 25 nt, Artificial Sequence
  Synthetic Polynucleotide
uugcuguccuuggguagaauauaaa

SEQ ID NO: 257 - RNA, 25 nt, Artificial Sequence
  Synthetic Polynucleotide
ugcuguccuuggguagaauauaaaa

SEQ ID NO: 258 - DNA, 25 bp, Artificial Sequence
  Synthetic Polynucleotide
gcuguccuuggguagaauauaaaat

SEQ ID NO: 259 - DNA, 25 bp, Artificial Sequence
  Synthetic Polynucleotide
cuguccuuggguagaauauaaaata

SEQ ID NO: 260 - RNA, 25 nt, Artificial Sequence
  Synthetic Polynucleotide
uguccuuggguagaauauaaaauaa

SEQ ID NO: 261 - RNA, 25 nt, Artificial Sequence
  Synthetic Polynucleotide
guccuuggguagaauauaaaauaaa

SEQ ID NO: 262 - RNA, 25 nt, Artificial Sequence
  Synthetic Polynucleotide
uccuuggguagaauauaaaauaaag

SEQ ID NO: 263 - RNA, 25 nt, Artificial Sequence
  Synthetic Polynucleotide
ccuuggguagaauauaaaauaaagg

SEQ ID NO: 264 - RNA, 25 nt, Artificial Sequence
  Synthetic Polynucleotide
cuuggguagaauauaaaauaaaggg

SEQ ID NO: 265 - RNA, 25 nt, Artificial Sequence
  Synthetic Polynucleotide
uuggguagaauauaaaauaaaggga

SEQ ID NO: 266 - RNA, 25 nt, Artificial Sequence
  Synthetic Polynucleotide
uggguagaauauaaaauaaagggac

SEQ ID NO: 267 - DNA, 25 bp, Artificial Sequence
  Synthetic Polynucleotide
ggguagaauauaaaauaaagggact

SEQ ID NO: 268 - DNA, 25 bp, Artificial Sequence
  Synthetic Polynucleotide
guagaauauaaaauaaagggacutt

SEQ ID NO: 269 - DNA, 25 bp, Artificial Sequence
  Synthetic Polynucleotide
uagaauauaaaauaaagggacuutt

SEQ ID NO: 270 - DNA, 25 bp, Artificial Sequence
  Synthetic Polynucleotide
agaauauaaaauaaagggacuuuta

SEQ ID NO: 271 - DNA, 25 bp, Artificial Sequence
  Synthetic Polynucleotide
gaauauaaaauaaagggacuuuuat

SEQ ID NO: 272 - DNA, 25 bp, Artificial Sequence
  Synthetic Polynucleotide
aauauaaaauaaagggacuuuuatt

SEQ ID NO: 273 - DNA, 25 bp, Artificial Sequence
  Synthetic Polynucleotide
auauaaaauaaagggacuuuuautt

SEQ ID NO: 274 - DNA, 25 bp, Artificial Sequence
  Synthetic Polynucleotide
uauaaaauaaagggacuuuuauutc

SEQ ID NO: 275 - DNA, 25 bp, Artificial Sequence
  Synthetic Polynucleotide
auaaaauaaagggacuuuuauuuct

SEQ ID NO: 276 - DNA, 25 bp, Artificial Sequence
  Synthetic Polynucleotide
uaaaauaaagggacuuuuauuuctt

SEQ ID NO: 277 - DNA, 25 bp, Artificial Sequence
  Synthetic Polynucleotide
aaaauaaagggacuuuuauuucuta

SEQ ID NO: 278 - DNA, 25 bp, Artificial Sequence
  Synthetic Polynucleotide
aaauaaagggacuuuuauuucuuat

SEQ ID NO: 279 - DNA, 25 bp, Artificial Sequence
  Synthetic Polynucleotide
aauaaagggacuuuuauuucuuatt

SEQ ID NO: 280 - DNA, 25 bp, Artificial Sequence
  Synthetic Polynucleotide
auaaagggacuuuuauuucuuautg

SEQ ID NO: 281 - RNA, 25 nt, Artificial Sequence
  Synthetic Polynucleotide
uaaagggacuuuuauuucuuauugg

SEQ ID NO: 282 - RNA, 25 nt, Artificial Sequence
  Synthetic Polynucleotide
aaagggacuuuuauuucuuauugga

SEQ ID NO: 283 - RNA, 25 nt, Artificial Sequence
  Synthetic Polynucleotide
aagggacuuuuauuucuuauuggaa

SEQ ID NO: 284 - RNA, 25 nt, Artificial Sequence
  Synthetic Polynucleotide
agggacuuuuauuucuuauuggaaa

SEQ ID NO: 285 - RNA, 25 nt, Artificial Sequence
  Synthetic Polynucleotide
gggacuuuuauuucuuauuggaaaa

SEQ ID NO: 286 - RNA, 25 nt, Artificial Sequence
  Synthetic Polynucleotide
ggacuuuuauuucuuauuggaaaaa

SEQ ID NO: 287 - RNA, 25 nt, Artificial Sequence
  Synthetic Polynucleotide
gacuuuuauuucuuauuggaaaaaa

SEQ ID NO: 288 - RNA, 25 nt, Artificial Sequence
  Synthetic Polynucleotide
acuuuuauuucuuauuggaaaaaaa

SEQ ID NO: 289 - RNA, 27 nt, Artificial Sequence
  Synthetic Polynucleotide
acuuuucgcuuggucugaacucugggc

SEQ ID NO: 290 - RNA, 27 nt, Artificial Sequence
  Synthetic Polynucleotide
aacuuuucgcuuggucugaacucuggg

SEQ ID NO: 291 - RNA, 27 nt, Artificial Sequence
  Synthetic Polynucleotide
uaacuuuucgcuuggucugaacucugg

SEQ ID NO: 292 - RNA, 27 nt, Artificial Sequence
  Synthetic Polynucleotide
auaacuuuucgcuuggucugaacucug

SEQ ID NO: 293 - RNA, 27 nt, Artificial Sequence
  Synthetic Polynucleotide
aauaacuuuucgcuuggucugaacucu

SEQ ID NO: 294 - RNA, 27 nt, Artificial Sequence
  Synthetic Polynucleotide
aaauaacuuuucgcuuggucugaacuc

SEQ ID NO: 295 - RNA, 27 nt, Artificial Sequence
  Synthetic Polynucleotide
caaauaacuuuucgcuuggucugaacu

SEQ ID NO: 296 - RNA, 27 nt, Artificial Sequence
  Synthetic Polynucleotide
ucaaauaacuuuucgcuuggucugaac

SEQ ID NO: 297 - RNA, 27 nt, Artificial Sequence
  Synthetic Polynucleotide
cucaaauaacuuuucgcuuggucugaa

SEQ ID NO: 298 - RNA, 27 nt, Artificial Sequence
  Synthetic Polynucleotide
ucucaaauaacuuuucgcuuggucuga

SEQ ID NO: 299 - RNA, 27 nt, Artificial Sequence
  Synthetic Polynucleotide
cucucaaauaacuuuucgcuuggucug

SEQ ID NO: 300 - RNA, 27 nt, Artificial Sequence
  Synthetic Polynucleotide
ccucucaaauaacuuuucgcuuggucu

SEQ ID NO: 301 - RNA, 27 nt, Artificial Sequence
  Synthetic Polynucleotide
gccucucaaauaacuuuucgcuugguc

SEQ ID NO: 302 - RNA, 27 nt, Artificial Sequence
  Synthetic Polynucleotide
uguaaagcaccuguaacuggugcaguu

SEQ ID NO: 303 - RNA, 27 nt, Artificial Sequence
  Synthetic Polynucleotide
uuguaaagcaccuguaacuggugcagu

SEQ ID NO: 304 - RNA, 27 nt, Artificial Sequence
  Synthetic Polynucleotide
cuuguaaagcaccuguaacuggugcag

SEQ ID NO: 305 - RNA, 27 nt, Artificial Sequence
  Synthetic Polynucleotide
ccuuguaaagcaccuguaacuggugca

SEQ ID NO: 306 - RNA, 27 nt, Artificial Sequence
  Synthetic Polynucleotide
ggccuuguaaagcaccuguaacuggug

SEQ ID NO: 307 - RNA, 27 nt, Artificial Sequence
  Synthetic Polynucleotide
uggccuuguaaagcaccuguaacuggu

SEQ ID NO: 308 - RNA, 27 nt, Artificial Sequence
  Synthetic Polynucleotide
uuggccuuguaaagcaccuguaacugg

SEQ ID NO: 309 - RNA, 27 nt, Artificial Sequence
  Synthetic Polynucleotide
uacuuggccuuguaaagcaccuguaac

SEQ ID NO: 310 - RNA, 27 nt, Artificial Sequence
  Synthetic Polynucleotide
guacuuggccuuguaaagcaccuguaa

SEQ ID NO: 311 - RNA, 27 nt, Artificial Sequence
  Synthetic Polynucleotide
cguacuuggccuuguaaagcaccugua

SEQ ID NO: 312 - RNA, 27 nt, Artificial Sequence
  Synthetic Polynucleotide
aggacauccacauuggaccguacuugg

SEQ ID NO: 313 - RNA, 27 nt, Artificial Sequence
  Synthetic Polynucleotide
guaggacauccacauuggaccguacuu

SEQ ID NO: 314 - RNA, 27 nt, Artificial Sequence
  Synthetic Polynucleotide
aaguaggacauccacauuggaccguac

SEQ ID NO: 315 - RNA, 27 nt, Artificial Sequence
  Synthetic Polynucleotide
uaaguaggacauccacauuggaccgua

SEQ ID NO: 316 - RNA, 27 nt, Artificial Sequence
  Synthetic Polynucleotide
cuaaguaggacauccacauuggaccgu

SEQ ID NO: 317 - RNA, 27 nt, Artificial Sequence
  Synthetic Polynucleotide
ugucguuccguacuggguacuugcccu

SEQ ID NO: 318 - RNA, 27 nt, Artificial Sequence
  Synthetic Polynucleotide
uccaugucguuccguacuggguacuug

SEQ ID NO: 319 - RNA, 27 nt, Artificial Sequence
  Synthetic Polynucleotide
cuccaugucguuccguacuggguacuu

SEQ ID NO: 320 - RNA, 27 nt, Artificial Sequence
  Synthetic Polynucleotide
cauagcuccaugucguuccguacuggg

SEQ ID NO: 321 - RNA, 27 nt, Artificial Sequence
  Synthetic Polynucleotide
uccauagcuccaugucguuccguacug

SEQ ID NO: 322 - RNA, 27 nt, Artificial Sequence
  Synthetic Polynucleotide
uuccauagcuccaugucguuccguacu

SEQ ID NO: 323 - RNA, 27 nt, Artificial Sequence
  Synthetic Polynucleotide
cuuccauagcuccaugucguuccguac

SEQ ID NO: 324 - RNA, 27 nt, Artificial Sequence
  Synthetic Polynucleotide
cuggcuucagcaaccgcugguucagag

SEQ ID NO: 325 - RNA, 27 nt, Artificial Sequence
  Synthetic Polynucleotide
uugaaagcauccguauagagcgcugcu

SEQ ID NO: 326 - RNA, 27 nt, Artificial Sequence
  Synthetic Polynucleotide
auugaaagcauccguauagagcgcugc

SEQ ID NO: 327 - RNA, 27 nt, Artificial Sequence
  Synthetic Polynucleotide
cauugaaagcauccguauagagcgcug

SEQ ID NO: 328 - RNA, 27 nt, Artificial Sequence
  Synthetic Polynucleotide
ucauugaaagcauccguauagagcgcu

SEQ ID NO: 329 - RNA, 27 nt, Artificial Sequence
  Synthetic Polynucleotide
cucauugaaagcauccguauagagcgc

SEQ ID NO: 330 - RNA, 27 nt, Artificial Sequence
  Synthetic Polynucleotide
ccucauugaaagcauccguauagagcg

SEQ ID NO: 331 - RNA, 27 nt, Artificial Sequence
  Synthetic Polynucleotide
accucauugaaagcauccguauagagc

SEQ ID NO: 332 - RNA, 27 nt, Artificial Sequence
  Synthetic Polynucleotide
caccucauugaaagcauccguauagag

SEQ ID NO: 333 - RNA, 27 nt, Artificial Sequence
  Synthetic Polynucleotide
ucaccucauugaaagcauccguauaga

SEQ ID NO: 334 - RNA, 27 nt, Artificial Sequence
  Synthetic Polynucleotide
aucaccucauugaaagcauccguauag

SEQ ID NO: 335 - RNA, 27 nt, Artificial Sequence
  Synthetic Polynucleotide
aaucaccucauugaaagcauccguaua

SEQ ID NO: 336 - RNA, 27 nt, Artificial Sequence
  Synthetic Polynucleotide
caaucaccucauugaaagcauccguau

SEQ ID NO: 337 - RNA, 27 nt, Artificial Sequence
  Synthetic Polynucleotide
ucaaucaccucauugaaagcauccgua

SEQ ID NO: 338 - RNA, 27 nt, Artificial Sequence
  Synthetic Polynucleotide
aucaaucaccucauugaaagcauccgu

SEQ ID NO: 339 - RNA, 27 nt, Artificial Sequence
  Synthetic Polynucleotide
caucaaucaccucauugaaagcauccg

SEQ ID NO: 340 - RNA, 27 nt, Artificial Sequence
  Synthetic Polynucleotide
ucaucaaucaccucauugaaagcaucc

SEQ ID NO: 341 - RNA, 27 nt, Artificial Sequence
  Synthetic Polynucleotide
gucaucaaucaccucauugaaagcauc

SEQ ID NO: 342 - RNA, 27 nt, Artificial Sequence
  Synthetic Polynucleotide
agucaucaaucaccucauugaaagcau

SEQ ID NO: 343 - RNA, 27 nt, Artificial Sequence
  Synthetic Polynucleotide
aagucaucaaucaccucauugaaagca

SEQ ID NO: 344 - RNA, 27 nt, Artificial Sequence
  Synthetic Polynucleotide
aaagucaucaaucaccucauugaaagc

SEQ ID NO: 345 - RNA, 27 nt, Artificial Sequence
  Synthetic Polynucleotide
uaaagucaucaaucaccucauugaaag

SEQ ID NO: 346 - RNA, 27 nt, Artificial Sequence
  Synthetic Polynucleotide
auaaagucaucaaucaccucauugaaa

SEQ ID NO: 347 - RNA, 27 nt, Artificial Sequence
  Synthetic Polynucleotide
cauaaagucaucaaucaccucauugaa

SEQ ID NO: 348 - RNA, 27 nt, Artificial Sequence
  Synthetic Polynucleotide
ucauaaagucaucaaucaccucauuga

SEQ ID NO: 349 - RNA, 27 nt, Artificial Sequence
  Synthetic Polynucleotide
gucauaaagucaucaaucaccucauug

SEQ ID NO: 350 - RNA, 27 nt, Artificial Sequence
  Synthetic Polynucleotide
agucauaaagucaucaaucaccucauu

SEQ ID NO: 351 - RNA, 27 nt, Artificial Sequence
  Synthetic Polynucleotide
gagucauaaagucaucaaucaccucau

SEQ ID NO: 352 - RNA, 27 nt, Artificial Sequence
  Synthetic Polynucleotide
cgagucauaaagucaucaaucaccuca

SEQ ID NO: 353 - RNA, 27 nt, Artificial Sequence
  Synthetic Polynucleotide
ucgagucauaaagucaucaaucaccuc

SEQ ID NO: 354 - RNA, 27 nt, Artificial Sequence
  Synthetic Polynucleotide
gucgagucauaaagucaucaaucaccu

SEQ ID NO: 355 - RNA, 27 nt, Artificial Sequence
  Synthetic Polynucleotide
agucgagucauaaagucaucaaucacc

SEQ ID NO: 356 - RNA, 27 nt, Artificial Sequence
  Synthetic Polynucleotide
cagucgagucauaaagucaucaaucac

SEQ ID NO: 357 - RNA, 27 nt, Artificial Sequence
  Synthetic Polynucleotide
ccagucgagucauaaagucaucaauca

SEQ ID NO: 358 - RNA, 27 nt, Artificial Sequence
  Synthetic Polynucleotide
uccagucgagucauaaagucaucaauc

SEQ ID NO: 359 - RNA, 27 nt, Artificial Sequence
  Synthetic Polynucleotide
gguccagucgagucauaaagucaucaa

SEQ ID NO: 360 - RNA, 27 nt, Artificial Sequence
  Synthetic Polynucleotide
ugguccagucgagucauaaagucauca

SEQ ID NO: 361 - RNA, 27 nt, Artificial Sequence
  Synthetic Polynucleotide
gcugguccagucgagucauaaagucau

SEQ ID NO: 362 - RNA, 27 nt, Artificial Sequence
  Synthetic Polynucleotide
agcugguccagucgagucauaaaguca

SEQ ID NO: 363 - RNA, 27 nt, Artificial Sequence
  Synthetic Polynucleotide
aguagaagaguugagccauguccgaca

SEQ ID NO: 364 - RNA, 27 nt, Artificial Sequence
  Synthetic Polynucleotide
guaguagaagaguugagccauguccga

SEQ ID NO: 365 - RNA, 27 nt, Artificial Sequence
  Synthetic Polynucleotide
aguaguagaagaguugagccauguccg

SEQ ID NO: 366 - RNA, 27 nt, Artificial Sequence
  Synthetic Polynucleotide
aaggcaaaguaguagaagaguugagcc

SEQ ID NO: 367 - RNA, 27 nt, Artificial Sequence
  Synthetic Polynucleotide
uccaaggcaaaguaguagaagaguuga

SEQ ID NO: 368 - RNA, 27 nt, Artificial Sequence
  Synthetic Polynucleotide
uuccaaggcaaaguaguagaagaguug

SEQ ID NO: 369 - RNA, 27 nt, Artificial Sequence
  Synthetic Polynucleotide
cuuccaaggcaaaguaguagaagaguu

SEQ ID NO: 370 - RNA, 27 nt, Artificial Sequence
  Synthetic Polynucleotide
uagcuuccaaggcaaaguaguagaaga

SEQ ID NO: 371 - RNA, 27 nt, Artificial Sequence
  Synthetic Polynucleotide
auagcuuccaaggcaaaguaguagaag

SEQ ID NO: 372 - RNA, 27 nt, Artificial Sequence
  Synthetic Polynucleotide
aauagcuuccaaggcaaaguaguagaa

SEQ ID NO: 373 - RNA, 27 nt, Artificial Sequence
  Synthetic Polynucleotide
aaauagcuuccaaggcaaaguaguaga

SEQ ID NO: 374 - RNA, 27 nt, Artificial Sequence
  Synthetic Polynucleotide
caaauagcuuccaaggcaaaguaguag

SEQ ID NO: 375 - RNA, 27 nt, Artificial Sequence
  Synthetic Polynucleotide
gcaaauagcuuccaaggcaaaguagua

SEQ ID NO: 376 - RNA, 27 nt, Artificial Sequence
  Synthetic Polynucleotide
agcaaauagcuuccaaggcaaaguagu

SEQ ID NO: 377 - RNA, 27 nt, Artificial Sequence
  Synthetic Polynucleotide
uagcaaauagcuuccaaggcaaaguag

SEQ ID NO: 378 - RNA, 27 nt, Artificial Sequence
  Synthetic Polynucleotide
guagcaaauagcuuccaaggcaaagua

SEQ ID NO: 379 - RNA, 27 nt, Artificial Sequence
  Synthetic Polynucleotide
uguagcaaauagcuuccaaggcaaagu

SEQ ID NO: 380 - RNA, 27 nt, Artificial Sequence
  Synthetic Polynucleotide
auguagcaaauagcuuccaaggcaaag

SEQ ID NO: 381 - RNA, 27 nt, Artificial Sequence
  Synthetic Polynucleotide
gauguagcaaauagcuuccaaggcaaa

SEQ ID NO: 382 - RNA, 27 nt, Artificial Sequence
  Synthetic Polynucleotide
ggauguagcaaauagcuuccaaggcaa

SEQ ID NO: 383 - RNA, 27 nt, Artificial Sequence
  Synthetic Polynucleotide
aggauguagcaaauagcuuccaaggca

SEQ ID NO: 384 - RNA, 27 nt, Artificial Sequence
  Synthetic Polynucleotide
caggauguagcaaauagcuuccaaggc

SEQ ID NO: 385 - RNA, 27 nt, Artificial Sequence
  Synthetic Polynucleotide
acaggauguagcaaauagcuuccaagg

SEQ ID NO: 386 - RNA, 27 nt, Artificial Sequence
  Synthetic Polynucleotide
aacaggauguagcaaauagcuuccaag

SEQ ID NO: 387 - RNA, 27 nt, Artificial Sequence
  Synthetic Polynucleotide
gaacaggauguagcaaauagcuuccaa

SEQ ID NO: 388 - RNA, 27 nt, Artificial Sequence
  Synthetic Polynucleotide
cgaacaggauguagcaaauagcuucca

SEQ ID NO: 389 - RNA, 27 nt, Artificial Sequence
  Synthetic Polynucleotide
ucgaacaggauguagcaaauagcuucc

SEQ ID NO: 390 - RNA, 27 nt, Artificial Sequence
  Synthetic Polynucleotide
cucgaacaggauguagcaaauagcuuc

SEQ ID NO: 391 - RNA, 27 nt, Artificial Sequence
  Synthetic Polynucleotide
ucucgaacaggauguagcaaauagcuu

SEQ ID NO: 392 - RNA, 27 nt, Artificial Sequence
  Synthetic Polynucleotide
uucucgaacaggauguagcaaauagcu

SEQ ID NO: 393 - RNA, 27 nt, Artificial Sequence
  Synthetic Polynucleotide
uuucucgaacaggauguagcaaauagc

SEQ ID NO: 394 - RNA, 27 nt, Artificial Sequence
  Synthetic Polynucleotide
guuucucgaacaggauguagcaaauag

SEQ ID NO: 395 - RNA, 27 nt, Artificial Sequence
  Synthetic Polynucleotide
cguuucucgaacaggauguagcaaaua

SEQ ID NO: 396 - RNA, 27 nt, Artificial Sequence
  Synthetic Polynucleotide
gcguuucucgaacaggauguagcaaau

SEQ ID NO: 397 - RNA, 27 nt, Artificial Sequence
  Synthetic Polynucleotide
ugcguuucucgaacaggauguagcaaa

SEQ ID NO: 398 - RNA, 27 nt, Artificial Sequence
  Synthetic Polynucleotide
augcguuucucgaacaggauguagcaa

SEQ ID NO: 399 - RNA, 27 nt, Artificial Sequence
  Synthetic Polynucleotide
aaugcguuucucgaacaggauguagca

SEQ ID NO: 400 - RNA, 27 nt, Artificial Sequence
  Synthetic Polynucleotide
ggaacauuaacccgauggaucugacga

SEQ ID NO: 401 - RNA, 27 nt, Artificial Sequence
  Synthetic Polynucleotide
uggaacauuaacccgauggaucugacg

SEQ ID NO: 402 - RNA, 27 nt, Artificial Sequence
  Synthetic Polynucleotide
cuggaacauuaacccgauggaucugac

SEQ ID NO: 403 - RNA, 27 nt, Artificial Sequence
  Synthetic Polynucleotide
ucuggaacauuaacccgauggaucuga

SEQ ID NO: 404 - RNA, 27 nt, Artificial Sequence
  Synthetic Polynucleotide
uucuggaacauuaacccgauggaucug

SEQ ID NO: 405 - RNA, 27 nt, Artificial Sequence
  Synthetic Polynucleotide
guucuggaacauuaacccgauggaucu

SEQ ID NO: 406 - RNA, 27 nt, Artificial Sequence
  Synthetic Polynucleotide
aguucuggaacauuaacccgauggauc

SEQ ID NO: 407 - RNA, 27 nt, Artificial Sequence
  Synthetic Polynucleotide
gaguucuggaacauuaacccgauggau

SEQ ID NO: 408 - RNA, 27 nt, Artificial Sequence
  Synthetic Polynucleotide
ugaguucuggaacauuaacccgaugga

SEQ ID NO: 409 - RNA, 27 nt, Artificial Sequence
  Synthetic Polynucleotide
gugaguucuggaacauuaacccgaugg

SEQ ID NO: 410 - RNA, 27 nt, Artificial Sequence
  Synthetic Polynucleotide
agugaguucuggaacauuaacccgaug

SEQ ID NO: 411 - RNA, 27 nt, Artificial Sequence
  Synthetic Polynucleotide
gagugaguucuggaacauuaacccgau

SEQ ID NO: 412 - RNA, 27 nt, Artificial Sequence
  Synthetic Polynucleotide
agagugaguucuggaacauuaacccga

SEQ ID NO: 413 - RNA, 27 nt, Artificial Sequence
  Synthetic Polynucleotide
uagagugaguucuggaacauuaacccg

SEQ ID NO: 414 - RNA, 27 nt, Artificial Sequence
  Synthetic Polynucleotide
auagagugaguucuggaacauuaaccc

SEQ ID NO: 415 - RNA, 27 nt, Artificial Sequence
  Synthetic Polynucleotide
cauagagugaguucuggaacauuaacc

SEQ ID NO: 416 - RNA, 27 nt, Artificial Sequence
  Synthetic Polynucleotide
gcauagagugaguucuggaacauuaac

SEQ ID NO: 417 - RNA, 27 nt, Artificial Sequence
  Synthetic Polynucleotide
ggcauagagugaguucuggaacauuaa

SEQ ID NO: 418 - RNA, 27 nt, Artificial Sequence
  Synthetic Polynucleotide
uggcauagagugaguucuggaacauua

SEQ ID NO: 419 - RNA, 27 nt, Artificial Sequence
  Synthetic Polynucleotide
guggcauagagugaguucuggaacauu

SEQ ID NO: 420 - RNA, 27 nt, Artificial Sequence
  Synthetic Polynucleotide
gguggcauagagugaguucuggaacau

SEQ ID NO: 421 - RNA, 27 nt, Artificial Sequence
  Synthetic Polynucleotide
gaagguggcauagagugaguucuggaa

SEQ ID NO: 422 - RNA, 27 nt, Artificial Sequence
  Synthetic Polynucleotide
ggaagguggcauagagugaguucugga

SEQ ID NO: 423 - RNA, 27 nt, Artificial Sequence
  Synthetic Polynucleotide
uuccaaccauccagguaucgcuuccag

SEQ ID NO: 424 - RNA, 27 nt, Artificial Sequence
  Synthetic Polynucleotide
auuccaaccauccagguaucgcuucca

SEQ ID NO: 425 - RNA, 27 nt, Artificial Sequence
  Synthetic Polynucleotide
cauuccaaccauccagguaucgcuucc

SEQ ID NO: 426 - RNA, 27 nt, Artificial Sequence
  Synthetic Polynucleotide
gcauuccaaccauccagguaucgcuuc

SEQ ID NO: 427 - RNA, 27 nt, Artificial Sequence
  Synthetic Polynucleotide
ggcauuccaaccauccagguaucgcuu

SEQ ID NO: 428 - RNA, 27 nt, Artificial Sequence
  Synthetic Polynucleotide
uggcauuccaaccauccagguaucgcu

SEQ ID NO: 429 - RNA, 27 nt, Artificial Sequence
  Synthetic Polynucleotide
auggcauuccaaccauccagguaucgc

SEQ ID NO: 430 - RNA, 27 nt, Artificial Sequence
  Synthetic Polynucleotide
gauggcauuccaaccauccagguaucg

SEQ ID NO: 431 - RNA, 27 nt, Artificial Sequence
  Synthetic Polynucleotide
aagauggcauuccaaccauccagguau

SEQ ID NO: 432 - RNA, 27 nt, Artificial Sequence
  Synthetic Polynucleotide
aaagauggcauuccaaccauccaggua

SEQ ID NO: 433 - RNA, 27 nt, Artificial Sequence
  Synthetic Polynucleotide
aaaagauggcauuccaaccauccaggu

SEQ ID NO: 434 - RNA, 27 nt, Artificial Sequence
  Synthetic Polynucleotide
gaaaagauggcauuccaaccauccagg

SEQ ID NO: 435 - RNA, 27 nt, Artificial Sequence
  Synthetic Polynucleotide
ggaaaagauggcauuccaaccauccag

SEQ ID NO: 436 - RNA, 27 nt, Artificial Sequence
  Synthetic Polynucleotide
aggaaaagauggcauuccaaccaucca

SEQ ID NO: 437 - RNA, 27 nt, Artificial Sequence
  Synthetic Polynucleotide
aaggaaaagauggcauuccaaccaucc

SEQ ID NO: 438 - RNA, 27 nt, Artificial Sequence
  Synthetic Polynucleotide
aaaggaaaagauggcauuccaaccauc

SEQ ID NO: 439 - RNA, 27 nt, Artificial Sequence
  Synthetic Polynucleotide
caaaggaaaagauggcauuccaaccau

SEQ ID NO: 440 - RNA, 27 nt, Artificial Sequence
  Synthetic Polynucleotide
ccaaaggaaaagauggcauuccaacca

SEQ ID NO: 441 - RNA, 27 nt, Artificial Sequence
  Synthetic Polynucleotide
uucccaaaggaaaagauggcauuccaa

SEQ ID NO: 442 - RNA, 27 nt, Artificial Sequence
  Synthetic Polynucleotide
ucaucaaucagcuucuucccaaaggaa

SEQ ID NO: 443 - RNA, 27 nt, Artificial Sequence
  Synthetic Polynucleotide
gcuucucaucaaucagcuucuucccaa

SEQ ID NO: 444 - RNA, 27 nt, Artificial Sequence
  Synthetic Polynucleotide
agcuucucaucaaucagcuucuuccca

SEQ ID NO: 445 - RNA, 27 nt, Artificial Sequence
  Synthetic Polynucleotide
gagcuucucaucaaucagcuucuuccc

SEQ ID NO: 446 - RNA, 27 nt, Artificial Sequence
  Synthetic Polynucleotide
cgagcuucucaucaaucagcuucuucc

SEQ ID NO: 447 - RNA, 27 nt, Artificial Sequence
  Synthetic Polynucleotide
ucgagcuucucaucaaucagcuucuuc

SEQ ID NO: 448 - RNA, 27 nt, Artificial Sequence
  Synthetic Polynucleotide
uucgagcuucucaucaaucagcuucuu

SEQ ID NO: 449 - RNA, 27 nt, Artificial Sequence
  Synthetic Polynucleotide
cuucgagcuucucaucaaucagcuucu

SEQ ID NO: 450 - RNA, 27 nt, Artificial Sequence
  Synthetic Polynucleotide
ucuucgagcuucucaucaaucagcuuc

SEQ ID NO: 451 - RNA, 27 nt, Artificial Sequence
  Synthetic Polynucleotide
aucuucgagcuucucaucaaucagcuu

SEQ ID NO: 452 - RNA, 27 nt, Artificial Sequence
  Synthetic Polynucleotide
uaucuucgagcuucucaucaaucagcu

SEQ ID NO: 453 - RNA, 27 nt, Artificial Sequence
  Synthetic Polynucleotide
auaucuucgagcuucucaucaaucagc

SEQ ID NO: 454 - RNA, 27 nt, Artificial Sequence
  Synthetic Polynucleotide
cauaucuucgagcuucucaucaaucag

SEQ ID NO: 455 - RNA, 27 nt, Artificial Sequence
  Synthetic Polynucleotide
ccauaucuucgagcuucucaucaauca

SEQ ID NO: 456 - RNA, 27 nt, Artificial Sequence
  Synthetic Polynucleotide
uccauaucuucgagcuucucaucaauc

SEQ ID NO: 457 - RNA, 27 nt, Artificial Sequence
  Synthetic Polynucleotide
cuccauaucuucgagcuucucaucaau

SEQ ID NO: 458 - RNA, 27 nt, Artificial Sequence
  Synthetic Polynucleotide
ccuccauaucuucgagcuucucaucaa

SEQ ID NO: 459 - RNA, 27 nt, Artificial Sequence
  Synthetic Polynucleotide
agaggugguacagggcccaugucagcg

SEQ ID NO: 460 - RNA, 27 nt, Artificial Sequence
  Synthetic Polynucleotide
gagaggugguacagggcccaugucagc

SEQ ID NO: 461 - RNA, 27 nt, Artificial Sequence
  Synthetic Polynucleotide
ugagaggugguacagggcccaugucag

SEQ ID NO: 462 - RNA, 27 nt, Artificial Sequence
  Synthetic Polynucleotide
uugagaggugguacagggcccauguca

SEQ ID NO: 463 - RNA, 27 nt, Artificial Sequence
  Synthetic Polynucleotide
uuugagaggugguacagggcccauguc

SEQ ID NO: 464 - RNA, 27 nt, Artificial Sequence
  Synthetic Polynucleotide
ccucgugcaaggccuccuggaucucag

SEQ ID NO: 465 - RNA, 27 nt, Artificial Sequence
  Synthetic Polynucleotide
uccucgugcaaggccuccuggaucuca

SEQ ID NO: 466 - RNA, 27 nt, Artificial Sequence
  Synthetic Polynucleotide
uuccucgugcaaggccuccuggaucuc

SEQ ID NO: 467 - RNA, 27 nt, Artificial Sequence
  Synthetic Polynucleotide
gggcaaaguccuugugcuggggcacuu

SEQ ID NO: 468 - RNA, 27 nt, Artificial Sequence
  Synthetic Polynucleotide
ugggcaaaguccuugugcuggggcacu

SEQ ID NO: 469 - RNA, 27 nt, Artificial Sequence
  Synthetic Polynucleotide
gugggcaaaguccuugugcuggggcac

SEQ ID NO: 470 - RNA, 27 nt, Artificial Sequence
  Synthetic Polynucleotide
ugugggcaaaguccuugugcuggggca

SEQ ID NO: 471 - RNA, 27 nt, Artificial Sequence
  Synthetic Polynucleotide
augugggcaaaguccuugugcuggggc

SEQ ID NO: 472 - RNA, 27 nt, Artificial Sequence
  Synthetic Polynucleotide
caugugggcaaaguccuugugcugggg

SEQ ID NO: 473 - RNA, 27 nt, Artificial Sequence
  Synthetic Polynucleotide
gcaugugggcaaaguccuugugcuggg

SEQ ID NO: 474 - RNA, 27 nt, Artificial Sequence
  Synthetic Polynucleotide
ggcaugugggcaaaguccuugugcugg

SEQ ID NO: 475 - RNA, 27 nt, Artificial Sequence
  Synthetic Polynucleotide
cggcaugugggcaaaguccuugugcug

SEQ ID NO: 476 - RNA, 27 nt, Artificial Sequence
  Synthetic Polynucleotide
acggcaugugggcaaaguccuugugcu

SEQ ID NO: 477 - RNA, 27 nt, Artificial Sequence
  Synthetic Polynucleotide
aacggcaugugggcaaaguccuugugc

SEQ ID NO: 478 - RNA, 27 nt, Artificial Sequence
  Synthetic Polynucleotide
caacggcaugugggcaaaguccuugug

SEQ ID NO: 479 - RNA, 27 nt, Artificial Sequence
  Synthetic Polynucleotide
gcaacggcaugugggcaaaguccuugu

SEQ ID NO: 480 - RNA, 27 nt, Artificial Sequence
  Synthetic Polynucleotide
agcaacggcaugugggcaaaguccuug

SEQ ID NO: 481 - RNA, 27 nt, Artificial Sequence
  Synthetic Polynucleotide
gagucuccuuaagcacagcuuugagca

SEQ ID NO: 482 - RNA, 27 nt, Artificial Sequence
  Synthetic Polynucleotide
cagagucuccuuaagcacagcuuugag

SEQ ID NO: 483 - RNA, 27 nt, Artificial Sequence
  Synthetic Polynucleotide
uuucuaugauccgggaguuugugggga

SEQ ID NO: 484 - RNA, 27 nt, Artificial Sequence
  Synthetic Polynucleotide
uuuucuaugauccgggaguuugugggg

SEQ ID NO: 485 - RNA, 27 nt, Artificial Sequence
  Synthetic Polynucleotide
ccuuuucuaugauccgggaguuugugg

SEQ ID NO: 486 - RNA, 27 nt, Artificial Sequence
  Synthetic Polynucleotide
uccuuuucuaugauccgggaguuugug

SEQ ID NO: 487 - RNA, 27 nt, Artificial Sequence
  Synthetic Polynucleotide
caauuuccuuuucuaugauccgggagu

SEQ ID NO: 488 - RNA, 27 nt, Artificial Sequence
  Synthetic Polynucleotide
ucaauuuccuuuucuaugauccgggag

SEQ ID NO: 489 - RNA, 27 nt, Artificial Sequence
  Synthetic Polynucleotide
uucaauuuccuuuucuaugauccggga

SEQ ID NO: 490 - RNA, 27 nt, Artificial Sequence
  Synthetic Polynucleotide
cuucaauuuccuuuucuaugauccggg

SEQ ID NO: 491 - RNA, 27 nt, Artificial Sequence
  Synthetic Polynucleotide
acuucaauuuccuuuucuaugauccgg

SEQ ID NO: 492 - RNA, 27 nt, Artificial Sequence
  Synthetic Polynucleotide
aacuucaauuuccuuuucuaugauccg

SEQ ID NO: 493 - RNA, 27 nt, Artificial Sequence
  Synthetic Polynucleotide
caacuucaauuuccuuuucuaugaucc

SEQ ID NO: 494 - RNA, 27 nt, Artificial Sequence
  Synthetic Polynucleotide
ucaacuucaauuuccuuuucuaugauc

SEQ ID NO: 495 - RNA, 27 nt, Artificial Sequence
  Synthetic Polynucleotide
aucaacuucaauuuccuuuucuaugau

SEQ ID NO: 496 - RNA, 27 nt, Artificial Sequence
  Synthetic Polynucleotide
caucaacuucaauuuccuuuucuauga

SEQ ID NO: 497 - RNA, 27 nt, Artificial Sequence
  Synthetic Polynucleotide
ccaucaacuucaauuuccuuuucuaug

SEQ ID NO: 498 - RNA, 27 nt, Artificial Sequence
  Synthetic Polynucleotide
gccaucaacuucaauuuccuuuucuau

SEQ ID NO: 499 - RNA, 27 nt, Artificial Sequence
  Synthetic Polynucleotide
agccaucaacuucaauuuccuuuucua

SEQ ID NO: 500 - RNA, 27 nt, Artificial Sequence
  Synthetic Polynucleotide
aagccaucaacuucaauuuccuuuucu

SEQ ID NO: 501 - RNA, 27 nt, Artificial Sequence
  Synthetic Polynucleotide
gaagccaucaacuucaauuuccuuuuc

SEQ ID NO: 502 - RNA, 27 nt, Artificial Sequence
  Synthetic Polynucleotide
gaggaagccaucaacuucaauuuccuu

SEQ ID NO: 503 - RNA, 27 nt, Artificial Sequence
  Synthetic Polynucleotide
agaggaagccaucaacuucaauuuccu

SEQ ID NO: 504 - RNA, 27 nt, Artificial Sequence
  Synthetic Polynucleotide
aagaggaagccaucaacuucaauuucc

SEQ ID NO: 505 - RNA, 27 nt, Artificial Sequence
  Synthetic Polynucleotide
ccuuguacuucuggaucagccuugcga

SEQ ID NO: 506 - RNA, 27 nt, Artificial Sequence
  Synthetic Polynucleotide
accuuguacuucuggaucagccuugcg

SEQ ID NO: 507 - RNA, 27 nt, Artificial Sequence
  Synthetic Polynucleotide
caccuuguacuucuggaucagccuugc

SEQ ID NO: 508 - RNA, 27 nt, Artificial Sequence
  Synthetic Polynucleotide
ccaccuuguacuucuggaucagccuug

SEQ ID NO: 509 - RNA, 27 nt, Artificial Sequence
  Synthetic Polynucleotide
ucuuauugggaaccaggacaaugcggg

SEQ ID NO: 510 - RNA, 27 nt, Artificial Sequence
  Synthetic Polynucleotide
uucuuauugggaaccaggacaaugcgg

SEQ ID NO: 511 - RNA, 27 nt, Artificial Sequence
  Synthetic Polynucleotide
uuucuuauugggaaccaggacaaugcg

SEQ ID NO: 512 - RNA, 27 nt, Artificial Sequence
  Synthetic Polynucleotide
cuuucuuauugggaaccaggacaaugc

SEQ ID NO: 513 - RNA, 27 nt, Artificial Sequence
  Synthetic Polynucleotide
acuuucuuauugggaaccaggacaaug

SEQ ID NO: 514 - RNA, 27 nt, Artificial Sequence
  Synthetic Polynucleotide
cacuuucuuauugggaaccaggacaau

SEQ ID NO: 515 - RNA, 27 nt, Artificial Sequence
  Synthetic Polynucleotide
ccacuuucuuauugggaaccaggacaa

SEQ ID NO: 516 - RNA, 27 nt, Artificial Sequence
  Synthetic Polynucleotide
gauagaaguggcaaaagcucagggugu

SEQ ID NO: 517 - RNA, 27 nt, Artificial Sequence
  Synthetic Polynucleotide
ugauagaaguggcaaaagcucagggug

SEQ ID NO: 518 - RNA, 27 nt, Artificial Sequence
  Synthetic Polynucleotide
augauagaaguggcaaaagcucagggu

SEQ ID NO: 519 - RNA, 27 nt, Artificial Sequence
  Synthetic Polynucleotide
aaugauagaaguggcaaaagcucaggg

SEQ ID NO: 520 - RNA, 27 nt, Artificial Sequence
  Synthetic Polynucleotide
aaaugauagaaguggcaaaagcucagg

SEQ ID NO: 521 - RNA, 27 nt, Artificial Sequence
  Synthetic Polynucleotide
aaaaugauagaaguggcaaaagcucag

SEQ ID NO: 522 - RNA, 27 nt, Artificial Sequence
  Synthetic Polynucleotide
aaaaaugauagaaguggcaaaagcuca

SEQ ID NO: 523 - RNA, 27 nt, Artificial Sequence
  Synthetic Polynucleotide
caaaaaugauagaaguggcaaaagcuc

SEQ ID NO: 524 - RNA, 27 nt, Artificial Sequence
  Synthetic Polynucleotide
ucaaaaaugauagaaguggcaaaagcu

SEQ ID NO: 525 - RNA, 27 nt, Artificial Sequence
  Synthetic Polynucleotide
cucaaaaaugauagaaguggcaaaagc

SEQ ID NO: 526 - RNA, 27 nt, Artificial Sequence
  Synthetic Polynucleotide
gcucaaaaaugauagaaguggcaaaag

SEQ ID NO: 527 - RNA, 27 nt, Artificial Sequence
  Synthetic Polynucleotide
ugcucaaaaaugauagaaguggcaaaa

SEQ ID NO: 528 - RNA, 27 nt, Artificial Sequence
  Synthetic Polynucleotide
uugcucaaaaaugauagaaguggcaaa

SEQ ID NO: 529 - RNA, 27 nt, Artificial Sequence
  Synthetic Polynucleotide
guugcucaaaaaugauagaaguggcaa

SEQ ID NO: 530 - RNA, 27 nt, Artificial Sequence
  Synthetic Polynucleotide
aguugcucaaaaaugauagaaguggca

SEQ ID NO: 531 - RNA, 27 nt, Artificial Sequence
  Synthetic Polynucleotide
gaguugcucaaaaaugauagaaguggc

SEQ ID NO: 532 - RNA, 27 nt, Artificial Sequence
  Synthetic Polynucleotide
ggaguugcucaaaaaugauagaagugg

SEQ ID NO: 533 - RNA, 27 nt, Artificial Sequence
  Synthetic Polynucleotide
gggaguugcucaaaaaugauagaagug

SEQ ID NO: 534 - RNA, 27 nt, Artificial Sequence
  Synthetic Polynucleotide
agggaguugcucaaaaaugauagaagu

SEQ ID NO: 535 - RNA, 27 nt, Artificial Sequence
  Synthetic Polynucleotide
gagggaguugcucaaaaaugauagaag

SEQ ID NO: 536 - RNA, 27 nt, Artificial Sequence
  Synthetic Polynucleotide
agagggaguugcucaaaaaugauagaa

SEQ ID NO: 537 - RNA, 27 nt, Artificial Sequence
  Synthetic Polynucleotide
ugagagggaguugcucaaaaaugauag

SEQ ID NO: 538 - RNA, 27 nt, Artificial Sequence
  Synthetic Polynucleotide
cugagagggaguugcucaaaaaugaua

SEQ ID NO: 539 - RNA, 27 nt, Artificial Sequence
  Synthetic Polynucleotide
ccuuuuagcugagagggaguugcucaa

SEQ ID NO: 540 - RNA, 27 nt, Artificial Sequence
  Synthetic Polynucleotide
uauucuacccaaggacagcaaugcgau

SEQ ID NO: 541 - RNA, 27 nt, Artificial Sequence
  Synthetic Polynucleotide
auauucuacccaaggacagcaaugcga

SEQ ID NO: 542 - RNA, 27 nt, Artificial Sequence
  Synthetic Polynucleotide
uauauucuacccaaggacagcaaugcg

SEQ ID NO: 543 - RNA, 27 nt, Artificial Sequence
  Synthetic Polynucleotide
uuauauucuacccaaggacagcaaugc

SEQ ID NO: 544 - RNA, 27 nt, Artificial Sequence
  Synthetic Polynucleotide
uuuauauucuacccaaggacagcaaug

SEQ ID NO: 545 - RNA, 27 nt, Artificial Sequence
  Synthetic Polynucleotide
uuuuauauucuacccaaggacagcaau

SEQ ID NO: 546 - RNA, 27 nt, Artificial Sequence
  Synthetic Polynucleotide
auuuuauauucuacccaaggacagcaa

SEQ ID NO: 547 - RNA, 27 nt, Artificial Sequence
  Synthetic Polynucleotide
uauuuuauauucuacccaaggacagca

SEQ ID NO: 548 - RNA, 27 nt, Artificial Sequence
  Synthetic Polynucleotide
uuauuuuauauucuacccaaggacagc

SEQ ID NO: 549 - RNA, 27 nt, Artificial Sequence
  Synthetic Polynucleotide
uuuauuuuauauucuacccaaggacag

SEQ ID NO: 550 - RNA, 27 nt, Artificial Sequence
  Synthetic Polynucleotide
cuuuauuuuauauucuacccaaggaca

SEQ ID NO: 551 - RNA, 27 nt, Artificial Sequence
  Synthetic Polynucleotide
ccuuuauuuuauauucuacccaaggac

SEQ ID NO: 552 - RNA, 27 nt, Artificial Sequence
  Synthetic Polynucleotide
cccuuuauuuuauauucuacccaagga

SEQ ID NO: 553 - RNA, 27 nt, Artificial Sequence
  Synthetic Polynucleotide
ucccuuuauuuuauauucuacccaagg

SEQ ID NO: 554 - RNA, 27 nt, Artificial Sequence
  Synthetic Polynucleotide
gucccuuuauuuuauauucuacccaag

SEQ ID NO: 555 - RNA, 27 nt, Artificial Sequence
  Synthetic Polynucleotide
agucccuuuauuuuauauucuacccaa

SEQ ID NO: 556 - RNA, 27 nt, Artificial Sequence
  Synthetic Polynucleotide
aaagucccuuuauuuuauauucuaccc

SEQ ID NO: 557 - RNA, 27 nt, Artificial Sequence
  Synthetic Polynucleotide
aaaagucccuuuauuuuauauucuacc

SEQ ID NO: 558 - RNA, 27 nt, Artificial Sequence
  Synthetic Polynucleotide
uaaaagucccuuuauuuuauauucuac

SEQ ID NO: 559 - RNA, 27 nt, Artificial Sequence
  Synthetic Polynucleotide
auaaaagucccuuuauuuuauauucua

SEQ ID NO: 560 - RNA, 27 nt, Artificial Sequence
  Synthetic Polynucleotide
aauaaaagucccuuuauuuuauauucu

SEQ ID NO: 561 - RNA, 27 nt, Artificial Sequence
  Synthetic Polynucleotide
aaauaaaagucccuuuauuuuauauuc

SEQ ID NO: 562 - RNA, 27 nt, Artificial Sequence
  Synthetic Polynucleotide
gaaauaaaagucccuuuauuuuauauu

SEQ ID NO: 563 - RNA, 27 nt, Artificial Sequence
  Synthetic Polynucleotide
agaaauaaaagucccuuuauuuuauau

SEQ ID NO: 564 - RNA, 27 nt, Artificial Sequence
  Synthetic Polynucleotide
aagaaauaaaagucccuuuauuuuaua

SEQ ID NO: 565 - RNA, 27 nt, Artificial Sequence
  Synthetic Polynucleotide
uaagaaauaaaagucccuuuauuuuau

SEQ ID NO: 566 - RNA, 27 nt, Artificial Sequence
  Synthetic Polynucleotide
auaagaaauaaaagucccuuuauuuua

SEQ ID NO: 567 - RNA, 27 nt, Artificial Sequence
  Synthetic Polynucleotide
aauaagaaauaaaagucccuuuauuuu

SEQ ID NO: 568 - RNA, 27 nt, Artificial Sequence
  Synthetic Polynucleotide
caauaagaaauaaaagucccuuuauuu

SEQ ID NO: 569 - RNA, 27 nt, Artificial Sequence
  Synthetic Polynucleotide
ccaauaagaaauaaaagucccuuuauu

SEQ ID NO: 570 - RNA, 27 nt, Artificial Sequence
  Synthetic Polynucleotide
uccaauaagaaauaaaagucccuuuau

SEQ ID NO: 571 - RNA, 27 nt, Artificial Sequence
  Synthetic Polynucleotide
uuccaauaagaaauaaaagucccuuua

SEQ ID NO: 572 - RNA, 27 nt, Artificial Sequence
  Synthetic Polynucleotide
uuuccaauaagaaauaaaagucccuuu

SEQ ID NO: 573 - RNA, 27 nt, Artificial Sequence
  Synthetic Polynucleotide
uuuuccaauaagaaauaaaagucccuu

SEQ ID NO: 574 - RNA, 27 nt, Artificial Sequence
  Synthetic Polynucleotide
uuuuuccaauaagaaauaaaagucccu

SEQ ID NO: 575 - RNA, 27 nt, Artificial Sequence
  Synthetic Polynucleotide
uuuuuuccaauaagaaauaaaaguccc

SEQ ID NO: 576 - RNA, 27 nt, Artificial Sequence
  Synthetic Polynucleotide
uuuuuuuccaauaagaaauaaaagucc

SEQ ID NO: 577 - RNA, 36 nt, Artificial Sequence
  Synthetic Polynucleotide
ugcuacauccuguucgagaagcagccgaaaggcugc

SEQ ID NO: 578 - RNA, 36 nt, Artificial Sequence
  Synthetic Polynucleotide
cagaacucacucuaugccacgcagccgaaaggcugc

SEQ ID NO: 579 - RNA, 22 nt, Artificial Sequence
  Synthetic Polynucleotide
uucucgaacaggauguagcaaa

SEQ ID NO: 580 - RNA, 22 nt, Artificial Sequence
  Synthetic Polynucleotide
guggcauagagugaguucugga

SEQ ID NO: 581 - RNA, 36 nt, Artificial Sequence
  Synthetic Polynucleotide
cggaugcuuucaaugagguagcagccgaaaggcugc

SEQ ID NO: 582 - RNA, 36 nt, Artificial Sequence
  Synthetic Polynucleotide
augaggugauugaugacuuugcagccgaaaggcugc

SEQ ID NO: 583 - RNA, 36 nt, Artificial Sequence
  Synthetic Polynucleotide
aggugauugaugacuuuauggcagccgaaaggcugc

SEQ ID NO: 584 - RNA, 36 nt, Artificial Sequence
  Synthetic Polynucleotide
gugauugaugacuuuaugaagcagccgaaaggcugc

SEQ ID NO: 585 - RNA, 36 nt, Artificial Sequence
  Synthetic Polynucleotide
auugaugacuuuaugacucagcagccgaaaggcugc

SEQ ID NO: 586 - RNA, 36 nt, Artificial Sequence
  Synthetic Polynucleotide
ucuacuacuuugccuuggaagcagccgaaaggcugc

SEQ ID NO: 587 - RNA, 36 nt, Artificial Sequence
  Synthetic Polynucleotide
cuacuuugccuuggaagcuagcagccgaaaggcugc

SEQ ID NO: 588 - RNA, 36 nt, Artificial Sequence
  Synthetic Polynucleotide
auuugcuacauccuguucgagcagccgaaaggcugc

SEQ ID NO: 589 - RNA, 36 nt, Artificial Sequence
  Synthetic Polynucleotide
uugcuacauccuguucgagagcagccgaaaggcugc

SEQ ID NO: 590 - RNA, 36 nt, Artificial Sequence
  Synthetic Polynucleotide
uguuccagaacucacucuaugcagccgaaaggcugc

SEQ ID NO: 591 - RNA, 36 nt, Artificial Sequence
  Synthetic Polynucleotide
agaagcugauugaugagaaagcagccgaaaggcugc

SEQ ID NO: 592 - RNA, 36 nt, Artificial Sequence
  Synthetic Polynucleotide
aggacuuugcccacaugccagcagccgaaaggcugc

SEQ ID NO: 593 - RNA, 36 nt, Artificial Sequence
  Synthetic Polynucleotide
ucccggaucauagaaaaggagcagccgaaaggcugc

SEQ ID NO: 594 - RNA, 36 nt, Artificial Sequence
  Synthetic Polynucleotide
cggaucauagaaaaggaaaugcagccgaaaggcugc

SEQ ID NO: 595 - RNA, 36 nt, Artificial Sequence
  Synthetic Polynucleotide
gaaauugaaguugauggcuugcagccgaaaggcugc

SEQ ID NO: 596 - RNA, 36 nt, Artificial Sequence
  Synthetic Polynucleotide
aaggcugauccagaaguacagcagccgaaaggcugc

SEQ ID NO: 597 - RNA, 36 nt, Artificial Sequence
  Synthetic Polynucleotide
guccuuggguagaauauaaagcagccgaaaggcugc

SEQ ID NO: 598 - RNA, 22 nt, Artificial Sequence
  Synthetic Polynucleotide
uaccucauugaaagcauccggg

SEQ ID NO: 599 - RNA, 22 nt, Artificial Sequence
  Synthetic Polynucleotide
aaagucaucaaucaccucaugg

SEQ ID NO: 600 - RNA, 22 nt, Artificial Sequence
  Synthetic Polynucleotide
  misc_feature (1)..(1): n is c or u
nauaaagucaucaaucaccugg

SEQ ID NO: 601 - RNA, 22 nt, Artificial Sequence
  Synthetic Polynucleotide
uucauaaagucaucaaucacgg

SEQ ID NO: 602 - RNA, 22 nt, Artificial Sequence
  Synthetic Polynucleotide
ugagucauaaagucaucaaugg

SEQ ID NO: 603 - RNA, 22 nt, Artificial Sequence
  Synthetic Polynucleotide
uuccaaggcaaaguaguagagg

SEQ ID NO: 604 - RNA, 22 nt, Artificial Sequence
  Synthetic Polynucleotide
uagcuuccaaggcaaaguaggg

SEQ ID NO: 605 - RNA, 22 nt, Artificial Sequence
  Synthetic Polynucleotide
ucgaacaggauguagcaaaugg

SEQ ID NO: 606 - RNA, 22 nt, Artificial Sequence
  Synthetic Polynucleotide
ucucgaacaggauguagcaagg

SEQ ID NO: 607 - RNA, 22 nt, Artificial Sequence
  Synthetic Polynucleotide
auagagugaguucuggaacagg

SEQ ID NO: 608 - RNA, 22 nt, Artificial Sequence
  Synthetic Polynucleotide
  misc_feature (1)..(1): n is c or u
nuucucaucaaucagcuucugg

SEQ ID NO: 609 - RNA, 22 nt, Artificial Sequence
  Synthetic Polynucleotide
uggcaugugggcaaaguccugg

SEQ ID NO: 610 - RNA, 22 nt, Artificial Sequence
  Synthetic Polynucleotide
uccuuuucuaugauccgggagg

SEQ ID NO: 611 - RNA, 22 nt, Artificial Sequence
  Synthetic Polynucleotide
auuuccuuuucuaugauccggg

SEQ ID NO: 612 - RNA, 22 nt, Artificial Sequence
  Synthetic Polynucleotide
aagccaucaacuucaauuucgg

SEQ ID NO: 613 - RNA, 22 nt, Artificial Sequence
  Synthetic Polynucleotide
uguacuucuggaucagccuugg

SEQ ID NO: 614 - RNA, 22 nt, Artificial Sequence
  Synthetic Polynucleotide
uuuauauucuacccaaggacgg

SEQ ID NO: 615 - RNA, 25 nt, Artificial Sequence
  Synthetic Polynucleotide
ccggaacgcuacaauuuuuauucca

SEQ ID NO: 616 - RNA, 25 nt, Artificial Sequence
  Synthetic Polynucleotide
cggaacgcuacaauuuuuauuccag

SEQ ID NO: 617 - DNA, 25 bp, Artificial Sequence
  Synthetic Polynucleotide
acgcuacaauuuuuauuccagcuat

SEQ ID NO: 618 - DNA, 25 bp, Artificial Sequence
  Synthetic Polynucleotide
cgcuacaauuuuuauuccagcuatt

SEQ ID NO: 619 - DNA, 25 bp, Artificial Sequence
  Synthetic Polynucleotide
acaauuuuuauuccagcuauuucta

SEQ ID NO: 620 - RNA, 25 nt, Artificial Sequence
  Synthetic Polynucleotide
caauuuuuauuccagcuauuucuac

SEQ ID NO: 621 - RNA, 25 nt, Artificial Sequence
  Synthetic Polynucleotide
aauuuuuauuccagcuauuucuaca

SEQ ID NO: 622 - RNA, 25 nt, Artificial Sequence
  Synthetic Polynucleotide
auuuuuauuccagcuauuucuacaa

SEQ ID NO: 623 - DNA, 25 bp, Artificial Sequence
  Synthetic Polynucleotide
caggugcugaacaagaccaaguatg

SEQ ID NO: 624 - RNA, 25 nt, Artificial Sequence
  Synthetic Polynucleotide
aacgagguuaucagugacuuuauca

SEQ ID NO: 625 - RNA, 25 nt, Artificial Sequence
  Synthetic Polynucleotide
acgagguuaucagugacuuuaucac

SEQ ID NO: 626 - RNA, 25 nt, Artificial Sequence
  Synthetic Polynucleotide
cgagguuaucagugacuuuaucacc

SEQ ID NO: 627 - DNA, 25 bp, Artificial Sequence
  Synthetic Polynucleotide
ggaagccaucaccuauauccugutt

SEQ ID NO: 628 - DNA, 25 bp, Artificial Sequence
  Synthetic Polynucleotide
gaagccaucaccuauauccuguutg

SEQ ID NO: 629 - RNA, 25 nt, Artificial Sequence
  Synthetic Polynucleotide
aagccaucaccuauauccuguuuga

SEQ ID NO: 630 - RNA, 25 nt, Artificial Sequence
  Synthetic Polynucleotide
gccaucaccuauauccuguuugaga

SEQ ID NO: 631 - RNA, 25 nt, Artificial Sequence
  Synthetic Polynucleotide
ccaucaccuauauccuguuugagaa

SEQ ID NO: 632 - RNA, 25 nt, Artificial Sequence
  Synthetic Polynucleotide
caucaccuauauccuguuugagaaa

SEQ ID NO: 633 - RNA, 25 nt, Artificial Sequence
  Synthetic Polynucleotide
aucaccuauauccuguuugagaaaa

SEQ ID NO: 634 - RNA, 25 nt, Artificial Sequence
  Synthetic Polynucleotide
accuauauccuguuugagaaaagga

SEQ ID NO: 635 - DNA, 25 bp, Artificial Sequence
  Synthetic Polynucleotide
ccuauauccuguuugagaaaaggat

SEQ ID NO: 636 - DNA, 25 bp, Artificial Sequence
  Synthetic Polynucleotide
cuauauccuguuugagaaaaggatt

SEQ ID NO: 637 - RNA, 25 nt, Artificial Sequence
  Synthetic Polynucleotide
agaucuguugcaaucauguuccaga

SEQ ID NO: 638 - RNA, 25 nt, Artificial Sequence
  Synthetic Polynucleotide
gaucuguugcaaucauguuccagaa

SEQ ID NO: 639 - RNA, 25 nt, Artificial Sequence
  Synthetic Polynucleotide
uguugcaaucauguuccagaacuca

SEQ ID NO: 640 - RNA, 25 nt, Artificial Sequence
  Synthetic Polynucleotide
guugcaaucauguuccagaacucag

SEQ ID NO: 641 - DNA, 25 bp, Artificial Sequence
  Synthetic Polynucleotide
caaucauguuccagaacucagucta

SEQ ID NO: 642 - DNA, 25 bp, Artificial Sequence
  Synthetic Polynucleotide
aucauguuccagaacucagucuata

SEQ ID NO: 643 - RNA, 25 nt, Artificial Sequence
  Synthetic Polynucleotide
auguuccagaacucagucuauauca

SEQ ID NO: 644 - RNA, 25 nt, Artificial Sequence
  Synthetic Polynucleotide
uguuccagaacucagucuauaucac

SEQ ID NO: 645 - DNA, 25 bp, Artificial Sequence
  Synthetic Polynucleotide
guuccagaacucagucuauaucact

SEQ ID NO: 646 - DNA, 25 bp, Artificial Sequence
  Synthetic Polynucleotide
uuccagaacucagucuauaucactt

SEQ ID NO: 647 - DNA, 25 bp, Artificial Sequence
  Synthetic Polynucleotide
ccagaacucagucuauaucacuutc

SEQ ID NO: 648 - DNA, 25 bp, Artificial Sequence
  Synthetic Polynucleotide
gaacucagucuauaucacuuucctt

SEQ ID NO: 649 - RNA, 25 nt, Artificial Sequence
  Synthetic Polynucleotide
auaacauuuucuccuuuggaaagaa

SEQ ID NO: 650 - RNA, 25 nt, Artificial Sequence
  Synthetic Polynucleotide
uaacauuuucuccuuuggaaagaag

SEQ ID NO: 651 - RNA, 25 nt, Artificial Sequence
  Synthetic Polynucleotide
aacauuuucuccuuuggaaagaagc

SEQ ID NO: 652 - RNA, 25 nt, Artificial Sequence
  Synthetic Polynucleotide
ggaaagaagcugauugaugaaaaag

SEQ ID NO: 653 - DNA, 25 bp, Artificial Sequence
  Synthetic Polynucleotide
gaaagaagcugauugaugaaaaagt

SEQ ID NO: 654 - RNA, 25 nt, Artificial Sequence
  Synthetic Polynucleotide
agaagcugauugaugaaaaagucca

SEQ ID NO: 655 - DNA, 25 bp, Artificial Sequence
  Synthetic Polynucleotide
cugcugaccaaugaauugcucagta

SEQ ID NO: 656 - DNA, 25 bp, Artificial Sequence
  Synthetic Polynucleotide
gcugaccaaugaauugcucaguact

SEQ ID NO: 657 - DNA, 25 bp, Artificial Sequence
  Synthetic Polynucleotide
cugaccaaugaauugcucaguactc

SEQ ID NO: 658 - RNA, 25 nt, Artificial Sequence
  Synthetic Polynucleotide
gaccaaugaauugcucaguacucag

SEQ ID NO: 659 - RNA, 25 nt, Artificial Sequence
  Synthetic Polynucleotide
accaaugaauugcucaguacucagg

SEQ ID NO: 660 - RNA, 25 nt, Artificial Sequence
  Synthetic Polynucleotide
ccaaugaauugcucaguacucagga

SEQ ID NO: 661 - RNA, 25 nt, Artificial Sequence
  Synthetic Polynucleotide
aaugaauugcucaguacucaggaga

SEQ ID NO: 662 - RNA, 25 nt, Artificial Sequence
  Synthetic Polynucleotide
augaauugcucaguacucaggagac

SEQ ID NO: 663 - RNA, 25 nt, Artificial Sequence
  Synthetic Polynucleotide
ggaucaucacagaaaaggaaacuga

SEQ ID NO: 664 - RNA, 25 nt, Artificial Sequence
  Synthetic Polynucleotide
gaucaucacagaaaaggaaacugaa

SEQ ID NO: 665 - DNA, 25 bp, Artificial Sequence
  Synthetic Polynucleotide
aucacagaaaaggaaacugaaauta

SEQ ID NO: 666 - RNA, 25 nt, Artificial Sequence
  Synthetic Polynucleotide
ucacagaaaaggaaacugaaauuaa

SEQ ID NO: 667 - DNA, 25 bp, Artificial Sequence
  Synthetic Polynucleotide
cacagaaaaggaaacugaaauuaat

SEQ ID NO: 668 - DNA, 25 bp, Artificial Sequence
  Synthetic Polynucleotide
acagaaaaggaaacugaaauuaatg

SEQ ID NO: 669 - DNA, 25 bp, Artificial Sequence
  Synthetic Polynucleotide
gaaaaggaaacugaaauuaauggct

SEQ ID NO: 670 - DNA, 25 bp, Artificial Sequence
  Synthetic Polynucleotide
aaaaggaaacugaaauuaauggctt

SEQ ID NO: 671 - DNA, 25 bp, Artificial Sequence
  Synthetic Polynucleotide
ggaaacugaaauuaauggcuuuctc

SEQ ID NO: 672 - RNA, 25 nt, Artificial Sequence
  Synthetic Polynucleotide
agacagcagagcaccuuauaauaac

SEQ ID NO: 673 - RNA, 25 nt, Artificial Sequence
  Synthetic Polynucleotide
gacagcagagcaccuuauaauaaca

SEQ ID NO: 674 - DNA, 25 bp, Artificial Sequence
  Synthetic Polynucleotide
cagcagagcaccuuauaauaacagt

SEQ ID NO: 675 - DNA, 25 bp, Artificial Sequence
  Synthetic Polynucleotide
agcagagcaccuuauaauaacagtc

SEQ ID NO: 676 - DNA, 25 bp, Artificial Sequence
  Synthetic Polynucleotide
gagcaccuuauaauaacaguccutg

SEQ ID NO: 677 - DNA, 25 bp, Artificial Sequence
  Synthetic Polynucleotide
auaauaacaguccuuggguaugatt

SEQ ID NO: 678 - DNA, 25 bp, Artificial Sequence
  Synthetic Polynucleotide
acaguccuuggguaugauuuaaaat

SEQ ID NO: 679 - DNA, 25 bp, Artificial Sequence
  Synthetic Polynucleotide
caguccuuggguaugauuuaaaata

SEQ ID NO: 680 - RNA, 25 nt, Artificial Sequence
  Synthetic Polynucleotide
aguccuuggguaugauuuaaaauaa

SEQ ID NO: 681 - RNA, 25 nt, Artificial Sequence
  Synthetic Polynucleotide
guccuuggguaugauuuaaaauaaa

SEQ ID NO: 682 - RNA, 25 nt, Artificial Sequence
  Synthetic Polynucleotide
uccuuggguaugauuuaaaauaaaa

SEQ ID NO: 683 - DNA, 25 bp, Artificial Sequence
  Synthetic Polynucleotide
cuuggguaugauuuaaaauaaaatt

SEQ ID NO: 684 - DNA, 25 bp, Artificial Sequence
  Synthetic Polynucleotide
uuggguaugauuuaaaauaaaautt

SEQ ID NO: 685 - DNA, 25 bp, Artificial Sequence
  Synthetic Polynucleotide
uggguaugauuuaaaauaaaauuta

SEQ ID NO: 686 - RNA, 25 nt, Artificial Sequence
  Synthetic Polynucleotide
ggguaugauuuaaaauaaaauuuaa

SEQ ID NO: 687 - RNA, 27 nt, Artificial Sequence
  Synthetic Polynucleotide
uggaauaaaaauuguagcguuccgguc

SEQ ID NO: 688 - RNA, 27 nt, Artificial Sequence
  Synthetic Polynucleotide
cuggaauaaaaauuguagcguuccggu

SEQ ID NO: 689 - RNA, 27 nt, Artificial Sequence
  Synthetic Polynucleotide
auagcuggaauaaaaauuguagcguuc

SEQ ID NO: 690 - RNA, 27 nt, Artificial Sequence
  Synthetic Polynucleotide
aauagcuggaauaaaaauuguagcguu

SEQ ID NO: 691 - RNA, 27 nt, Artificial Sequence
  Synthetic Polynucleotide
uagaaauagcuggaauaaaaauuguag

SEQ ID NO: 692 - RNA, 27 nt, Artificial Sequence
  Synthetic Polynucleotide
guagaaauagcuggaauaaaaauugua

SEQ ID NO: 693 - RNA, 27 nt, Artificial Sequence
  Synthetic Polynucleotide
uguagaaauagcuggaauaaaaauugu

SEQ ID NO: 694 - RNA, 27 nt, Artificial Sequence
  Synthetic Polynucleotide
uuguagaaauagcuggaauaaaaauug

SEQ ID NO: 695 - RNA, 27 nt, Artificial Sequence
  Synthetic Polynucleotide
cauacuuggucuuguucagcaccugga

SEQ ID NO: 696 - RNA, 27 nt, Artificial Sequence
  Synthetic Polynucleotide
ugauaaagucacugauaaccucguuua

SEQ ID NO: 697 - RNA, 27 nt, Artificial Sequence
  Synthetic Polynucleotide
gugauaaagucacugauaaccucguuu

SEQ ID NO: 698 - RNA, 27 nt, Artificial Sequence
  Synthetic Polynucleotide
ggugauaaagucacugauaaccucguu

SEQ ID NO: 699 - RNA, 27 nt, Artificial Sequence
  Synthetic Polynucleotide
aaacaggauauaggugauggcuuccaa

SEQ ID NO: 700 - RNA, 27 nt, Artificial Sequence
  Synthetic Polynucleotide
caaacaggauauaggugauggcuucca

SEQ ID NO: 701 - RNA, 27 nt, Artificial Sequence
  Synthetic Polynucleotide
ucaaacaggauauaggugauggcuucc

SEQ ID NO: 702 - RNA, 27 nt, Artificial Sequence
  Synthetic Polynucleotide
ucucaaacaggauauaggugauggcuu

SEQ ID NO: 703 - RNA, 27 nt, Artificial Sequence
  Synthetic Polynucleotide
uucucaaacaggauauaggugauggcu

SEQ ID NO: 704 - RNA, 27 nt, Artificial Sequence
  Synthetic Polynucleotide
uuucucaaacaggauauaggugauggc

SEQ ID NO: 705 - RNA, 27 nt, Artificial Sequence
  Synthetic Polynucleotide
uuuucucaaacaggauauaggugaugg

SEQ ID NO: 706 - RNA, 27 nt, Artificial Sequence
  Synthetic Polynucleotide
uccuuuucucaaacaggauauagguga

SEQ ID NO: 707 - RNA, 27 nt, Artificial Sequence
  Synthetic Polynucleotide
auccuuuucucaaacaggauauaggug

SEQ ID NO: 708 - RNA, 27 nt, Artificial Sequence
  Synthetic Polynucleotide
aauccuuuucucaaacaggauauaggu

SEQ ID NO: 709 - RNA, 27 nt, Artificial Sequence
  Synthetic Polynucleotide
ucuggaacaugauugcaacagaucuga

SEQ ID NO: 710 - RNA, 27 nt, Artificial Sequence
  Synthetic Polynucleotide
uucuggaacaugauugcaacagaucug

SEQ ID NO: 711 - RNA, 27 nt, Artificial Sequence
  Synthetic Polynucleotide
ugaguucuggaacaugauugcaacaga

SEQ ID NO: 712 - RNA, 27 nt, Artificial Sequence
  Synthetic Polynucleotide
cugaguucuggaacaugauugcaacag

SEQ ID NO: 713 - RNA, 27 nt, Artificial Sequence
  Synthetic Polynucleotide
uagacugaguucuggaacaugauugca

SEQ ID NO: 714 - RNA, 27 nt, Artificial Sequence
  Synthetic Polynucleotide
uauagacugaguucuggaacaugauug

SEQ ID NO: 715 - RNA, 27 nt, Artificial Sequence
  Synthetic Polynucleotide
ugauauagacugaguucuggaacauga

SEQ ID NO: 716 - RNA, 27 nt, Artificial Sequence
  Synthetic Polynucleotide
gugauauagacugaguucuggaacaug

SEQ ID NO: 717 - RNA, 27 nt, Artificial Sequence
  Synthetic Polynucleotide
agugauauagacugaguucuggaacau

SEQ ID NO: 718 - RNA, 27 nt, Artificial Sequence
  Synthetic Polynucleotide
aagugauauagacugaguucuggaaca

SEQ ID NO: 719 - RNA, 27 nt, Artificial Sequence
  Synthetic Polynucleotide
gaaagugauauagacugaguucuggaa

SEQ ID NO: 720 - RNA, 27 nt, Artificial Sequence
  Synthetic Polynucleotide
aaggaaagugauauagacugaguucug

SEQ ID NO: 721 - RNA, 27 nt, Artificial Sequence
  Synthetic Polynucleotide
uucuuuccaaaggagaaaauguuaucc

SEQ ID NO: 722 - RNA, 27 nt, Artificial Sequence
  Synthetic Polynucleotide
cuucuuuccaaaggagaaaauguuauc

SEQ ID NO: 723 - RNA, 27 nt, Artificial Sequence
  Synthetic Polynucleotide
gcuucuuuccaaaggagaaaauguuau

SEQ ID NO: 724 - RNA, 27 nt, Artificial Sequence
  Synthetic Polynucleotide
cuuuuucaucaaucagcuucuuuccaa

SEQ ID NO: 725 - RNA, 27 nt, Artificial Sequence
  Synthetic Polynucleotide
acuuuuucaucaaucagcuucuuucca

SEQ ID NO: 726 - RNA, 27 nt, Artificial Sequence
  Synthetic Polynucleotide
uggacuuuuucaucaaucagcuucuuu

SEQ ID NO: 727 - RNA, 27 nt, Artificial Sequence
  Synthetic Polynucleotide
uacugagcaauucauuggucagcagga

SEQ ID NO: 728 - RNA, 27 nt, Artificial Sequence
  Synthetic Polynucleotide
aguacugagcaauucauuggucagcag

SEQ ID NO: 729 - RNA, 27 nt, Artificial Sequence
  Synthetic Polynucleotide
gaguacugagcaauucauuggucagca

SEQ ID NO: 730 - RNA, 27 nt, Artificial Sequence
  Synthetic Polynucleotide
cugaguacugagcaauucauuggucag

SEQ ID NO: 731 - RNA, 27 nt, Artificial Sequence
  Synthetic Polynucleotide
ccugaguacugagcaauucauugguca

SEQ ID NO: 732 - RNA, 27 nt, Artificial Sequence
  Synthetic Polynucleotide
uccugaguacugagcaauucauugguc

SEQ ID NO: 733 - RNA, 27 nt, Artificial Sequence
  Synthetic Polynucleotide
ucuccugaguacugagcaauucauugg

SEQ ID NO: 734 - RNA, 27 nt, Artificial Sequence
  Synthetic Polynucleotide
gucuccugaguacugagcaauucauug

SEQ ID NO: 735 - RNA, 27 nt, Artificial Sequence
  Synthetic Polynucleotide
ucaguuuccuuuucugugaugauccgg

SEQ ID NO: 736 - RNA, 27 nt, Artificial Sequence
  Synthetic Polynucleotide
uucaguuuccuuuucugugaugauccg

SEQ ID NO: 737 - RNA, 27 nt, Artificial Sequence
  Synthetic Polynucleotide
uaauuucaguuuccuuuucugugauga

SEQ ID NO: 738 - RNA, 27 nt, Artificial Sequence
  Synthetic Polynucleotide
uuaauuucaguuuccuuuucugugaug

SEQ ID NO: 739 - RNA, 27 nt, Artificial Sequence
  Synthetic Polynucleotide
auuaauuucaguuuccuuuucugugau

SEQ ID NO: 740 - RNA, 27 nt, Artificial Sequence
  Synthetic Polynucleotide
cauuaauuucaguuuccuuuucuguga

SEQ ID NO: 741 - RNA, 27 nt, Artificial Sequence
  Synthetic Polynucleotide
agccauuaauuucaguuuccuuuucug

SEQ ID NO: 742 - RNA, 27 nt, Artificial Sequence
  Synthetic Polynucleotide
aagccauuaauuucaguuuccuuuucu

SEQ ID NO: 743 - RNA, 27 nt, Artificial Sequence
  Synthetic Polynucleotide
gagaaagccauuaauuucaguuuccuu

SEQ ID NO: 744 - RNA, 27 nt, Artificial Sequence
  Synthetic Polynucleotide
guuauuauaaggugcucugcugucuua

SEQ ID NO: 745 - RNA, 27 nt, Artificial Sequence
  Synthetic Polynucleotide
uguuauuauaaggugcucugcugucuu

SEQ ID NO: 746 - RNA, 27 nt, Artificial Sequence
  Synthetic Polynucleotide
acuguuauuauaaggugcucugcuguc

SEQ ID NO: 747 - RNA, 27 nt, Artificial Sequence
  Synthetic Polynucleotide
gacuguuauuauaaggugcucugcugu

SEQ ID NO: 748 - RNA, 27 nt, Artificial Sequence
  Synthetic Polynucleotide
caaggacuguuauuauaaggugcucug

SEQ ID NO: 749 - RNA, 27 nt, Artificial Sequence
  Synthetic Polynucleotide
aaucauacccaaggacuguuauuauaa

SEQ ID NO: 750 - RNA, 27 nt, Artificial Sequence
  Synthetic Polynucleotide
auuuuaaaucauacccaaggacuguua

SEQ ID NO: 751 - RNA, 27 nt, Artificial Sequence
  Synthetic Polynucleotide
uauuuuaaaucauacccaaggacuguu

SEQ ID NO: 752 - RNA, 27 nt, Artificial Sequence
  Synthetic Polynucleotide
uuauuuuaaaucauacccaaggacugu

SEQ ID NO: 753 - RNA, 27 nt, Artificial Sequence
  Synthetic Polynucleotide
uuuauuuuaaaucauacccaaggacug

SEQ ID NO: 754 - RNA, 27 nt, Artificial Sequence
  Synthetic Polynucleotide
uuuuauuuuaaaucauacccaaggacu

SEQ ID NO: 755 - RNA, 27 nt, Artificial Sequence
  Synthetic Polynucleotide
aauuuuauuuuaaaucauacccaagga

SEQ ID NO: 756 - RNA, 27 nt, Artificial Sequence
  Synthetic Polynucleotide
aaauuuuauuuuaaaucauacccaagg

SEQ ID NO: 757 - RNA, 27 nt, Artificial Sequence
  Synthetic Polynucleotide
uaaauuuuauuuuaaaucauacccaag

SEQ ID NO: 758 - RNA, 27 nt, Artificial Sequence
  Synthetic Polynucleotide
uuaaauuuuauuuuaaaucauacccaa

SEQ ID NO: 759 - RNA, 36 nt, Artificial Sequence
  Synthetic Polynucleotide
cggaacgcuacaauuuuuaugcagccgaaaggcugc

SEQ ID NO: 760 - RNA, 36 nt, Artificial Sequence
  Synthetic Polynucleotide
acaauuuuuauuccagcuaugcagccgaaaggcugc

SEQ ID NO: 761 - RNA, 36 nt, Artificial Sequence
  Synthetic Polynucleotide
acgagguuaucagugacuuugcagccgaaaggcugc

SEQ ID NO: 762 - RNA, 36 nt, Artificial Sequence
  Synthetic Polynucleotide
agauccaggaggccuugcacgcagccgaaaggcugc

SEQ ID NO: 763 - RNA, 22 nt, Artificial Sequence
  Synthetic Polynucleotide
auaaaaauuguagcguuccggu

SEQ ID NO: 764 - RNA, 22 nt, Artificial Sequence
  Synthetic Polynucleotide
auagcuggaauaaaaauuguag

SEQ ID NO: 765 - RNA, 22 nt, Artificial Sequence
  Synthetic Polynucleotide
aaagucacugauaaccucguuu

SEQ ID NO: 766 - RNA, 22 nt, Artificial Sequence
  Synthetic Polynucleotide
gugcaaggccuccuggaucuca

SEQ ID NO: 767 - RNA, 37 nt, Homo sapiens
cugcaccaguuacaggugcuuuacaaggccaaguacg

SEQ ID NO: 768 - RNA, 31 nt, Homo sapiens
aaguacgguccaauguggauguccuacuuag

SEQ ID NO: 769 - RNA, 39 nt, Homo sapiens
ggcaaguacccaguacggaacgacauggagcuauggaag

SEQ ID NO: 770 - RNA, 64 nt, Homo sapiens
cagcgcucuauacggaugcuuucaaugaggugauugaugacuuuaugacucgacuggaccagcu

SEQ ID NO: 771 - RNA, 48 nt, Homo sapiens
ucggacauggcucaacucuucuacuacuuugccuuggaagcuauuugc

SEQ ID NO: 772 - RNA, 42 nt, Homo sapiens
gccuuggaagcuauuugcuacauccuguucgagaaacgcauu

SEQ ID NO: 773 - RNA, 47 nt, Homo sapiens
cagauccaucggguuaauguuccagaacucacucuaugccaccuucc

SEQ ID NO: 774 - RNA, 45 nt, Homo sapiens
ccuuugggaagaagcugauugaugagaagcucgaagauauggagg

SEQ ID NO: 775 - RNA, 29 nt, Homo sapiens
cugacaugggcccuguaccaccucucaaa

SEQ ID NO: 776 - RNA, 48 nt, Homo sapiens
aggacuuugcccacaugccguugcaaagcugugcuuaaggagacucug

SEQ ID NO: 777 - RNA, 53 nt, Homo sapiens
cccacaaacucccggaucauagaaaaggaaauugaaguugauggcuuccucuu

SEQ ID NO: 778 - RNA, 28 nt, Homo sapiens
gcaaggcugauccagaaguacaaggugg

SEQ ID NO: 779 - RNA, 31 nt, Homo sapiens
cgcauuguccugguucccaauaagaaagugg

SEQ ID NO: 780 - RNA, 46 nt, Homo sapiens
uuugccacuucuaucauuuuugagcaacucccucucagcuaaaagg

SEQ ID NO: 781 - RNA, 37 nt, Homo sapiens
cgcauugcuguccuuggguagaauauaaaauaaaggg

SEQ ID NO: 782 - RNA, 2388 nt, Homo sapiens
gggagaagccgagggcagcuuagccacggccgguucccguucccuccaggacgcgagggucgccuuggguggggaaccgcgaccgggcgaggaccuaucccgguguggggcuucccgauuucgaaagaaucucgcugcacccccgcccagaguucagaccaagcgaaaaguuauuugagaggccucgggggcgcggggugaggagucguggcggaggccuuggucggggcgccguggauauccccgagucaccgcgucccucuccugcagcucccgcgucgcugggaggagcgagggagcgagcgggaaggggucuagcuggccuuugcucggcccuccccagcgcccggcuuugaacccgcccugcacugcugucugggcggguccggggacucagcacucgacccaaaggugcaggcgcgcgagcacaacccauggcugcgcugggcugcgcgaggcugaggugggcgcugcgaggggccggccguggccucugcccccacggggccagagccaaggccgcgaucccugccgcccuccccucggacaaggccaccggagcucccggagccgggccugguguccggcggcggcaacggagcuuagaggagauuccacgucuaggacagcugcgcuucuucuuucagcuguucguucaaggcuaugcccugcaacugcaccaguuacaggugcuuuacaaggccaaguacgguccaauguggauguccuacuuagggccucagaugcacgugaaccuggccagugccccgcucuuggagcaagugaugcggcaagagggcaaguacccaguacggaacgacauggagcuauggaaggagcaccgggaccagcacgaccugaccuaugggccguucaccacggaaggacaccacugguaccagcugcgccaggcucugaaccagcgguugcugaagccagcggaagcagcgcucuauacggaugcuuucaaugaggugauugaugacuuuaugacucgacuggaccagcugcgggcagagagugcuucggggaaccaggugucggacauggcucaacucuucuacuacuuugccuuggaagcuauuugcuacauccuguucgagaaacgcauuggcugccugcagcgauccauccccgaggacaccgugaccuucgucagauccaucggguuaauguuccagaacucacucuaugccaccuuccuccccaaguggacucgccccgugcugccuuucuggaagcgauaccuggaugguuggaaugccaucuuuuccuuugggaagaagcugauugaugagaagcucgaagauauggaggcccaacugcaggcagcagggccagauggcauccaggugucuggcuaccugcacuucuuacuggccaguggacagcucaguccucgggaggccaugggcagccugccugagcugcucauggcuggaguggacacgacauccaacacgcugacaugggcccuguaccaccucucaaaggacccugagauccaggaggccuugcacgaggaagugguggguguggugccagccgggcaagugccccagcacaaggacuuugcccacaugccguugcucaaagcugugcuuaaggagacucugcgucucuacccugugguccccacaaacucccggaucauagaaaaggaaauugaaguugauggcuuccucuuccccaagaacacccaguuuguguucugccacuauguggugucccgggaccccacugccuucucugagccugaaagcuuccagccccaccgcuggcugagaaacagccagccugcuacccccaggauccagcacccauuuggcucugugcccuuuggcuaugggguccgggccugccugggccgcaggauugcagagcuggagaugcagcuacuccucgcaaggcugauccagaaguacaaggugguccuggccccggagacgggggaguugaagaguguggcccgcauuguccugguucccaauaagaaagugggccugcaguuccugcagagacagugcugagcugagucuccgccuugcuggggcuuguccuagaggcuccagcucuggcacagugguuccuggcugcugccaugucucagaugaggagggagagaaggaggccgccagacucgagaggugggaggaacuccuugcacacacccugagcuuuugccacuucuaucauuuuugagcaacucccucucagcuaaaaggccaccccuuuaucgcauugcuguccuuggguagaauauaaaauaaagggacuuuuauuucuuauuggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 783 - RNA, 2301 nt, Macaca mulatta
gggagaagccgggggcagcuuagucgcggccgguuccuguucccuucaggacgcgagggucgccuuggguggggaacccgcggccgggcgaggaccugucccaguguaggacuucccgguuucgaaagaaucucgcugcacccccgcccagaguucagaccaagcgaaaaguuauuugagaggccucgggggcgcggggugaggagucgguggcggaggccuuggucggggcgcgguggguaucccugagucaccgcgucccucuccugcagaccccacgacgcugggaggagcgagggagugagcgggaaggggucuggcuggccuuugcucggcccuccccagcgcccggccuugaacccgcccugagcugcugccugggcggguccggggguuccgcacucgacccaaaggugcaggcgggcgagcacaacccauggcugcgcugggcugcgcgagguugaggugggugcugcgaggggccggccguggccucugcccccacggggccagagccaaggccacgaucccuaccgcccuccccucggacaaggccaccgaggcucccggagccgggccugguauccggcggcggcaacggagcuuaaaggagauuccacgucuagggcagcugcgcuucuucuuucagcuguuuguucaaggcuaugcccugcagcugcaccaguuacaggugcuuuacaaggccaaguacgguccaauguggauguccuacuuagggccucagaugcacgugaaccuggccagugccccgcucuuggagcaagugaugcggcaagagggcaaguacccaguacggaacgacauggagcuauggaaggagcaccgggaccugcacgaccugaccuaugggccguucaccacggaaggacaccacugguaccagcugcgccaggcucugaaccagcgguugcugaagccagcggaggcagcgcucuauacggaugcuuucaaugaggugauugaugauuuuaugauucgacuggaccagcugcgggcagagagugcuucggggaaccaggugucggacacggcucaacucuucuacuacuuugccuuggaagcuauuugcuacauccuguucgagaaacgcauuggcugccugcagcgauccauccccgaggacaccgugaccuuugucagauccaucggguuaauguuccagaacucacucuaugccaccuuccuccccaaguggacccgccccgugcugccuuucuggaagcgauaccuggaugguuggaaugccaucuuuuccuucgggaagaagcugauugaugagaaacucgaggauauggaggcccaacugcaggcagaggggccagaugguguccaggugucuggcuaccugcacuucuugcuggccaguggacagcucaguccucgggaggccaugggcagccugccugaacugcucauggcuggaguggacacgacauccaacacgcugacaugggcccuguaccaccucucaaaggacccugagauccaggaggccuugcacgaggaagugguggguguggugccagccgggcaggugccccagcacaaagacuuugcccacuugccgcugcucaaagcugugcuuaaggagacucugcgccucuacccugugguccccacaaauucccggaucauagaaaaggaaauugaaguugauggcuuccucuuccccaagaauacccaguuuguguucugccacuacguggugucccgggaccccaccaccuucucugaaccugagagcuuccagccccaccgcuggcugaggagcagccagccugcuacccccaggauccaacacccauuuggcucugugcccuuuggcuaugggguccgggccugccugggccgcaggauugcagagcuggagaugcagcuacuccuggcaaggcugauccagaaguacaaggugguccuggccccagagacgggggaguugaagaguguggcccgcauuguccugguucccaauaagaaagugggccugcaguuccugcagagacagugcugagcugaguccccaccuugcuggggcuuguccuagaggcuccagcccuggcauagugguuccuggcugccgccugucucagaugaggagggagagaaggaggccgccagacuugagaggugggaggaacucccugcacuucccacagacccugagcuuuugccacuucuaucauuuuugagcaacucccucucagauaaaaggccaccgcuuuaucgcauugcuguccuuggguagaauauaaaauaaagggacuuuuauuucuuaucgga

SEQ ID NO: 784 - RNA, 1936 nt, Mus musculus
gucgccaggucgcaaagcccgaguucugucugccuccuugccuggauagggcucauagucucuggaucuaaacucuuggcuucucgggcacgauccauggcugcguggagccgcacgaggcugagauggacgcuccuggacccgcguguggugggccguggccucugcccacaaggggccagagccaaggccacgaucccugcagcccuccaggcacaggagaguacggaggguccaggaacaggucaagaccggccgcgccugcggaguccggcggagcuuccggggaccggaacgcuacaauuuuuauuccagcuauuucuacaaggcuaugugcugcacuugcccgaccuccaggugcugaacaagaccaaguaugguccaauguggacaaccuccuuugggacuuacaccaaugugaaucuggcuagugccccacucuuggagcaagugaugagacaggagggcaaguacccaauaagagaccacauggaucaguggaaggaccaccgagaccacaagggccucaccuaugggaucuucaucgcacaaggagagcaaugguaccaucugcgucaggcuuugaaacagaggcugcugaagccugacgaggccgcgcucuacacggaugccuuaaacgagguuaucagugacuuuaucacccggcuggaccaggugcgggcagagagugaaucaggggaccaggugccagacauggcucaucuucucuaccaccuugccuuggaagccaucaccuauauccuguuugagaaaaggauuggcugccugaaacccuccauuccugaggacacugccgccuucaucagaucuguugcaaucauguuccagaacucagucuauaucacuuuccuucccaaauggacgcguccucugcugcccuuuuggaagcgauaccugaauggcugggauaacauuuucuccuuuggaaagaagcugauugaugaaaaaguccaggagcuaaaagcccagcuacaggaaacugggccagauggaguccggguaucuggcuaccugcacuuccugcugaccaaugaauugcucaguacucaggagaccaucggcaccuuuccugagcugcuuuuggcugggguggacacgacauccaacacacugacaugggcccuguaccaccuuucaaagagcccagagauccaggaggccuugcacaaggaagugacugguguggugcccuucgggaaggugccccagcacaaggacuuugcccacaugccucugcuaaaagcugugauuaaggagacccugcgccucuacccugugguucccacaaacucccggaucaucacagaaaaggaaacugaaauuaauggcuuucucuuccccaagaauacacaguuuguguuaugccacuacguggugucccgggaucccagugucuuuccugagcccaacagcuuccagccucaccgauggcugaggaagaaagaggcugauaacccugggauccuacauccauucggcucugugcccuucggcuaugggguucggucuugccugggucggaggauugcagaacuggagaugcaacugaugcugucaaggcugguacagaaguaugagauugcccuggcucccgggaugggagaaguaaagacugugucccgcaucguccugguucccagcaagaaggugaggcugcauuuucugcagagacaguaguaccgagcugggcucccgccuucauggagcucguccagaagcccuggcacagaaguucuuggccagucucaugucacaugucacgaugucagauucaacaggagaacucugugcccuuccuauagacaccaaaugucuggcacaaucucuacugagcagcacccacuuaagacagcagagcaccuuauaauaacaguccuuggguaugauuuaaaauaaaauuuaaaauucaaguuuu

SEQ ID NO: 785 - RNA, 35 nt, Artificial Sequence
  Synthetic Polynucleotide
ugcuacauccuguucgagagcagccgaaaggcugc

SEQ ID NO: 786 - RNA, 21 nt, Artificial Sequence
  Synthetic Polynucleotide
ucucgaacaggauguagcaaa

SEQ ID NO: 787 - RNA, 35 nt, Artificial Sequence
  Synthetic Polynucleotide
cagaacucacucuaugccagcagccgaaaggcugc

SEQ ID NO: 788 - RNA, 21 nt, Artificial Sequence
  Synthetic Polynucleotide
uggcauagagugaguucugga

SEQ ID NO: 789 - RNA, 25 nt, Artificial Sequence
  Synthetic Polynucleotide
cggaacgcuacaauuuuuauuccag

SEQ ID NO: 790 - RNA, 27 nt, Artificial Sequence
  Synthetic Polynucleotide
cuggaauaaaaauuguagcguuccggu

SEQ ID NO: 791 - RNA, 22 nt, Artificial Sequence
  Synthetic Polynucleotide
uucucgaacaggauguagcagg

SEQ ID NO: 792 - RNA, 22 nt, Artificial Sequence
  Synthetic Polynucleotide
auagcuggaauaaaaauugugg